(19) United States
(12) Patent Application Publication
(10) Pub. No.: US 2026/0335465 A1
(43) Pub. Date: Oct. 29, 2026

(54) CELL TYPE SPECIFIC ENHANCERS, METHODS AND USES RELATED THERETO

(71) Applicants: The Broad Institute, Inc., Cambridge, MA (US); President and Fellows of Harvard College, Cambridge, MA (US)

(72) Inventors: Gordon Fishell, Cambridge, MA (US); Yating Wang, Cambridge, MA (US); Min Dai, Cambridge, MA (US)

(73) Assignees: The Broad Institute, Inc., Cambridge, MA (US); President and Fellows of Harvard College, Cambridge, MA (US)

(21) Appl. No.: 19/696,009

(22) Filed: Jun. 2, 2026

Related U.S. Application Data
(63) Continuation of application No. PCT/US2024/058378, filed on Dec. 4, 2024.
(60) Provisional application No. 63/608,039, filed on Dec. 8, 2023.

(51) Int. Cl. A61K 48/00; A61P 25/00; C12N 15/86; G16B 30/00

(52) U.S. Cl. CPC A61K 48/0058; A61K 48/0033; A61P 25/00; C12N 15/86; G16B 30/00; C12N 2750/14143; C12N 2830/008

(57) ABSTRACT

Provided are isolated, cloned enhancer element sequences that target certain neuronal cell types, subtypes, and/or populations in the brain and CNS. The isolated and cloned enhancer elements can target a gene of interest, such as a therapeutic gene or an effector gene, to be expressed in the certain cell types. Virus vectors containing the isolated, cloned enhancer element sequences, particularly, recombinant adeno-associated virus (rAAV) vectors, and a gene or polynucleotide to be expressed in a given neuronal cell type are described. The rAAV vectors, compositions and methods are useful for treating subjects afflicted with neuropsychiatric and neuropathological diseases, disorders and conditions and symptoms thereof.

### CROSS REFERENCE TO RELATED APPLICATION

This application is a continuation under 35 U.S.C. § 111 (a) of PCT International Patent Application No. PCT/US2024/058378, filed Dec. 4, 2024, designating the United States and published in English, which claims priority to and the benefit of U.S. Provisional Application No. 63/608,039, filed Dec. 8, 2023, the entire contents of each of which are incorporated by reference herein.

### STATEMENT REGARDING FEDERALLY SPONSORED RESEARCH

The invention was made with government support under grant numbers UF1MH130701 and UH3MH120096 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Dec. 17, 2024, is named 167741-052201PCT_SL.xml and is 27,062 bytes in size.

### BACKGROUND

Recombinant AAV (rAAV) and rAAV vectors can drive long-term gene expression in cells and hence has become a popular gene delivery vector for research and gene therapy. However, there is a need to regulate transgenes carried in rAAV vectors for expression in a tissue/cell-type specific or context-dependent manner to better leverage rAAV for research and therapeutic use. While efforts have been made to modify the AAV capsid, the extent of specificity offered by capsid choice is relatively limited.

Enhancer sequences are non-coding elements that can regulate gene expression and can control the activity or spatial-temporal pattern of gene expression. Viral tools such as rAAV to access and manipulate fine neuronal subtypes are still limited. Such viral tools are needed for the continued study of cortical interneurons and the neural circuits to which they contribute, and particularly, for tissue/cell-type specific or context dependent therapeutic use. Also needed in the art are enhancer sequences that are highly selective for distinct cortical interneuronal cell types and populations that are effective across species. The products, compositions, methods described herein address and meet these needs.

### SUMMARY

Featured herein are isolated enhancer element sequences that target certain neuronal cells, cell types, subtypes, or populations, such as neuronal and interneuronal cell types and/or cell populations of the brain and/or central nervous system (CNS), and function to restrict and regulate the expression of genes (polynucleotides encoding gene products) in these cells. In embodiments, the enhancer element sequences are isolated, cloned (e.g., molecularly cloned) and can be components of viral vectors, e.g., recombinant adeno-associated virus (rAAV) vectors, virus particles, and compositions and methods of use. The rAAV vectors contain (are molecularly engineered to contain) at least one gene of interest (a transgene, such as, for example, a therapeutic gene, an effector gene, a reporter gene, or a gene encoding a regulatory protein or a protein, polypeptide, or peptide). Such a gene of interest is expressed in the cells and/or has an activity or function in the cells, e.g., a therapeutic, corrective, effector, or treatment activity or function in a cell. In certain embodiments, without limitation, the gene or transgene comprises a polynucleotide encoding the hM3Dq modified muscarinic receptor (Gq-DREADD), a polynucleotide encoding the pharmacologically selective actuator molecule (PSAM), a polynucleotide encoding a clustered regularly interspaced short palindromic repeats-Cas9 (CRISPR-Cas9) protein or variant thereof, a polynucleotide encoding a Zinc Finger Protein, a polynucleotide encoding a Transcription activator-like effector nuclease (TALEN), or engineered forms thereof, for gene editing, base editing, or gene expression modulation for functional activity in a targeted cell (neuronal cell type), or a functional portion thereof. An isolated and cloned enhancer element sequence as described herein targets, directs, regulates, modulates, or restricts expression of the gene of interest to a particular cell type or population, e.g., an intended target neuronal cell or cell population. In an embodiment, a gene of interest (transgene) may encode an effector nuclease, e.g., Cas9 and the like, to disrupt a harmful or mutated gene, or to repair a mutated gene or polynucleotide, e.g., a gene or polynucleotide encoding a receptor protein, a channel protein, such as, for example, an ion channel or a ligand-gated ion channel, a transcription factor protein, or a disease-causing protein, and the like. It will be understood that the terms “enhancer,” “enhancer element,” “enhancer sequence,” enhancer element sequence (or polynucleotide),” and the like, are used interchangeably herein. Also as used herein, the term “cell” may refer to a cell type or subtype.

In an aspect, an isolated, cloned enhancer element comprising a polynucleotide sequence having at least 75% identity to a polynucleotide sequence set forth in any one of SEQ ID NOs: 1-16, wherein the enhancer element targets expression of a gene or polynucleotide of interest in a neuronal or interneuronal cell type, subtype, or population. In an embodiment, the enhancer element comprises a polynucleotide sequence having at least 85% identity to a polynucleotide sequence set forth in any one of SEQ ID NOs: 1-16. In an embodiment, the enhancer element comprises a polynucleotide sequence having at least 90% identity to a polynucleotide sequence set forth in any one of SEQ ID NOs: 1-16. In an embodiment, the enhancer element comprises a polynucleotide sequence having at least 95% identity to a polynucleotide sequence set forth in any one of SEQ ID NOs: 1-16. In an embodiment, the enhancer element comprises a polynucleotide sequence having at least 98% identity to a polynucleotide sequence set forth in any one of SEQ ID NOs: 1-16. In an embodiment, the enhancer element comprises or consists of a polynucleotide sequence set forth in any one of SEQ ID NOs: 1-16. In an embodiment, the neuronal cell type or population comprises inhibitory GABA-ergic neurons or interneurons. In an embodiment, the inhibitory GABA-ergic neurons are selected from parvalbumin (PV)-expressing basket interneurons, parvalbumin (PV)-expressing chandelier interneurons, Somatostatin (SST)-expressing interneurons, Vasoactive Intestinal Peptide (VIP)-expressing interneurons, or Lamp5-expressing interneurons. In embodiments, the enhancer element comprises SEQ ID NO: 1 or 9 having specificity for parvalbumin (PV)-expressing basket interneurons; SEQ ID NO: 2 or 10 having specificity for parvalbumin (PV)-expressing chandelier interneurons; SEQ ID NO: 3 or 11 having specificity for somatostatin (SST)-expressing interneurons; SEQ ID NO: 8 or 16 having specificity for vasoactive intestinal peptide (VIP)-expressing interneurons; and any one of SEQ ID NOs: 4-7 or 12-15 having specificity for Lamp5-expressing interneurons.

In an embodiment of the above-delineated aspect and/or embodiments thereof, the enhancer element regulates or restricts expression of the gene of interest in the neuronal or interneuronal cell type, subtype, or population. In an embodiment, the gene or polynucleotide of interest is a transgene, a reporter gene, or effector gene or polynucleotide. In embodiments, the gene or polynucleotide of interest is selected from a therapeutic gene, a reporter gene, an effector gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof.

Provided in another aspect is an isolated, cloned enhancer element comprising a polynucleotide sequence of SEQ ID NO: 1 or 9 having specificity for parvalbumin (PV)-expressing basket interneurons in the brain.

Provided in another aspect is an isolated, cloned enhancer element comprising a polynucleotide sequence of SEQ ID NO: 2 or 10 having specificity for parvalbumin (PV)-expressing chandelier interneurons in the brain.

Provided in another aspect is an isolated, cloned enhancer element comprising a polynucleotide sequence of SEQ ID NO: 3 or 11 having specificity for somatostatin (SST)-expressing interneurons in the brain.

Provided in another aspect is an isolated, cloned enhancer element comprising a polynucleotide sequence of SEQ ID NO: 8 or 16 having specificity for vasoactive intestinal peptide (VIP)-expressing interneurons in the brain.

Provided in another aspect is an isolated, cloned enhancer element comprising a polynucleotide sequence of any one of SEQ ID NOs: 4-7 or 12-15 having specificity for Lamp5-expressing interneurons in the brain.

In another aspect, a viral vector comprising the isolated enhancer element of any one of the above-delineated aspects and/or embodiments thereof is provided. In an embodiment, the viral vector further comprises a gene or polynucleotide (e.g., a transgene) of interest. In another aspect, a viral vector comprising an enhancer element comprising the sequence of any one of SEQ ID NOs: 1-16 is provided. In an embodiment, the viral vector further comprises a gene or polynucleotide (e.g., a transgene) of interest.

In an embodiment of any of the above-delineated aspects of the viral vector and/or embodiments thereof, the enhancer element comprises SEQ ID NO: 2 or SEQ ID NO: 10 and has at least 64% specificity for parvalbumin (PV)-expressing chandelier interneurons in the brain cortex. In an embodiment of any of the above-delineated aspects of the viral vector and/or embodiments thereof, the enhancer element comprises SEQ ID NO: 8 or SEQ ID NO: 16 and has at least 60% specificity for vasoactive intestinal peptide (VIP)-expressing interneurons in the brain cortex. In an embodiment of any of the above-delineated aspects of the viral vector and/or embodiments thereof, the enhancer element comprises any one of SEQ ID NOs: 4-7 or SEQ ID NOs: 12-15 and has at least 60%-80% specificity for Lamp5-expressing interneurons in the brain cortex.

In an embodiment of any of the above-delineated aspects of the viral vector and/or embodiments thereof, the viral vector is a lentivirus vector, an adeno-associated virus (AAV) vector, or a recombinant adeno-associated virus (rAAV) vector. In an embodiment, the viral vector is a recombinant adeno-associated virus (rAAV) vector. In an embodiment, the capacity of the viral vector to package polynucleotide sequences of greater than about 4.7 kb comprises reassembly of multiple rAAV vectors by homologous recombination or by splicing mediated by acceptor sites. In an embodiment, the vector delivers the gene of interest to GABA-ergic neurons or interneurons in the brain, and wherein the gene or polynucleotide of interest is functionally expressed in the neurons or interneurons following administration of the vector to a subject. In an embodiment, the subject is a mammalian subject or a human patient.

In another aspect, a viral vector comprising an isolated, cloned enhancer polynucleotide sequence selected from any one of SEQ ID NOs: 1-16, or a functional portion thereof, and a gene or polynucleotide of interest for expression in a GABA-ergic interneuron cell, cell type or subtype of the brain cortex is provided.

In another aspect, a viral vector comprising an isolated enhancer polynucleotide sequence of SEQ ID NOs: 1 or 9, or a functional portion thereof, and a gene or polynucleotide of interest to be expressed in parvalbumin (PV)-expressing basket cells of the brain cortex is provided.

In another aspect, a viral vector comprising an isolated enhancer polynucleotide sequence of SEQ ID NOs: 2 or 10, or a functional portion thereof, and a gene or polynucleotide of interest to be expressed in parvalbumin (PV)-expressing chandelier cells of the brain cortex is provided.

In another aspect, a viral vector comprising an isolated enhancer polynucleotide sequence of SEQ ID NO: 3 or 11, or a functional portion thereof, and a gene or polynucleotide of interest to be expressed in somatostatin (SST)-expressing cells of the brain cortex is provided.

In another aspect, a viral vector comprising an isolated enhancer polynucleotide sequence of any one of SEQ ID NOs: 4-7 or SEQ ID NOs: 12-15, or a functional portion thereof, and a gene or polynucleotide of interest to be expressed in Lamp5-expressing basket cells of the brain cortex is provided.

In another aspect, a viral vector comprising an isolated enhancer polynucleotide sequence of SEQ ID NO: 8 or 16, or a functional portion thereof, and a gene or polynucleotide of interest to be expressed in vasoactive intestinal peptide (VIP)-expressing cells of the brain cortex is provided.

In an embodiment of the above-delineated aspects and/or embodiments thereof, the viral vector is an adeno-associated viral vector (AAV) or a recombinant adeno-associated viral vector (rAAV), or a virus particle or virus-like particle thereof. In an embodiment of the above-delineated aspects of the viral vector and/or embodiments thereof, the gene or polynucleotide of interest is selected from a reporter gene, a therapeutic gene encoding a therapeutically or enzymatically active polypeptide, an effector gene or polynucleotide, a polynucleotide encoding a CRISPR-Cas9 protein, a polynucleotide encoding a Zinc Finger Protein, a polynucleotide encoding a Transcription activator-like effector nuclease (TALEN), or an engineered form thereof.

In another aspect a viral particle or virus-like particle comprising the viral vector of any one of the above-delineated aspects and/or embodiments thereof is provided.

In another aspect, a cell comprising the viral vector of any one of the above-delineated aspects and/or embodiments thereof is provided.

In another aspect, a cell comprising the viral particle or virus-like particle of the above-delineated aspect and/or embodiments thereof is provided.

In another aspect a pharmaceutical composition comprising the viral vector of any one of the above-delineated aspects and/or embodiments thereof, and a pharmaceutically acceptable vehicle, carrier, or diluent is provided. In an embodiment, the pharmaceutical composition is in liquid dosage form.

In another aspect, a pharmaceutical composition comprising the viral particle or virus-like particle of the above-delineated aspect and/or embodiments thereof, and a pharmaceutically acceptable vehicle, carrier, or diluent is provided. In an embodiment, the pharmaceutical composition is in liquid dosage form.

In another aspect, a pharmaceutical composition comprising the cell of any one of the above-delineated aspects and/or embodiments thereof, and a pharmaceutically acceptable vehicle, carrier, or diluent is provided.

In an aspect, a method of restoring normal levels of target gene expression in GABA-ergic neuronal cells in which expression levels of the gene are deficient or defective is provided, in which the method involves contacting the cells with an effective amount of the viral vector of any one of the above-delineated aspects and/or an embodiment thereof, a viral particle, a virus-like particle, or a pharmaceutical composition thereof, to restore normal or essentially normal levels of expression of the target gene in the GABA-ergic neuronal cells. In an embodiment of the method, the viral vector, viral particle, or virus-like particle comprises a recombinant adeno-associated virus (rAAV).

In an aspect, a method of delivering a gene or polynucleotide of interest for restricted expression in a GABA-ergic neuronal cell is provided, in which the method involves contacting the GABA-ergic neuronal cell with a recombinant adeno-associated virus (rAAV) vector, a viral particle, a virus-like particle, or a pharmaceutical composition thereof, comprising a polynucleotide sequence of the gene or polynucleotide of interest, or a functional portion thereof, and an enhancer element polynucleotide sequence selected from one or more of SEQ ID NOs: 1-16 that restricts expression of the gene or polynucleotide of interest in target GABA-ergic interneuron cells of the cerebral cortex of the subject. In accordance with the method, the gene or polynucleotide of interest (e.g., a transgene) is delivered to the GABA-ergic neuronal cell in the subject. In an embodiment of the method, the GABA-ergic neuronal cell is selected from a parvalbumin (PV)-expressing basket interneuron, a parvalbumin (PV)-expressing chandelier interneuron, a Somatostatin (SST)-expressing interneuron, a vasoactive intestinal peptide (VIP)-expressing interneuron, or a Lamp5-expressing interneuron. In an embodiment of the method, the recombinant adeno-associated virus (rAAV) vector, viral particle, virus-like particle, or a pharmaceutical composition thereof comprises the enhancer element of SEQ ID NO: 1 or 9 and a gene or polynucleotide of interest for delivery to PV-expressing basket interneurons. In an embodiment of the method, the recombinant adeno-associated virus (rAAV) vector, viral particle, virus-like particle, or a pharmaceutical composition thereof comprises the enhancer element of SEQ ID NO: 2 or 10 and a gene or polynucleotide of interest for delivery to PV-expressing chandelier interneurons. In an embodiment of the method, the recombinant adeno-associated virus (rAAV) vector, viral particle, virus-like particle, or a pharmaceutical composition thereof comprises the enhancer element of SEQ ID NO: 3 or 11 and a gene or polynucleotide of interest for delivery to SST-expressing interneurons. In an embodiment of the method, the recombinant adeno-associated virus (rAAV) vector, viral particle, virus-like particle, or a pharmaceutical composition thereof comprises the enhancer element of any one of SEQ ID NOs: 4-7 or 12-15 and a gene or polynucleotide of interest for delivery to Lamp5-expressing interneurons. In an embodiment of the method, the recombinant adeno-associated virus (rAAV) vector, viral particle, virus-like particle, or a pharmaceutical composition thereof comprises the enhancer element of SEQ ID NO: 8 or 16 and a gene or polynucleotide of interest for delivery to VIP-expressing interneurons.

In embodiments of the above-delineated methods and/or embodiments thereof, the viral vector, viral particle, virus-like particle, or a pharmaceutical composition thereof, is administered systemically, parenterally, intravenously, or intracerebrally. In an embodiment of the above-delineated methods and/or embodiments thereof, the viral vector, viral particle, virus-like particle, or a pharmaceutical composition thereof, is administered as a prophylactic or a therapeutic. In embodiments of the above-delineated methods and/or embodiments thereof, the gene or polynucleotide of interest is selected from a therapeutic gene, a reporter gene, an effector gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof. In embodiments, the gene or polynucleotide of interest comprises a therapeutic gene that encodes a therapeutically or enzymatically active, functional, and/or beneficial polypeptide.

In another aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of the viral vector, viral particle, or virus-like particle of any one of the above-delineated aspects and/or embodiments thereof, and a pharmaceutically acceptable carrier, excipient, or vehicle thereof.

In another aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of a delivery vehicle, viral vector, virus-like particle, or viral particle of any of the above-delineated aspects and embodiments thereof, or a pharmaceutical composition thereof, which contains the isolated enhancer element of any one the above-delineated aspects and/or embodiments thereof, and a gene of interest.

In an aspect, a method of treating, abating, or ameliorating a neurological, neurodevelopmental, neurodegenerative, neuropsychiatric, neurogenetic, neuromuscular, or movement disease, disorder, or pathology, and/or the symptoms thereof, in a subject is provided, in which the method involves administering to a subject in need thereof an effective amount of a delivery vehicle or vector, or a pharmaceutical composition thereof, comprising an isolated, cloned enhancer element comprising (i) SEQ ID NO: 1 or SEQ ID NO: 9 having specificity for PV-expressing basket interneurons; (ii) SEQ ID NO: 2 or SEQ ID NO: 10 having specificity for to PV-expressing chandelier interneurons; (iii) SEQ ID NO: 3 or 11 having specificity for SST-expressing interneurons; (iv) any one of SEQ ID NOs: 4-7 or 12-15 having specificity for Lamp5-expressing interneurons; or (v) SEQ ID NO: 8 or 16 having specificity for VIP-expressing interneurons; and a gene or polynucleotide of interest. In an embodiment of the method, the disease, disorder, or pathology is selected from one or more of ataxia, dystonia, epilepsy, seizures, Alzheimer's disease, tremors, Essential Tremor, Lewy Body dementia, motor stereotypies and Parkinson's Disease, obsessive-compulsive disorder (OCD) and/or the symptoms thereof. In an embodiment, the isolated enhancer element is cloned into the delivery vehicle or vector, e.g., a viral vector, such as an adeno-associated virus (AAV) vector or a recombinant AAV (rAAV) vector.

In embodiments of the above delineated methods and/or embodiments thereof, the disease, disorder, or pathology is selected from Alzheimer's disease, Parkinson's disease, dystonia, amyotrophic lateral sclerosis (ALS), bipolar disorder, Down Syndrome, epilepsy, seizures, a cognitive disorder, and/or a neuropsychiatric disorder. In other embodiments, the disease, disorder, or pathology is selected from ataxia, tremors, Essential Tremor, Lewy Body dementia, motor stereotypies, obsessive-compulsive disorder (OCD), and/or the symptoms thereof. In an embodiment of the methods, the symptoms of the disease, disorder, or pathology are reduced, abated, or alleviated in the subject. In an embodiment, the subject is a mammalian subject, a human subject or a human patient.

In an embodiment of any one of the above-delineated aspects describing an enhancer element, viral vector, methods, and/or embodiments thereof, the gene or polynucleotide of interest is a therapeutic gene, a reporter gene, an effector gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof. In an embodiment, the gene or polynucleotide of interest is a therapeutic gene that encodes a therapeutically or enzymatically active, functional, and/or beneficial polypeptide.

In an aspect, an isolated, cloned enhancer element comprising the polynucleotide sequence of SEQ ID NO: 3 or 11, or a viral vector comprising the enhancer element, or a functional portion thereof is provided, wherein the enhancer element targets somatostatin (SST)-expressing interneurons expressing one or more of Satb1, Hpse, Ccna1, Calb1, or Smc2. In an embodiment, the enhancer element targets SST interneuron cells expressing the target gene Satb1 (Special AT-Rich Sequence Binding Protein 1 (Binds To Nuclear Matrix/Scaffold-Associating DNA)). The Satb1 gene product is a matrix protein which binds nuclear matrix and scaffold-associating DNAs. Diseases associated with SATB1 include Developmental Delay With Dysmorphic Facies And Dental Anomalies (DEFDA), characterized by generally mild global developmental delay with variably impaired intellectual development, and Kohlschutter-Tonz Syndrome-Like (KTZSL). KTZSL is characterized by global developmental delay with moderately to severely impaired intellectual development, poor or absent speech, and delayed motor skills. Early-onset epilepsy is common and may be refractory to treatment, leading to epileptic encephalopathy. In an embodiment, the enhancer element targets SST interneuron cells expressing the target gene Hpse (Heparinase). The Hpse gene product is an endoglycosidase that cleaves heparan sulfate proteoglycans (HSPGs) into heparin sulfate side chains and core proteoglycans and participates in extracellular matrix (ECM) degradation and remodeling. Diseases associated with HPSE include Urofacial Syndrome 1 (ochoa syndrome, a rare condition that causes unusual facial expressions and problems with urination) and Gastric Signet Ring Cell Adenocarcinoma. In an embodiment, the enhancer element targets SST interneuron cells expressing the target gene Ccna1 (Cyclin A1). The Cenal gene product belongs to the highly conserved cyclin family, whose members are characterized by a periodicity in protein abundance through the cell cycle. The cyclin encoded by Ccna1 is expressed in testis and brain. Diseases associated with Cenal include myeloid leukemia and testicular cancer. In an embodiment, the enhancer element targets SST interneuron cells expressing the target gene Calb1 (Calbidin 1), a Vitamin D-dependent calcium-binding protein. The Calb1 gene product is a member of the calcium-binding protein superfamily that includes calmodulin and troponin C. The Calb1 product plays a role in buffering entry of calcium upon stimulation of glutamate receptors. Depletion of this protein was noted in patients with Huntington disease. Diseases associated with Calb1 include Cerebellar Disease (ataxias, dysarthria and cerebellar cognitive affective syndrome) and Temporal Lobe Epilepsy. In an embodiment, the enhancer element targets SST interneuron cells expressing the target gene Smc2 (Structural Maintenance of Chromosomes 2). The Smc2 gene product is a central component of the condensin complex, required for conversion of interphase chromatin into mitotic-like condense chromosomes. Diseases associated with SMC2 include Pleural Empyema (a rare pulmonary condition characterized by accumulation of pus in the pleural cavity, often as a consequence of pneumonia) and Progeroid Syndrome (rare genetic disorders that mimic premature aging).

In an aspect, an isolated, cloned enhancer element comprising the polynucleotide sequence of any one of SEQ ID NOs: 1, 2, 9, or 10, or a viral vector comprising the enhancer element, or a functional portion thereof, wherein the enhancer element targets parvalbumin (PV)-expressing interneurons expressing one or more of Prss23, Lpl, Cntnap5b, Plcxd3, or Elf5. In an embodiment, the enhancer element targets PV interneuron cells expressing the target gene Prss23 (serine protease 23, a member of the trypsin family of serine proteases). Examples of diseases associated with Prss23 include Coats Disease, an idiopathic disorder characterized by retinal telangiectasia with deposition of intraretinal or subretinal exudates, which can lead to retinal detachment and unilateral blindness; and exudative vitreoretinopathy, which is an inherited disorder characterized by the incomplete development of the retinal vasculature. In an embodiment, the enhancer element targets PV interneuron cells expressing the target gene Lpl (lipoprotein lipase). The Lpl gene product has the dual functions of triglyceride hydrolase and ligand/bridging factor for receptor-mediated lipoprotein uptake. Severe mutations that cause Lpl deficiency result in type I hyperlipoproteinemia, while less extreme mutations in Lpl are linked to many disorders of lipoprotein metabolism. In an embodiment, the enhancer element targets PV interneuron cells expressing the target gene Cntnap5b (Contactin associated protein family member 5). The Cntnap5b gene product belongs to the neurexin family, members of which function in the vertebrate nervous system as cell adhesion molecules and receptors. Diseases associated with CNTNAP5 include Posterior Cortical Atrophy, a rare neurologic disease characterized by impairment of higher visual processing skills and other posterior cortical functions without any evidence of ocular abnormalities, relatively intact memory and language in the early stages, and atrophy of posterior brain regions; and Dyslexia, a brain-based type of learning disability that specifically impairs a person's ability to read. Cntnap5b may also play a role in the correct development and proper functioning of the peripheral and central nervous system and be involved in cell adhesion and intercellular communication. In an embodiment, the enhancer element targets PV interneuron cells expressing the target gene Plexd3 (Phosphatidylinositol Specific Phospholipase C X Domain Containing 3). The Plcxd3 gene product is one of a group of enzymes that hydrolyze phospholipids into fatty acids and other lipophilic molecules. Diseases associated with PLCXD3 include Creutzfeldt-Jakob Disease (CJD), a rare, degenerative, fatal brain disorder. The three major categories of CJD include sporadic (the most common form, in which people do not have any known risk factors for the disease); hereditary (in which the person has a family member with the disease and tests positive for a genetic mutation associated with CJD), and acquired (in which the disease is transmitted by exposure to brain and nervous system tissue, usually through certain medical procedures). A variant CJD form can be acquired by eating meat from cattle affected by a disease similar to CJD, called bovine spongiform encephalopathy (commonly called “mad cow” disease). In an embodiment, the enhancer element targets PV interneuron cells expressing the target gene Elf5 (E74-like Factor 5, Ets domain transcription factor). The Elf5 gene product is a member of an epithelium-specific subclass of the Ets transcription factor family. Diseases associated with ELF5 include Down Syndrome and Isolated Growth Hormone Deficiency, Type Ii.

In an aspect, an isolated, cloned enhancer element comprising the polynucleotide sequence of SEQ ID NO: 8 or 16, or a viral vector comprising the enhancer element, or a functional portion thereof, wherein the enhancer element targets Vaso-active Intestinal Peptide (VIP) interneurons expressing one or more of Proxl, Vip, Npy5r, or Grpr. In an embodiment, the enhancer element targets VIP interneuron cells expressing the target gene Proxl ((Prospero Homeobox 1)). The Proxl gene product is a member of the homeobox transcription factor family and plays a critical role in embryonic development and functions as a key regulatory protein in neurogenesis and the development of the heart, eye lens, liver, pancreas and the lymphatic system. Involved in the regulation of the circadian rhythm. In an embodiment, the enhancer element targets VIP interneuron cells expressing the target gene Vip (vasoactive intestinal peptide). The Vip gene product is a member of the glucagon family. Diseases associated with VIP include Vipoma (a rare cancer caused by a type of pancreatic neuroendocrine tumor, which secretes VIP, a hormone that stimulates the secretion (and inhibits the absorption) of sodium, chloride, potassium and water within the small intestine) and Pancreatic Cholera, a disease related to related to secretory diarrhea and diarrhea. VIP causes vasodilation, lowers arterial blood pressure, stimulates myocardial contractility, increases glycogenolysis and relaxes the smooth muscle of trachea, stomach and gall bladder. In an embodiment, the enhancer element targets VIP interneuron cells expressing the target gene Npy5r (Neuropeptide Y Receptor Type 5). The Npybr gene product is a receptor for neuropeptide Y and peptide YY. The encoded protein appears to be involved in regulating food intake, with defects in this gene being associated with eating disorders. The encoded protein is also involved in a pathway that protects neuroblastoma cells from chemotherapy-induced cell death, providing a possible therapeutic target against neuroblastoma. Diseases associated with NPY5R include Cocaine Dependence and Panic Disorder. Cocaine Dependence is related to cocaine abuse and personality disorder. An important gene associated with Cocaine Dependence is DRD2 (Dopamine Receptor D2), and among its related pathways/superpathways are Peptide ligand-binding receptors and Transmission across Chemical Synapses. The drugs caffeine and Lamotrigine have been mentioned in the context of this disorder. Affiliated tissues include brain, prefrontal cortex and cortex, and related phenotypes are behavior/neurological and homeostasis/metabolism. Panic disorders relate to a group of mental illnesses that involve long-term patterns of thoughts and behaviors that are unhealthy and inflexible. In an embodiment, the enhancer element targets VIP interneuron cells expressing the target gene Grpr (Gastrin-Releasing Peptide Receptor). The Grpr gene product regulates numerous functions of the gastrointestinal and central nervous systems, including release of gastrointestinal hormones, smooth muscle cell contraction, and epithelial cell proliferation and is a potent mitogen for neoplastic tissues. Diseases associated with GRPR include agoraphobia and prostate cancer.

In another aspect, a computer-implemented method for cell type-specific enhancer identification is provided, in which the method involves the steps of a) quantifying peaks by analyzing single cell Assay of Transposase Accessible Chromatin sequencing (scATAC-seq) data to call peaks present in cell subtypes; b) analyzing the quantified peaks present in the scATAC-seq data of a) to identify cell type specific enhancers; c) assigning cell type identity comprising integrating the scATAC-seq data of b) and single-cell RNA-seq (scRNA-seq) data, wherein marker expression patterns in the scRNA-seq data provide for the assignment of cell type identity for cells in the scATAC-seq data; and d) validating cell type specific enhancers comprising identifying regions of conservation across species in cell type-specific accessibility patterns present in cell type specific enhancers. In an embodiment, prior to peak quantification, the method comprises quantifying the accessibility of bins of genomic regions and projecting the bins into a cell space. In an embodiment, the bins comprise genomic regions of approximately 500 base pairs (bp). In an embodiment, the method further involves comparing each bin to an ideal cell type-specific bin to prioritize sub-type specific bins that most closely resemble the ideal cell type-specific bin. In an embodiment, the prioritized sub-type specific bins are used in the peak quantification of step (a) of the method delineated above. In an embodiment, the peaks are merged with peaks called from individual cell types and peaks called from all cells to generate a union peak set. In an embodiment, the union peak set is used to determine cell population heterogeneity prior to carrying out step (b) of the above-delineated method. In an embodiment, the scRNA-seq and scATAC-seq data are obtained from one or more neurons or interneurons. In an embodiment, the neurons are present in brain cortex or cortical tissue. In an embodiment, the neurons or interneurons are somatostatin (SST)-expressing interneurons, inhibitory interneurons, Parvalbumin (PV)-expressing basket cells, PV-expressing chandelier interneurons, Vasoactive intestinal polypeptide (VIP)-expressing interneurons, or Lamp5-expressing interneurons. In an embodiment, the method further involves the step of displaying or translating the results or output of the steps in a visual form. In an embodiment, the step of displaying or translating includes displaying the results or output on a display device. In an embodiment, the display device is a desktop computer, a laptop computer, a hand-held computer, a smart phone, a cellular telephone, a tablet computer, or a personal digital assistant. In an embodiment of the above-delineated method and/or embodiments thereof, the method further comprises isolating or cloning the cell type-specific enhancer sequence identified by the method.

In another aspect, a cell type-specific enhancer sequence identified by the above-delineated computer-implemented method and/or embodiments thereof is provided. In an embodiment, the cell type-specific enhancer sequence identified by the method comprises a polynucleotide sequence selected from any one of SEQ ID NOs: 1-16.

### DEFINITIONS

Unless defined otherwise, all technical and scientific terms used herein have the meaning commonly understood by a person skilled in the art to which the described aspects and embodiments belong. The following references provide one of skill with a general definition of many of the terms used in the described embodiments: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “administering” is meant giving, supplying, dispensing a composition, agent, therapeutic product, e.g., a virus vector (rAAV) harboring a transgene (e.g., an effector gene or polynucleotide, a therapeutic gene or polynucleotide, or a gene or polynucleotide encoding an effector nuclease), and the like to a subject, or applying or bringing the composition and the like into contact with the subject. Administering or administration may be accomplished by any of a number of routes, such as, for example, and without limitation, parenteral or systemic, intravenous (IV injection), subcutaneous (SC), intrathecal (IT), intracranial (IC), intramuscular (IM), dermal, intradermal (ID), inhalation, rectal, intravaginal, topical, oral, subcutaneous, intramuscular, or intraocular. In embodiments, administration is systemic, such as by inoculation, injection, or intravenous injection.

By “agent” is meant a peptide, polypeptide, nucleic acid molecule, or small molecule chemical compound, antibody, or a fragment thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels. An alteration may reflect a change based on a reference or a control. In some embodiments, the reference or control can include a normal, standard, or customary expression level or activity of a gene or polypeptide. In some embodiments, the reference or control can be an abnormal, diseased, nonstandard, or noncustomary expression level or activity of a gene or polypeptide.

By “ameliorate” and “amelioration” is meant decrease, suppress, attenuate, diminish, abate, arrest, or stabilize the development or progression of a disease.

By “analog” or “derivative” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, polynucleotide binding activity. In another example, a polynucleotide analog retains the biological activity of a corresponding naturally-occurring polynucleotide while having certain modifications that enhance the analog's function relative to a naturally occurring polynucleotide. Such modifications could increase the polynucleotide's affinity for DNA, half-life, and/or nuclease resistance, an analog may include an unnatural nucleotide or amino acid.

The term “Assay of Transposase Accessible Chromatin sequencing (ATAC-seq)” refers to an art-recognized method used in the study and analysis of chromatin biology. The chromatin analysis can be performed at the level of single cells and is referred to as “single cell Assay of Transposase Accessible Chromatin sequencing (scATAC-seq).” In brief, ATAC-seq incorporates a genetically engineered hyperactive Tn5 transposase that simultaneously cuts open chromatin leaving a 9-bp staggered nick and ligates high-throughput sequencing adapters to these regions. During this process, the nick is repaired, leaving a 9-bp duplication. Paired-end sequencing is then performed to facilitate higher unique alignment rates of these open chromatin regions. A review of ATAC-seq and data analysis resulting therefrom may be found in F. Yan et al., 2020, Genome Biology, 21 (22): 1-16. See also, R. Fang et al., 2021, Nature Communications, 12 (1337): 1-15.

The term “binning,” also known as discretization or bucketing, is a data preprocessing technique used in data mining. It involves dividing a number of continuous values or variables into a set of smaller intervals or “bins” and replacing the original values with the corresponding bin labels. Binning can be applied to both numerical and categorical variables; its primary purpose is to simplify the data and make it more manageable for analysis. By way of example, binning in data mining can be used to discretize a numerical variable, such as age, into age groups (e.g., 0-18, 19-30, 31-50, and 51+), which can be useful for analysis and modeling purposes. Binning can be useful for reducing complexity of data by grouping values into smaller number of categories or intervals, making the data easier to understand, summarize, and visualize; for reducing the noise in the data by smoothing out variations in individual data points and highlighting larger patterns, improving the accuracy of predictive models, and making the data easier to understand and interpret.

In metagenomics, the assignment of genomic sequence fragments to a corresponding taxonomic group, e.g. cell, species, genera or higher taxonomic group, is referred to as “binning” as each of the sequence fragments is placed into a bin or group, which ideally represents only sequence fragments belonging to that group. Binning can be performed after assembly of raw sequence reads into contigs and involves the clustering of those contigs that belong together, e.g., constitute a (partial) genome of a single population or cell type (or of a group of closely related populations or cells). When the sequencing coverage is sufficiently high and when the “microdiversity” is not too high, the resulting bins can be considered provisional whole-genome-sequences of the source populations. A bin, which is equivalent to a peak in a trace curve, holds data values around a local maximum of a single sample. It has a certain width in data points and, after sizing, also a width in base sizes. A bin comprises data points with the highest data points usually forming a local maximum near the center. The sum of all data point values is the area.

“Peak calling” refers to a computational method used to identify areas in a genome that have been enriched with aligned reads as a result of performing a ChIP (chromatin immunoprecipitation)-sequencing or a MeDIP-seq (Methylated DNA immunoprecipitation-sequencing) experiment. ChIP-sequencing, also known as ChIP-seq, is a method used to analyze protein interactions with DNA. ChIP-seq combines chromatin immunoprecipitation (ChIP) with massively parallel DNA sequencing to identify the binding sites of DNA-associated proteins. Such areas are those at which a protein interacts with DNA. It can be used to map precisely global binding sites for any protein of interest. For ChIP-seq, the alignment of the reads to the genome results in two peaks (one on each strand) that flank the binding location of the protein or nucleosome of interest. In general, peaks are regions of the genome where there is an enrichment of reads compared to the background. Factors that are used to assess peak significance are the fraction of reads in peaks and the number of reads in peaks.

As used herein, the term “at risk” as it applies to a neurological, neurodevelopmental, neurodegenerative, neuropsychiatric, neurogenetic, or neuromuscular disease, disorder, or pathology, such as seizures or epilepsy, refers to patients or individuals who have a family history or genetic risk factor genes for a neurological, neurodevelopmental, neurodegenerative, neuropsychiatric, neurogenetic, or neuromuscular disease, disorder, or pathology, and/or symptoms thereof. Such patients or individuals may be considered to be susceptible to the aforementioned disease, disorder, pathology, and/or the symptoms thereof.

As used herein, the term “carrier” refers to a diluent, adjuvant, excipient, or vehicle with which a composition or pharmaceutical composition, e.g., comprising a polynucleotide, viral vector, or viral particle) can be administered. Pharmaceutical and pharmaceutically acceptable carriers include sterile liquids, such as water and oils, including those of petroleum, animal, vegetable or synthetic origin, such as peanut oil, soybean oil, mineral oil, sesame oil, and the like. Water or aqueous saline solutions and aqueous dextrose and glycerol solutions may be employed as carriers, particularly for injectable solutions. Carriers may also include solid dosage forms, including, but not limited to, one or more of a binder (for compressed pills), a glidant, an encapsulating agent, a flavorant, and a colorant. Suitable pharmaceutical carriers are described in “Remington's Pharmaceutical Sciences” by E. W. Martin.

A “computer readable medium” refers to any article of manufacture that contains data that can be read by a computer (non-transitory media) or a carrier wave signal carrying data that can be read by a computer. Such computer readable media include, without limitation, magnetic media, such as a disk, tape, or cards; optical media such as CD-ROM or writeable compact disk; magneto-optical media in disk, tape, or card form; paper media, such as punched cards or paper tape; or on carrier wave signal received through a network, wireless network or modem, internet, including, but not limited to, radio-frequency signals, satellite signals, and infrared signals.

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

“DREADD” is an acronym for “designer receptor exclusively activated by a designer drug,” which is a modified G protein coupled receptor (GPCR) that may be administered or specifically introduced into a subject, or cells thereof, e.g., PV-expressing interneurons, by use of a viral vector (which contains a polynucleotide sequence encoding the DREADD) or through genetic breeding. DREADDs, which are known as chemical genetic or “chemogenetic” molecules, allow for a precise level of temporal control over the excitation and inhibition of neurons. Following expression of the DREADD, it may be activated by a specific ligand (or agonist), which may be administered by intravenous injection or orally. The DREADD and its ligand are designed to be orthogonal, i.e., they bind specifically to each other and do not cross-react. By way of nonlimiting example, five different classes of DREADDs are available for use: hM3Dq raises calcium levels in a cell, causing burst firing; hM4Di lowers cAMP and the activation of a particular potassium channel, causing neuronal silencing, and also inhibits presynaptic neurotransmitter release; GsD enhances cAMP, causing modulation signaling; and Rq (R165L) enhances arrestin signaling, a specific pathway that has been linked to the mechanisms of psychoactive drugs; and K-opioid receptor DREADD or KORD, which reduces or inhibits excitation of neurons and also inhibits presynaptic neurotransmitter release. (See, e.g., Kelly Rae Chi, 2015, The Scientist, and S. M. Sternson and B. L. Roth, 2014, Ann Rev Neuroscience, 37:387-407). DREADDs have been successfully expressed in nonhuman primates without apparent toxicity, and CNO-DREADDs can modulate circuitry, electrophysiology, and behavior in nonhuman primates (M. A. G. Eldridge et al., 2016, Nature Neurosci., 19:37-39). In an embodiment, the chemical actuator perlapine, an approved medication in humans may be used as a DREADD ligand for activating CNO-based DREADDs in humans. Other small molecule DREADD actuators may include salvinorin B and/or its precursor, salvinorin A, (See, e.g., B. L. Roth, 2016, Neuron, 89 (4): 683-694).

Orthogonal ligand-gated ion channels, called pharmacologically selective actuator molecules (PSAMs) and pharmacologically selective effector molecules (PSEMs), are other types of chemogenetic molecules that are used as optogenetic agents and in optogenetic methods, in a manner similar to the use of DREADDs. Each PSAM is exclusively activated by a PSEM cognate synthetic agonist. By way of nonlimiting example, three specific PSAM/PSEM tools have been designed, each with different ion conductance properties for controlling neuronal excitability. (See, e.g., Shapiro, M. G. et al., 2012, ACS Chem. Neurosci., 3 (8): 619-629). These include the cation-selective activator, PSAM^{Q79G,Q139G}-5HT3HC/PSEM²²⁵, the anion-selective silencer, PSAM^{L141F,Y115F}-GlyR/PSEM^{89S}, and a third Ca²⁺-selective channel, PSAM^{Q79G,L141S}-nAChR V13′T/PSEM^{9S}. (See, Ibid., and Magnus, C. J. et al., 2011, Science, 333 (6047): 1292-1296). Both DREADDS and PSAMs-PSEMs allow control over neuronal activity, in a temporal manner, from minutes to hours. (See, e.g., Kelly Rae Chi, 2015, The Scientist; and S. M. Sternson and B. L. Roth, 2014, Ann Rev Neuroscience, 37:387-407). By way of example, different PSAMs have been used with various ion channels and PSEMs to control neurons, e.g., E/I balance in neurons. Such PSAM-PSEM pairings include, without limitation, PSAM^{L141F,Y115F}-5HT3 HC, which is activated by the ligand PSEM^{89S}, allowing cations to flow into the cell and boost excitability; PSAM^{L141F, Y115F}-GlyR, which is activated by the ligand PSEM^{89S}, silencing neurons; and PSAM^{Q79G, L141S}-nAChR V13, which is activated by the ligand PSEM^{9S}, enhancing calcium signaling. Because there are two different PSEM ligands, PSAMs-PSEMs can also be combined in the same animal (subject).

“Detect” refers to identifying the presence, absence or amount of a molecule, compound, or agent to be detected.

By “disease” is meant any pathology, disorder, condition, and/or the symptoms thereof, that adversely affects, damages or interferes with the normal function of a cell, tissue, organ, or part of the body, such as the brain, including the cerebral cortex of the brain and brain tissues, or central nervous system (CNS). In embodiments, the disease is a developmental or neurodevelopmental disorder, a neuropathological disorder, neuropsychological disorder, a neuronal disorder, a neuromuscular disorder, a neurophysiological disorder, and/or the symptoms thereof. In an embodiment, a symptom of a disease, pathology, or disorder, such as a neuropsychiatric disorder, is seizures. Nonlimiting examples of other neurological, neurodevelopmental, neurodegenerative, neuropsychiatric, neurogenetic, or neuromuscular diseases, disorders, or pathologies include Alzheimer's disease, Parkinson's disease, Dystonia, ALS and Down Syndrome.

By “effective amount” is meant the amount of a required to ameliorate the symptoms of a disease relative to an untreated patient. The effective amount of active compound(s) used to practice the described methods for therapeutic treatment of a disease varies depending upon the manner of administration, the age, body weight, and general health of the subject. Ultimately, the attending physician, clinician, or veterinarian will decide the appropriate amount and dosage regimen. Such amount is referred to as an “effective” amount. In one embodiment, an effective amount is the amount of an rAAV vector comprising a specific enhancer sequence (e.g., such as ME1-ME8, or the human orthologs thereof, of SEQ ID NOs: 1-16 as described herein) and one or more transgene sequences having an activity or function following expression in a given cell. By way of nonlimiting example, a therapeutic gene sequence inserted in the vector can reduce, ameliorate, abate, inhibit, eliminate, or stabilize a symptom of a neurological, neurodevelopmental, neurodegenerative, neuropsychiatric, neurogenetic, or neuromuscular disease or disorder, or the severity thereof following expression in the cell. In another embodiment, an effective amount is the amount of an rAAV vector comprising a specific enhancer sequence (e.g., ME1-ME8, or the human orthologs thereof, of SEQ ID NOs: 1-16 as described herein) and one or more transgenes (e.g., a therapeutic gene, or effector genes encoding products such as Gq-DREADD or PSAM, or a clustered regularly interspaced short palindromic repeats-Cas9 (CRISPR-Cas9) protein or variant thereof, a Zinc Finger Protein, a Transcription activator-like effector nuclease (TALEN), or an engineered form thereof) sequences inserted therein required to direct, restrict, or regulate expression of the transgene in a target neuron cell type or population, such as a GABA-ergic interneuron cell, for example, parvalbumin (PV)-expressing interneurons, e.g., SEQ ID NOs: 1, 9, 2 and 10; Somatostatin (SST)-expressing interneurons, e.g., SEQ ID NOs: 3 and 11; Vaso-active Intestinal Peptide (VIP)-expressing interneurons, e.g., SEQ ID NOs: 8 and 16; and Lamp5-expressing interneurons, e.g., SEQ ID NOs: 4-7 and 12-15. In embodiments, the enhancer element sequence is an isolated and cloned mouse enhancer sequence or the human ortholog of the mouse enhancer sequence as described herein.

As used herein, the term “endogenous” describes a molecule (e.g., a polypeptide, peptide, nucleic acid, or cofactor) that is found naturally in a particular organism (e.g., a human) or in a particular location within an organism (e.g., an organ, a tissue, or a cell, such as a human cell).

As used herein, the term “exogenous” refers to a molecule (e.g., a polypeptide, peptide nucleic acid, or cofactor) that is not found naturally or endogenously in a particular organism (e.g., a human) or in a particular location within an organism (e.g., an organ, a tissue, or a cell, such as a human cell). Exogenous materials include those that are provided from an external source to an organism or to cultured matter extracted therefrom.

An “enhancer,” “enhancer element,” “enhancer sequence,” or “enhancer regulatory element or sequence,” refers to a nucleic acid or polynucleotide sequence or a region of a nucleic acid or polynucleotide sequence, e.g., DNA or RNA, of about 50-2500 nucleotides, that contains one or more binding sites that are recognized and bound by one or more binding protein(s), e.g., transcription factor(s). In general, the binding proteins function as activators to increase the likelihood that transcription of a particular target gene will occur. Enhancers can activate transcription independent of their location, distance or orientation with respect to the promoters of genes. For example, enhancer sequences may be located upstream of a gene, downstream of a gene, within the coding region of a gene, or up to one million base pairs away from the gene. Typically, and without intending to be bound by theory, the binding of a DNA binding protein(s) or transcription factor(s) to an enhancer changes or alters the conformation of the DNA, thereby allowing interactions to occur between or among the transcription factor(s) bound to the DNA.

Enhancers have been described as clusters of DNA sequences capable of binding combinations of transcription factors that then interact with components of the mediator complex or TFIID to help recruit RNA polymerase II (RNAPII). To accomplish this, enhancer-bound transcription factors loop out the intervening sequences and contact the promoter region of a gene, thus allowing enhancers to act in a distance-independent fashion. In addition, activation of eukaryotic genes requires de-compaction of the chromatin fiber, which is carried out by enhancer-bound transcription factors that can recruit histone modifying enzymes or ATP-dependent chromatin remodeling complexes to alter chromatin structure and increase the accessibility of the DNA to other proteins. (For a review of enhancer function, see, e.g., Ong, C.-T. and Corces, V. G., 2011, Nat. Rev. Genetics, 12 (4): 283-293).

As described herein, mouse enhancer sequences and their human counterpart (ortholog) sequences were identified for targeting intended neuronal cells and/or neuronal cell populations. The isolated, cloned enhancer sequences, called ME1-ME8 having SEQ ID NOs: 1-8 and their human counterparts huE1-HuE8 having SEQ ID NOs: 9-16, respectively, as described herein, were discovered to have the ability to target, restrict, or regulate the expression of a transgene (e.g., an exogenous polynucleotide sequence encoding a target protein, such as a therapeutic protein or reporter protein) or an effector gene, within certain neuronal cell types, such as inhibitory GABA-ergic interneurons (e.g., parvalbumin (PV)-expressing interneurons, e.g., PV-expressing basket or chandelier cells; Somatostatin (SST)-expressing interneurons; Vaso-active Intestinal Peptide (VIP)-expressing interneurons; and Lamp5-expressing interneurons). In an embodiment, the enhancer element is isolated from a cell, such as a neuron or interneuron or subtype thereof. Such an enhancer element is molecularly cloned, isolated, and used or contained in a delivery vector, e.g., a viral vector, for delivery to a cell, tissue, or region of the body, such as the brain or central nervous system (CNS).

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “functionally expressed” is meant that a gene, polynucleotide, or transgene contained in or inserted into the polynucleotide of an rAAV or rAAV vector as described herein is expressed in an infected or transduced cell and produces its encoded product, which is functional and/or active in the cell. In an embodiment, the cell is an interneuron cell. In an embodiment, the cell is a GABA-ergic interneuron cell. In an embodiment, the gene or polynucleotide of interest is a therapeutic gene, which encodes a therapeutic protein, or an effector protein, such as a nuclease, etc. as described herein. In an embodiment, the gene or polynucleotide of interest is a detectable reporter gene, such as d-Tomato (excitation peak: 554 nm; emission peak: 581 nm; a fluorescent dimer protein that emits orange-red light when it is excited by green-yellow light), Channelrhodopsin (ChR2), Green Fluorescent Protein (GFP), Red Fluorescent Protein (RFP), and the like. In an embodiment, the gene or polynucleotide of interest encodes a Designer receptor exclusively activated by designer drugs (DREADD) or Gq-DREADD. In an embodiment, the gene or polynucleotide of interest encodes PSAM. In embodiments, the gene or polynucleotide of interest encodes a clustered regularly interspaced short palindromic repeats-Cas9 (CRISPR-Cas9) protein or variant thereof, a Zinc Finger Protein, a Transcription activator-like effector nuclease (TALEN), or an engineered form thereof. Cas9 is a dual RNA-guided endonuclease enzyme associated with the Clustered Regularly Interspaced Short Palindromic Repeats (CRISPR) adaptive immune system in bacteria such as, without limitation, Streptococcus pyrogenes. Cas9 uses base pairing to recognize and cleave target DNAs with complementarity to the guide RNA. The programmable sequence specificity of Cas9 and RNA-guided, DNA cleavage or nicking activities of Cas9 have been hamessed for precise genome editing and gene expression control in many cells and organisms. In brief, Cas9 and a guide RNA interact to form a complex that can identify target nucleic acid sequences with high selectivity. Cas9 locates and cleaves or nicks a target DNA in CRISPR/Cas systems. It will be appreciated by those skilled in the art that a guide RNA is included in engineered CRISPR/Cas9 systems to bind to the Cas9 protein and induce a conformational change in Cas9, which promotes its effector function and activity. (M. Jinek et al., 2014, Science, 343 (6176); H. Nishimasu et al., 2014, Cell, 156 (5): 935-949; J. A. Doudna et al., 2014; Science, 346 (6213)). In an embodiment, a guide RNA is used in conjunction with Cas9 as an effector protein encoded by a polynucleotide in a vector harboring an isolated, cloned enhancer element as described herein. Zinc Finger proteins constitute transcriptional activator proteins containing a zinc finger domain structure (i.e., a structural motif characterized by the coordination of one or more zinc ions), which function to bind and interact with DNA, RNA, poly-ADP-ribose, and other proteins or molecules, and play a role in sequence specific gene regulation. (M. Cassandri et al., 2017, Cell Death Discov., 3 (17071); J. H. Laity et al., 2001, Curr. Opin. Struct. Biol., 11:39-46). By way of example, engineered zinc finger arrays can be fused to a DNA cleavage domain (e.g., the DNA cleavage domain of Fok1) to generate zinc finger nucleases. Zinc finger-Fok1 fusions are useful for sequence-specific manipulation of genomes in mammalian cells and organisms. (B. Schierling et al., 2012, Nucleic Acids Research, 40 (6): 2623-38; J. Guo et al., 2010, J. Mol. Biol., 400 (1): 96-107). TALENs, which are nucleases that contain DNA binding domains, are useful as genome editing tools for targeted gene editing and genome modifications, e.g., by creating a targeted double-strand break in the genome (or cellular DNA) that stimulates cellular DNA repair through homology directed repair (HDR) or non-homologous end-joining (NHE). (J. C. Miller et al., 2010, Nature Biotechnology, 29, 143-148; D. G. Ousterout et al., 2016, Methods Mol. Biol., 1338:27-42). TALENs can be generated by fusing a (designed) DNA binding domain that recognizes a specific DNA sequence to a nonspecific DNA cleaving domain, allowing for the cleavage of DNA at a specific site with high accuracy.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The term “interneuron” refers to a neuron (nerve cell), or local circuit neuron in the central nervous system (CNS) that relays impulses between sensory neurons and motor neurons. In general, neurons are specialized cells that function primarily in the transmission of nerve impulses. Neurons have cellular processes, such as dendrites and axons. Dendrites, which are shorter processes in the cell body of a neuron, receive inputs from other neurons and conduct signals to the cell body. Axons are longer, single processes of the cell soma and relay signals toward the tip of the neuron (called the synaptic terminal). The three, main types of neurons include sensory neurons, interneurons (of the CNS), and motor neurons. In the human brain, there are about 100 billion interneurons, which receive impulses from the sensory neurons. Interneurons interpret the information received from other neurons and relay impulses to motor neurons for an appropriate response in a function called ‘integration.’ The term “neuron” may be used interchangeably herein with the term “interneuron,” which may be considered to be a neuron that exhibits a certain type of activity, as described supra.

The terms “isolated,” “purified,” or “biologically pure” refer to material that is free to varying degrees from components which normally accompany or are associated with it as found in its native state. “Isolate” denotes a degree of separation from original source or surroundings. “Purify” denotes a degree of separation that is higher than isolation. A “purified” or “biologically pure” protein or polynucleotide is sufficiently free of other materials such that any impurities do not materially affect the biological properties of the protein or polynucleotide, or cause other adverse consequences. That is, a polynucleotide (nucleic acid), polypeptide, or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid, protein, or peptide gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which flank the gene in the naturally-occurring genome of the organism from which a nucleic acid molecule, such as a nucleic acid molecule described herein, is derived. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, a polypeptide is isolated when it is at least 60%, by weight, free from the proteins and naturally-occurring organic molecules with which it is naturally associated. Preferably, the preparation is at least 75%, or at least 85%, or at least 90%, or at least 99%, by weight, a desired polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression, level or activity that is associated with a disease or disorder.

The term “mutation,” as used herein, refers to a substitution of a nucleotide base or amino acid residue within a sequence, e.g., a nucleic acid or amino acid sequence, respectively, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

As used herein, “obtaining” as in “obtaining an agent” includes synthesizing, purchasing, or otherwise acquiring the agent.

By “polynucleotide” is meant a nucleic acid molecule, e.g., a double-stranded (ds) DNA polynucleotide, a single-stranded (ss) DNA polynucleotide, a dsRNA polynucleotide, or a ssRNA polynucleotide, that encodes one or more polypeptides. The term encompasses positive-sense (i.e., protein-coding) DNA polynucleotides, which are capable of being transcribed to form an RNA transcript, which can be subsequently translated to produce a polypeptide following one or more optional RNA processing events (e.g., intron excision by RNA splicing, or ligation of a 5′ cap or a 3′ polyadenyl tail). The term additionally encompasses positive-sense RNA polynucleotides, capable of being directly translated to produce a polypeptide following one or more optional RNA processing events. As used herein, a polynucleotide may be contained within a viral vector, such as a recombinant adeno-associated viral vector (rAAV).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (2′-e.g., fluororibose, ribose, 2′-deoxyribose, arabinose, and hexose); and/or modified phosphate groups (e.g., phosphorothioates and 5′-N-phosphoramidite linkages).

As used herein, the term “pharmaceutically acceptable” refers to molecular entities, biological products and compositions that are physiologically tolerable and do not typically produce an allergic or other adverse reaction, such as gastric upset, dizziness and the like, when administered to a patient (e.g., a human patient).

As used herein, the terms “prevent,” “preventing,” “prevention,” “prophylactic treatment” and the like refer to reducing the probability of developing a disorder or condition in a subject, who does not have, but who is at risk of, susceptible to, or predisposed to, developing a disorder or condition.

As used herein, the term “pseudotyped” refers to a viral vector that contains one or more foreign viral structural proteins, e.g., envelope glycoproteins. A pseudotyped virus may be one in which the envelope glycoproteins of an enveloped virus or the capsid proteins of a non-enveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. Examples of pseudotyped viral vectors include a virus that contains one or more envelope glycoproteins that do not naturally occur on the exterior of the wild-type virus. Pseudotyped viral vectors can infect cells and express and produce proteins or molecules encoded by polynucleotides, e.g., reporter or effector proteins or molecules, contained within the viral vectors.

The term “recombinant” as used herein in the context of proteins or nucleic acids refers to proteins or nucleic acids that do not occur in nature (or in a naturally occurring protein or nucleic acid sequence), but are the product of human engineering, often or typically utilizing molecular biological or molecular genetic tools and techniques practiced by the skilled practitioner in the art. For example, in some embodiments, a recombinant protein or nucleic acid molecule comprises an amino acid or nucleotide sequence that comprises at least one, at least two, at least three, at least four, at least five, at least six, at least seven, or at least eight mutations as compared to any naturally occurring sequence.

By “reduces” is meant a negative alteration of at least 5%, 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence, for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, or about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, or about 100 nucleotides, or about 300 nucleotides, or any integer thereabouts or therebetween.

By “specifically binds” is meant a nucleic acid molecule, polypeptide, or complex thereof (e.g., a binding protein such as a transcription factor and its cognate nucleic acid binding region), or a compound, or molecule that recognizes and binds a given polypeptide and/or nucleic acid molecule, but which does not substantially recognize and bind other molecules in a sample, for example, a biological sample.

By “subject” is meant a mammal, including, but not limited to, a human or non-human mammal, such as a non-human primate, e.g., a marmoset, or a non-human mammal, such as a bovine, equine, canine, ovine, or feline mammal, or a sheep, goat, llama, camel, or a rodent (rat, mouse), ferret, gerbil, hamster, or zebrafish. A subject is typically a patient, such as a human patient, who receives treatment for a particular disease or condition as described herein (e.g., neurological, neurodevelopmental, neurodegenerative, neuropsychiatric, neurogenetic, or neuromuscular disease, disorder, or pathology, such as seizures or epilepsy), and/or the symptoms thereof. Examples of subjects and patients include mammals, such as humans, receiving treatment for such diseases, pathologies, or conditions, or who are at risk of, or susceptible to, having such diseases, pathologies, or conditions.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

As used herein, the term “therapeutically effective amount” refers to a quantity of a therapeutic agent that is sufficient to treat, abate, reduce, diagnose, prevent, and/or delay the onset of one or more symptoms of a disease, pathology, disorder, and/or condition upon administration to a patient in need of treatment. In some cases, a therapeutically effective amount may also refer to a quantity of a therapeutic agent that is administered prophylactically (e.g., in advance of the development of full-blown disease) to a subject who is at risk of, or susceptible to, developing a disease, pathology, or condition, or the symptoms thereof, such as a neurological, neurodevelopmental, neurodegenerative, neuropsychiatric, neurogenetic, or neuromuscular disease, pathology, disorder, or condition. In an embodiment, a symptom of a disease, pathology, or disorder, such as a neuropsychiatric disorder, is seizures. Nonlimiting examples of other neurological, neurodevelopmental, neurodegenerative, neuropsychiatric, neurogenetic, or neuromuscular diseases, disorders, or pathologies include Alzheimer's disease, Parkinson's disease, Dystonia, ALS and Down Syndrome.

A “therapeutic gene,” which may be a transgene harbored in a vector with an enhancer element sequence as described herein, refers to a polynucleotide sequence (gene) that encodes a therapeutic protein or molecule. Without limitation, a therapeutic gene (or transgene) may constitute a normal, functional gene, or functional portion thereof, for expression in a particular cell type, such as a neuronal or interneuronal cell types described herein, to correct cellular defects and ameliorate, abate, abrogate, alleviate, or eliminate (cure) a disease, such as a neurological, neurodevelopmental, neurodegenerative, neuropsychiatric, neurogenetic, or neuromuscular disease, disorder, or pathology, and/or the symptoms thereof, as described herein. (See, e.g., T. Friedman et al., 1989, Science, Vol. 22 (4910): 1275-1281). By way of example, in delivery vectors containing an enhancer element sequence (e.g., one or more of SEQ ID NOs: 1-16) and a therapeutic gene, the expression of a therapeutic gene is restricted (or regulated) by the presence of the enhancer element to certain neuronal or interneuronal cell types and/or populations transduced by the vector. In such cases, the expressed therapeutic gene can replace a faulty, abnormal or aberrant gene or provide a new gene or can encode a therapeutic protein whose expression restores function, restores normal function, compensates for, and/or improves or abrogates abnormal function of a neuronal or interneuronal cell-expressed protein or polypeptide, in an attempt to correct or cure a disease or disorder.

As used herein a “transgene” refers to an exogenous gene or polynucleotide, i.e., a gene or polynucleotide of interest, that encodes a protein or polypeptide and is introduced into and expressed in a neuronal cell type or population, by a number of genetic engineering techniques or methods, including, but not limited to, an expression vector such as an rAAV vector or other vector. The vector typically contains, in addition to an enhancer sequence as described, other regulatory sequences, such as, for example, a promoter sequence to drive protein expression and a polyadenylation signal. By way of non-limiting example, transgenes include reporter genes, therapeutic genes, effector genes such as DREADD- and PSAM-encoding “effector” genes, or another type of “effector” gene that can be used to edit a gene or a polynucleotide sequence (e.g., a protein-encoding polynucleotide sequence; a target gene or polynucleotide sequence), and/or modulate or edit the expression of a target gene or polynucleotide sequence, for example, polynucleotides encoding CRISPR-Cas9 proteins, Zinc Finger Proteins, engineered Zinc Finger Proteins, and Transcription activator-like effector nucleases (TALENs), which are restriction enzymes that can be engineered to cut specific sequences of DNA. TALENs are made by fusing a TAL effector DNA-binding domain to a DNA cleavage domain (a nuclease that cuts DNA strands). Transcription activator-like effectors (TALEs) can be engineered to bind to practically any desired DNA sequence so that when combined with a nuclease, DNA can be cut at specific locations. The restriction enzymes can be introduced into cells, e.g., for use in gene editing, gene therapy, or for genome editing in situ, e.g., employing genome or base editing with engineered nucleases. Zinc finger nucleases, CRISPR/Cas9, and variants and analogs thereof, as well as TALENs are useful in the field of genome or base editing.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. “Treat” or “treatment” may refer to therapeutic treatment, in which the object is to prevent or slow down (lessen or reduce) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in whom the condition or disorder is to be prevented.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As used herein, the term “vector” refers to a nucleic acid (e.g., a DNA vector, such as a plasmid), an RNA vector, virus or other suitable replicon (e.g., viral vector). A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a transgene, such as a therapeutic gene, a reporter gene, or an effector gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site in order to direct efficient transcription of a gene carried on the expression vector. Vectors, such as viral vectors or the rAAV vectors described herein, may also be referred to as expression vectors. In an embodiment, the components, polynucleotides, or encoding polynucleotides contained in a vector are operably linked to allow for expression in a cell.

“Transduction” refers to a process by which DNA or polynucleotide, e.g., one or more transgenes, contained in a virus or virus vector is introduced or transferred into a cell by the virus or virus vector, wherein the DNA or polynucleotide is expressed. In an embodiment, the DNA or polynucleotide transduced into a cell by a virus vector, such as an rAAV vector as described herein, is stably expressed in the cell. In some cases, a virus or virus vector is said to infect a cell.

As used herein, the term “vehicle” refers to a solvent, diluent, or carrier component of a pharmaceutical composition.

By “virus particle” (also called a virion) is meant a virus (infectious agent) that exists as an independent particle comprising the core viral genome or genetic material (RNA or DNA); a protein coat, called the capsid, which surrounds the genetic material and protects it; and, in some cases, an envelope of lipids surrounding the capsid. A virus particle may refer to the form of a virus before it infects a cell and becomes intracellular, or to the form of the virus that infects a cell.

By “virus-like particles (VLPs)” is meant virus particles made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 70%, at least 75%, at least 80%, at least 85%, at least 90%, at least 93%, at least 95%, or greater, at least 98%, or greater, or at least 99%, or greater, identical at the amino acid level or nucleic acid level to the sequence used for comparison, for example, over a specified comparison window. Optimal alignment may be conducted using the homology alignment algorithm of Needleman and Wunsch, 1970, J. Mol. Biol., 48:443. An indication that two peptide or polypeptide sequences are substantially identical is that one peptide or polypeptide is immunologically reactive with specific antibodies raised against the second peptide or polypeptide, although such cross-reactivity is not required for two polypeptides to be deemed substantially identical. Thus, a peptide or polypeptide is substantially identical to a second peptide or polypeptide, for example, where the two differ only by a conservative substitution. Peptides or polypeptides that are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person in the art.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

Polynucleotides or viral nucleic acid molecules useful in the methods and compositions as described herein include any nucleic acid molecule that encodes a polypeptide, or a fragment thereof, or that encodes the components of viral vectors described herein. The polynucleotides or viral nucleic acid molecules may encode polypeptide products harbored by the viral vectors, such as recombinant adeno-associated virus (rAAV) and the like, as well as a peptide or fragment thereof. Such nucleic acid molecules need not be 100% identical with an endogenous sequence or a viral vector nucleic acid sequence, but will typically exhibit substantial identity. Polynucleotides having substantial identity to an endogenous sequence or to a viral vector sequence are typically capable of hybridizing with at least one strand of a double-stranded nucleic acid molecule or to a viral vector nucleic acid molecule. Nucleic acid molecules useful in the described methods include any nucleic acid molecule that encodes a polypeptide as described herein, or a fragment thereof. By “hybridize” is meant pairing or the nucleic acid molecules to form a double-stranded molecule between complementary polynucleotide sequences (e.g., a gene or nucleic acid sequence described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30 °C, more preferably of at least about 37 °C, and most preferably of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30 °C in 750 mM NaCl, 75 mM trisodium citrate, and 1% SDS. In a more preferred embodiment, hybridization will occur at 37 °C in 500 mM NaCl, 50 mM trisodium citrate, 1% SDS, 35% formamide, and 100 μg/ml denatured salmon sperm DNA (ssDNA). In another embodiment, hybridization will occur at 42 °C in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 °C, more preferably of at least about 42 °C, and even more preferably of at least about 68 °C. In an embodiment, wash steps will occur at 25 °C in 30 mM NaCl, 3 mM trisodium citrate, and 0.1% SDS. In another embodiment, wash steps will occur at 42 °C in 15 mM NaCl, 1.5 mM trisodium citrate, and 0.1% SDS. In yet another embodiment, wash steps will occur at 68 °C in 15 mM NaCl, 1.5 mM trisodium citrate, and 0.1% SDS. Additional variations of these conditions will be readily apparent to those skilled in the art. Hybridization techniques are well known to those skilled in the art and are described, for example, in Benton and Davis (Science, 196:180, 1977); Grunstein and Hogness (Proc. Natl. Acad. Sci., USA, 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Nonlimiting examples of “moderately stringent hybridization conditions” include a hybridization in a buffer of 40% formamide, 1 M NaCl, 1% SDS at 37 °C, and a wash in 1×SSC at 45 °C. A positive hybridization is at least twice background. Those of ordinary skill will readily recognize that alternative hybridization and wash conditions can be utilized to provide conditions of similar stringency.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e-10, e-20, e-30, e 40, e-50, e-75, e-100. The skilled artisan understands that an ortholog is likely to be functionally related to the reference protein or nucleic acid sequence. In other words, the ortholog and its reference molecule would be expected to fulfill similar, if not equivalent, functional roles in their respective organisms, e.g., mouse and human orthologs. In general, an ortholog is a homologous gene in which the gene sequence has diverged after a speciation event, but the gene and its main function are conserved.

It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs, e.g., human orthologs of mouse sequences, may be identified using methods practiced in the art. The functional role of an ortholog may be assayed using methods well known to the skilled artisan. For example, function might be assayed in vivo or in vitro using a biochemical, immunological, or enzymatic assay; or transformation rescue. Alternatively, bioassays may be carried out in tissue culture; function may also be assayed by gene inactivation (e.g., by RNAi, siRNA, or gene knockout), or gene over-expression, as well as by other methods.

Ranges as provided herein are understood to be shorthand for all of the values within the range. For example, a range of 1 to 50 is understood to include any number, combination of numbers, or sub-range from the group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The amino acid sequence of the human Gq-DREADD (hM3Dq) excitatory receptor is derived from the amino-acid sequence of the unmodified human muscarinic acetylcholine receptor M3 set forth above. In the Gq-DREADD (hM3Dq) receptor amino acid sequence (590 aa), the tyrosine in position 149 is replaced by a cysteine, and the arginine in position 239 is replaced by a glycine (US Publication No. 2018/0078658), as shown below: Met Thr Leu His Asn Asn Ser Thr Thr Ser Pro Leu Phe Pro Asn Ile Ser Ser Ser Trp Ile His Ser Pro Ser Asp Ala Gly Leu Pro Pro Gly Thr Val Thr His Phe Gly Ser Tyr Asn Val Ser Arg Ala Ala Gly Asn Phe Ser Ser Pro Asp Gly Thr Thr Asp Asp Pro Leu Gly Gly His Thr Val Trp Gln Val Val Phe Ile Ala Phe Leu Thr Gly Ile Leu Ala Leu Val Thr Ile Ile Gly Asn Ile Leu Val Ile Val Ser Phe Lys Val Asn Lys Gln Leu Lys Thr Val Asn Asn Tyr Phe Leu Leu Ser Leu Ala Cys Ala Asp Leu Ile Ile Gly Val Ile Ser Met Asn Leu Phe Thr Thr Tyr Ile Ile Met Asn Arg Trp Ala Leu Gly Asn Leu Ala Cys Asp Leu Trp Leu Ala Ile Asp Cys Val Ala Ser Asn Ala Ser Val Met Asn Leu Leu Val Ile Ser Phe Asp Arg Tyr Phe Ser Ile Thr Arg Pro Leu Thr Tyr Arg Ala Lys Arg Thr Thr Lys Arg Ala Gly Val Met Ile Gly Leu Ala Trp Val Ile Ser Phe Val Leu Trp Ala Pro Ala Ile Leu Phe Trp Gln Tyr Phe Val Gly Lys Arg Thr Val Pro Pro Gly Glu Cys Phe Ile Gln Phe Leu Ser Glu Pro Thr Ile Thr Phe Gly Thr Ala Ile Ala Gly Phe Tyr Met Pro Val Thr Ile Met Thr Ile Leu Tyr Trp Arg Ile Tyr Lys Glu Thr Glu Lys Arg Thr Lys Glu Leu Ala Gly Leu Gln Ala Ser Gly Thr Glu Ala Glu Thr Glu Asn Phe Val His Pro Thr Gly Ser Ser Arg Ser Cys Ser Ser Tyr Glu Leu Gln Gln Gln Ser Met Lys Arg Ser Asn Arg Arg Lys Tyr Gly Arg Cys His Phe Trp Phe Thr Thr Lys Ser Trp Lys Pro Ser Ser Glu Gln Met Asp Gln Asp His Ser Ser Ser Asp Ser Trp Asn Asn Asn Asp Ala Ala Ala Ser Leu Glu Asn Ser Ala Ser Ser Asp Glu Glu Asp Ile Gly Ser Glu Thr Arg Ala Ile Tyr Ser Ile Val Leu Lys Leu Pro Gly His Ser Thr Ile Leu Asn Ser Thr Lys Leu Pro Ser Ser Asp Asn Leu Gln Val Pro Glu Glu Glu Leu Gly Met Val Asp Leu Glu Arg Lys Ala Asp Lys Leu Gln Ala Gln Lys Ser Val Asp Asp Gly Gly Ser Phe Pro Lys Ser Phe Ser Lys Leu Pro Ile Gln Leu Glu Ser Ala Val Asp Thr Ala Lys Thr Ser Asp Val Asn Ser Ser Val Gly Lys Ser Thr Ala Thr Leu Pro Leu Ser Phe Lys Glu Ala Thr Leu Ala Lys Arg Phe Ala Leu Lys Thr Arg Ser Gln Ile Thr Lys Arg Lys Arg Met Ser Leu Val Lys Glu Lys Lys Ala Ala Gln Thr Leu Ser Ala Ile Leu Leu Ala Phe Ile Ile Thr Trp Thr Pro Tyr Asn Ile Met Val Leu Val Asn Thr Phe Cys Asp Ser Cys Ile Pro Lys Thr Phe Trp Asn Leu Gly Tyr Trp Leu Cys Tyr Ile Asn Ser Thr Val Asn Pro Val Cys Tyr Ala Leu Cys Asn Lys Thr Phe Arg Thr Thr Phe Lys Met Leu Leu Leu Cys Gln Cys Asp Lys Lys Lys Arg Arg Lys Gln Gln Tyr Gln Gln Arg Gln Ser Val Ile Phe His Lys Arg Ala Pro Glu Gln Ala Leu (SEQ ID NO: 17).

Unless specifically stated or obvious from context, as used herein, the term “or” is understood to be inclusive. Unless specifically stated or obvious from context, as used herein, the terms “a”, “an”, and “the” are understood to be singular or plural.

As used herein, the term “about” or “approximately” means within an acceptable error range for the type of value described and the method used to measure the value. For example, these terms can signify within 20%, more preferably within 10%, and most preferably still within 5% of a given value or range. More specifically, “about” can be understood as within 20%, 10%, 9%, 8%, 7%, 6%, 5%, 4%, 3%, 2%, 1%, 0.5%, 0.1%, 0.05%, or 0.01% of the stated value or range. Alternatively, especially in biological systems, the term “about” means within one log unit (i.e., one order of magnitude), preferably within a factor of two of a given value. Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups or component groups in any definition of a variable herein includes definitions of that variable as any single group or combination of listed groups. The recitation of an embodiment for a variable or aspect herein includes that embodiment as any single embodiment or in combination with any other embodiments or portions thereof as described in the disclosure.

Any compositions or methods provided herein can be combined with one or more of any of the other compositions and methods provided herein.

### BRIEF DESCRIPTION OF THE DRAWINGS

FIG. 1 presents a graphic of interneuronal cell types, namely, VIP, PV-Basket, PV Chandelier, LAMP-5, and SST, and enhancer accessibility patterns as determined by an enhancer sequence identification method, (also termed PIASO (“Precise Integrative Analysis of Single-cell Omics”)) herein, that identified enhancer element sequences that target certain neuronal and interneuronal cell types, subtypes and populations. The method enabled the identification of SST, PV, VIP subtype-specific enhancer sequences, as well as Lamp5-expressing cell subtype-specific enhancer sequences, based on analysis of scATAC-seq data. By way of particular example, the results of such analysis are illustrated in the genomic track plot showing a peak delineating the location of an SST-neuronal cell subtype specific enhancer sequence. The neuronal cell populations that are targeted by the enhancer elements described herein include classes of inhibitory GABA-ergic neurons, such as “PV”: parvalbumin expressing interneurons (e.g., PV-basket or PV-chandelier type PV interneurons); “SST”: Somatostatin expressing interneurons; and “VIP”: Vaso-Active Intestinal Peptide expressing interneurons.

FIG. 2 presents a graphic depiction of neuronal and interneuronal cell types and modalities associated with RNA and ATAC data that are integrated in the performance of the technique used to identify enhancer sequences (see FIG. 1). FIG. 2 demonstrates that the technique accurately integrated scRNA-seq data and scATAC-seq data collected from adult mouse cortex.

FIG. 3 presents peaks in genomic track plots showing that an isolated enhancer sequence, which was identified as targeting VIP interneurons in mice, also showed specific accessibility in human VIP interneurons in mouse and human cortical tissues.

FIG. 4 presents a schematic illustration of the experimental strategy used to test the specificity of isolated and cloned enhancers sequences identified according to the disclosure in targeting certain neuronal and interneuronal cell types, subtypes and populations thereof. The isolated enhancer elements were cloned into recombinant adeno-associated virus (rAAV) vector constructs to drive the expression of the dTomato reporter gene contained in the vector construct. The constructs packaged into AAVs were tested in mouse and non-human primates for their specificity in targeting neuronal and interneuronal cell types, subtypes and populations.

FIGS. 5A-5H present confocal microscope images of immunohistochemical analyses related to the targeting of specific neuronal cell populations by rAAV vectors harboring a reporter gene (d-tomato, “dTom”) and enhancer element sequences as described herein. The images shown in the figure demonstrate the specificity for interneuronal cell types of rAAV constructs containing the enhancer elements described herein and a reporter gene (dTom), “rAAV-Enhancer-dTomato vector,” following injection into mice. In brief, mice were systemically injected with 2×10¹¹ viral particles per adult mouse (FIGS. 5A-5G) or were injected with 5×10¹⁰ viral particles per mouse by intracerebroventricular injection at postnatal day 1 (FIG. 5H). The rAAV-Enhancer-dTomato vector expressed the reporter dTomato under the control of the enhancer sequence regulatory element. Brain sections of injected mice were analyzed 3 weeks post-injection by immunohistochemistry (IHC) for expression of both the reporter and the neuronal cell marker.

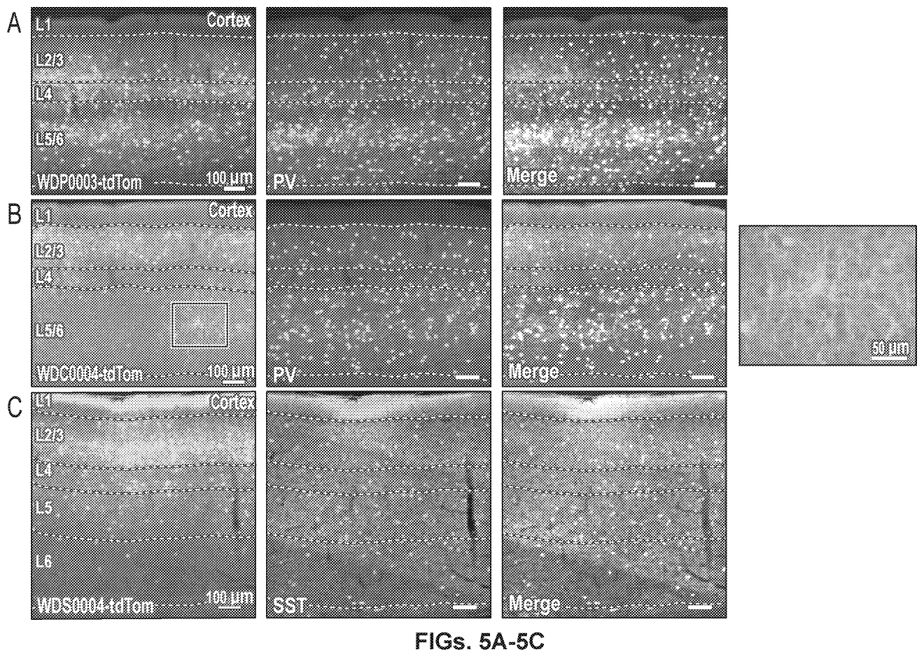

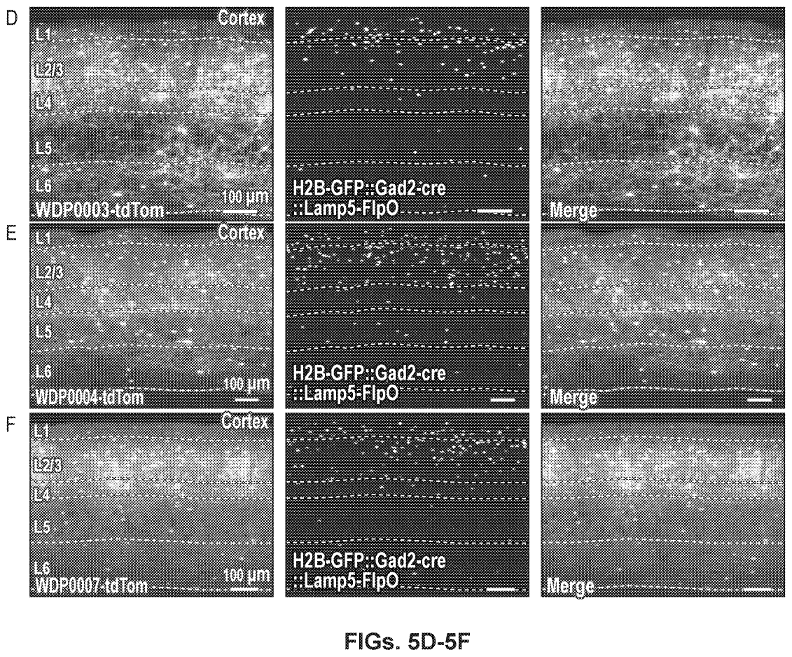

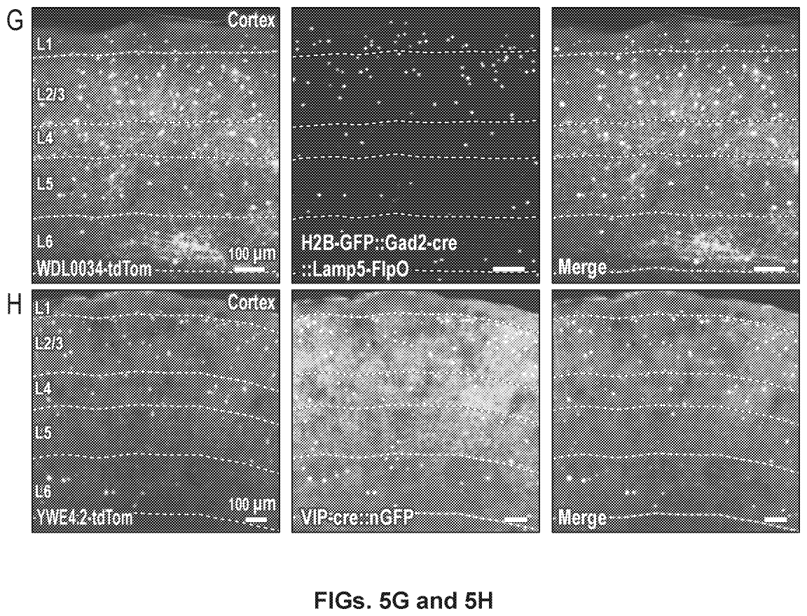

Detection of specific PV, VIP, SST, or Lamp5-expressing neuronal cells transduced by the vector is visualized. Shown are the results of the specific expression of the dTom reporter in interneuron cells in the brain using the enhancer element sequences identified and described herein. The images in row “A” (FIG. 5A) result from specific expression of the dTom reporter in PV-expressing basket cells in mouse brain using the ME1 (“WDP0003”), SEQ ID NO: 1, enhancer element cloned into an rAAV construct. The images in row “B” (FIG. 5B) result from specific expression of the dTom reporter in PV-expressing chandelier cells in mouse brain using the ME2 (“WDC0004”), SEQ ID NO: 2, enhancer element cloned into an rAAV construct (an enlarged view of the area within the box on the leftmost image of “B” is shown in the smaller image at the far right to show the unique morphology of PV-expressing chandelier cells). The images in row “C” (FIG. 5C) result from specific expression of the dTom reporter in SST-expressing cells in mouse brain using the ME3 (“WDS0004”), SEQ ID NO: 3, enhancer element cloned into an rAAV construct. The images in row “D” (FIG. 5D) result from specific expression of the dTom reporter in Lamp5-expressing interneuron cells in mouse brain using the ME4 (“WDL0003”), SEQ ID NO: 4, enhancer element cloned into an rAAV construct. The images in row “E” (FIG. 5E) result from specific expression of the dTom reporter in Lamp5-expressing interneuron cells in mouse brain using the ME5 (“WDL0004”), SEQ ID NO: 5, enhancer element cloned into an rAAV construct. The images in row “F” (FIG. 5F) result from specific expression of the dTom reporter in Lamp5-expressing interneuron cells in mouse brain using the ME6 (“WDL0007”), SEQ ID NO: 6, enhancer element cloned into an rAAV construct. The images in row “G” (FIG. 5G) result from specific expression of the dTom reporter in Lamp5-expressing interneuron cells in mouse brain using the ME7 (“WDL0034”), SEQ ID NO: 7, enhancer element cloned into an rAAV construct. The images in row “H” (FIG. 5H) result from specific expression of the dTom reporter in vasoactive intestinal peptide (VIP)-expressing interneuron cells in mouse brain using the ME8 (“YWE4.2”), SEQ ID NO: 8, enhancer element cloned into an rAAV construct. The images illustrate the specificity of expression for the target neuronal cell populations as shown by immunohistochemistry for the viral reporter and the indicated marker gene for the enhancers described herein having specificity above about 75%. All images show staining in the S1 region of the brain neocortex. The merged images reflect staining of both dTomato and the neuronal cell type. In FIGS. 5A-5H, the leftmost images show interneuroanl cells expressing the reporter gene (dTomato) (labeled in magenta). The middle images show genetically-labeled interneuron subtypes (labeled in green). The righmost images reflect merged images in which dTomato-positive cells that co-localize with cell type-specific markers are shown in white areas.

### DETAILED DESCRIPTION OF THE EMBODIMENTS

Described and provided herein are new enhancer element polynucleotide sequences that target certain cell types, subtypes, and/or populations. In particular, the newly provided enhancer sequences target certain neuronal cell types, subtypes, and/or populations. In an embodiment, the enhancer sequences are mouse enhancer polynucleotide sequences as set forth in SEQ ID NOs: 1-8. In an embodiment, the enhancer sequences are human ortholog enhancer sequences as set forth in SEQ ID NOs: 9-16, which are the human counterparts of the mouse enhancer sequences of SEQ ID NOs: 1-8, respectively.

As described herein, eight (8) mouse enhancer polynucleotide sequences (SEQ ID NOs: 1-8) were identified as being capable of targeting different neuronal cell types or subtypes and/or neuronal cell populations. Also described are the enhancer polynucleotide sequences that are the human counterparts of the mouse enhancer sequences. The human counterpart (ortholog) enhancer sequences were also identified and are provided in SEQ ID NOs: 9-16, respectively, herein. In an embodiment, the enhancer sequences that target certain neuronal cell types or subtypes can restrict the expression of a gene (e.g., a transgene) to the target neuronal cell type, subtype, or population. In an embodiment, the enhancer sequences that target certain neuronal cell types or subtypes can regulate or modify the expression of a gene the expression of a gene (e.g., a transgene) that is targeted to a neuronal cell, subtype, or population.

In an aspect, the isolated enhancer sequences as described herein were identified using a method as described herein, which relies on combining chromatin accessibility data with cross-species conservation to identify putative enhancer elements in the vicinity of genes enriched in the target neuronal population. In embodiments, the application of the method led to the identification of five candidate enhancer sequences for specific neuronal populations, namely, classes of inhibitory GABA-ergic neurons (i.e. parvalbumin-expressing interneurons (PV interneurons); Somatostatin expressing interneurons (SST interneurons); and Vaso-active Intestinal Peptide expressing interneurons (VIP interneurons). Table 1 herein presents the designations of mouse and human enhancer sequences, their genome coordinate and start/stop position, their target neuronal cell types, and SEQ ID NOs.

The isolated enhancer sequences SEQ ID NOs: 1-8 and human orthologs thereof (SEQ ID NOs: 9-16) as described herein can be used to restrict or regulate therapeutic interventions, such as, without limitation, gene replacement, gene modulation, gene editing, modulation of cellular activity, to distinct neuronal and interneuronal populations in the brain cortex and the central nervous system (CNS). The enhancer sequences identified and described herein have the potential to substantially improve current or future therapeutic approaches for the treatment of diseases, disorders, and pathologies of the CNS and the brain. The enhancer elements as identified and described herein may be employed across species and/or in cells of different species. By way of example, the mouse and human enhancer element sequences provided herein can be used to target and restrict the expression of genes, e.g., transgenes, in human neuron and interneuron cell types and can be utilized in other mammalian subjects.

In embodiments, an enhancer sequence (e.g., of any one of SEQ ID NOs: 1-8 or SEQ ID NOs: 9-16) was individually cloned into an AAV (e.g., rAAV) viral vector, which was used to produce AAV particles. In an embodiment, the rAAV viral vector also contained a gene of interest, e.g., a reporter gene (e.g., a transgene) to allow the identification of cell types and subtypes in which the gene of interest (reporter gene) was expressed. When administered to (e.g., injected into) mice, e.g., adult mice, the virus vector was capable of driving expression of the gene harbored in the vector exclusively in neuronal or interneuronal populations targeted for expression of the gene by the enhancer sequence.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME1,” located on chromosome 1, genome coordinate (mm10) at start/stop positions 61587715-61588378, or a human ortholog thereof. Enhancer sequence ME1 is also termed “WDP0003”. In an embodiment, the ME1 enhancer sequence targets parvalbumin (PV)-expressing basket cells. The ME1 sequence is as follows:

(SEQ ID NO: 1)
agaccgctgttgcagtatttcagggaacaagatcagaatggtatagccta
cctgaaaaataggcaatcctttcaaaattaaatgcactttctcttgggta
ggagacaagataatctatgataagggaagagagatgtctccagcttttat
tttttttcttgtaatttgcatttaagccctggtaacagtttaatatgaag
gtgactatggcagctccatttattcaatctatcaatactctgttgtcaca
ggaggagccatgaggacttgagtgcagaggaggaatccccacaaagcagt
cttcactcttatcatagcttccatgtgcccttcttattcagcagctctca
ttaaaggaacactatctttagcacagggattctacttgacattgacaagg
acaggctcagtagcaaatgggccaaaactatgaataggcgattcacagca
gagtaaaatccaatggcagcagacataaaaagaagctcaaaatgacaaac
aagtcagacaaacacaaattcaagtaacaataaaaaatggagcactggtt
tgactcgtacacaagcaaacagacacaaacaaaacaaaagagtcattaca
cctggggctaaagacttctcttatgtattgcaaagtggaaataagacctc
caggcaaaatgtga.

In an embodiment, a human neuronal cell enhancer sequence, which is the human ortholog (counterpart) of the above-described ME1, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE1,” i.e., human counterpart genome coordinate (hg38): chromosome 2 at start/stop positions 204505895-204506542. The huE1 sequence is as follows:

(SEQ ID NO: 9)
aggcaagtgttactatcttccaaggactaagatcagggtgatgtgaactg
aggaataggcattcctcttgaaattaaagtgtgtacattttcctaggtgc
tattatacaagaggatctattgagtagagaagggaaaatgccataatatt
tatttttgtatagtttttatttaagctctactgagaatttagtgtaaaaa
tgaccatggccacatcatgtggtcagtatgccaagatactggtgtcacgg
gattcgtgaggaattcctaatgaagagtaagaattccacaatgtagtcat
cattttattgcggttcgcatgtgccctgcttattcattagctctcacaaa
aaggacaatacccataatgcacaaagtctccttacaactaacaaggaaca
ggcccaataacaaaggtgccaaagatatgaacaggcaactcccagaagag
taaaatccaaatgctgacagacatataaaaagtagctcaaaattacaaac
aagtcagagaaatgcaaattaaagtaacaatgagaaagtgtgcattggtt
tgacaaaaaaatatgaagagccattataccttaggcaaaagagtattcct
gtgcattgcaagtagaaatgagaactgttacaggtaagatgcaggcaa.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME2,” located on chromosome 13, genome coordinate (mm10), at start/stop positions 25392743-25393267, or a human ortholog thereof. Enhancer sequence ME2 is also termed “WDC0004.” In an embodiment, the ME2 enhancer sequence targets parvalbumin (PV)-expressing chandelier cells. The ME2 sequence is as follows:

(SEQ ID NO: 2)
ggtgtatttcctgtgtgggctaatgtagctaactttgtttggactccagg
tttctgaggcagtttcaaattcttgatttatgggttcatttttggatcat
ggcagcctccctgctgaaacccccatagtttcccttcaataaggaaggct
ggatgagatcacagctgaggaatcctggtggtgtgtactgtgttatttct
ctcctagtgctttgcctacacccttccatcatgcttacatttggacctga
aaataattggttgaagctgctcctcacttttagccttttatccgagtttg
acagtacatcaccccaaataacacggactataaatggcttgaacctaatg
agtgctctttgtggacaagccgtccattctccttttcttgagagggaaat
taccggctcctaagcttctatttccagttctatagaatgtgggcgctaat
ttgaaagcagcctcataaaatggaagcaaacaggctaattagaaatattt
atgggcctgttagtgaatatatgcc.

In an embodiment, a human neuronal cell enhancer sequence, which is the human ortholog (counterpart) of the above-described ME2, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE2,” i.e., human counterpart genome coordinate (hg38): chromosome 6 at start/stop positions 24013075-24013559. The huE2 sequence is as follows:

(SEQ ID NO: 10)
gtttgcttccattttatgagactgctttcaaattagcacccacattctat
agaaatggaaagagaatcctggaagattgtaatttccctctcaagaaagg
aagaaggacaacagatcacatgtccataaagagtacttattaggttcaag
ccatttatagtacatgttattcggcacaacatacttgtcaaatttggata
aaaggctaaaagcgagtagcagttccaatcaattattttcaggtcctcaa
gcaaacatgatagaagagtgtaggcaaagcatgagaagagaaatagcatg
gtgcccaacaccatgatttttcaggccatgaccttggctggccttccgta
ttgaagaaaaaaaacaaagaagctctaccgggaccagagaatggttccag
aaatcaaggattcaaaagtgcatcatagcctctaaagttaggcaaagtta
acattagctatgttagaccacagagaaaatatgcc.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME3,” located on chromosome 10, genome coordinate (mm10), at start/stop positions 72204954-72205598, or a human ortholog thereof. Enhancer sequence ME3 is also termed “WDS0004.” In an embodiment, the ME3 enhancer sequence targets somatostatin (SST) neuronal cells. The ME3 sequence is as follows:

(SEQ ID NO: 3)
tttcagcataagcaagatggaatggacaatctcttatttcatcctaagtt
ttacaggaggtccttgttttatgtaaatttccaaccatttcaaaagctct
ttatgtgtgtattttttttttaaccatttttgacagtttcaactctttct
tctcccatgatttctttttgctctcaactgttatgggagagacaccaggt
tctgtatgcatttctgctttgggttgaaaattttatgcacttcaaatcaa
gcttcattcacatagctaaattcactgaggatacttcttccctttgaaaa
tttgacaagattcttaacaacccttccccgttctcctcatatttgttgag
tttcctagtgcatgtccttctccctgcccccagaataactagcaggtgtg
acacaggcatgcattttgcattttccaagtctgtgatgctaactttaggc
ctagaaacattagcagatttcctctttttatcatgcatacatttgctgct
tctgttatccaatctagtcactttaaagaattttcttatttcttttggga
cctgtgcaatctgttgcaagctagaaatgtccatggctagaccaataaat
tcactagataaatttacctgtggccacattatttaaaagtctgtg.

In an embodiment, a human neuronal cell enhancer sequence, which is the human ortholog of the above-described ME3, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE3,” i.e., human counterpart genome coordinate (hg38): chromosome 10 at start/stop positions 57176962-57177517. The huE3 sequence is as follows:

(SEQ ID NO: 11)
aacaatgtgacaaatgggaaatgtgcctaatgaattcattggtctagcta
aggaaatttccagacagaatgtagaaactgccagctgacaacaaattgaa
gataaatttctaacagtcttaaaagaaatgaacaaaattcttagagtgac
agtttgtaagcgaataatgaaagcaacaaagaaccctaggatgaaaaaga
taaactctgatgaaatatctgggcttaaagatatcatcagaggacttgaa
attacaaaatacatgctagtgtaaaatctttatgaaaatattacaaaatt
ttataaagaattttgtcatatttttaaagggaaaaggcatcccatatgaa
gctactttaaaaatggtgatgttcagtttaaaatgcataaatgttttcaa
ccaaaagcagagagagatagtgaaatgaatgactcttttgcagttgatta
tggcagcatgaaaattatgagaggagttagagttgaaactgtcagatgta
gttaaaatatgcacatacaggttacgaaattgttaaacgttttcataaaa
taagga.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME4,” located on chromosome 13, genome coordinate (mm10), at start/stop positions 96264516-96265144, or a human ortholog thereof. Enhancer sequence ME4 is also termed “WDL0003.” In an embodiment, the ME4 enhancer sequence targets Lamp5-expressing interneurons. The ME4 sequence is as follows:

(SEQ ID NO: 4)
aaaggatcttggccctgtacgcctaagcatcccttcggtttggtagcaat
ggatgattgtatgcaccattctggttcacatgagttttctttctttcttt
tgacagcatctgattatattagtgtgtattcatcaggaacaaaatggttc
catcattattcaagtgacttggattcagaatcactagacctatcgaatat
gcagatgcaaaggaaagttggtctattcagcctatcccgcaacaagccaa
gaaaaccctggcaccaatcaggaacaacaaaaatggaagaaagtatgtgc
ttaataatcggtatcattctctgaagtaaataaaagtgctcagattagtg
gcaaagctcattggttcttttaatatcaattgtaatgccatgaatttttc
ccttagctactgtttcttattgaaatgaactgaaataactctaaaagaaa
catctattatatgactgcaaatgttggagcagatataaatctgaataggc
ttatgagtagagtttctatctttgccactggcttttaattattttcctaa
atcacatataactttgatgaaagtgaatattgctattttctgtcagccat
ggttgaatgacatcaacacaggagccatt.

In an embodiment, a human neuronal cell enhancer sequence, which is the human ortholog of the above-described ME4, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE4,” i.e., human counterpart genome coordinate (hg38): chromosome 5 at start/stop positions 75926043-75926768. The huE4 sequence is as follows:

(SEQ ID NO: 12)
aatggcttctgttaagatctcacttaactgcggttgatagaaaatggcaa
tattcaatcaaagttatatataaattaggaaaagaattagaagccggggt
agcttgacacagatataaattctcactgagcttacccaaactctcatctg
ctccaaggcttacccaaactctcatctgcttacccaaactctcatctttg
agcttacccaaactctcatctgctccaagctgtatagaagtttctttcag
agttatttcagttcatttcaatgaaaaaattatcaaatataataaaacat
ttttggaaagacaggagggatttgggacattacaattggtattaaaagaa
ctaatgagctttgctactgataaagtatttcttgcattcttttttggttc
aaagagtgatacccaattattaattcttaagcacataaatcttttccatt
tttgtttttcctgctcggtgcctgaattttcttggctactactgaaatgt
gttgaataggctgattctactttttatctgcatattctatggatctagtg
gctctgaatccaagccactataataatgatggaaacaaaatatttctgat
gaacatataaccagatattgctgacaaatagaaaaaagaacaggctgatg
ggggccagactgtgcatatgatctgtattcctgccaaactgcatgtatgc
acagccacacaaagacaaaatccttt.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME5,” located on chromosome 18, genome coordinate (mm10), at start/stop positions 3795544-3796327, or a human ortholog thereof. Enhancer sequence ME5 is also termed “WDL0004.” In an embodiment, the ME5 enhancer sequence targets Lamp5-expressing interneurons. The ME5 sequence is as follows:

(SEQ ID NO: 5)
gctgtaagatgcccgagaggcagaatctgaagtcatcgtctcttctctct
tttttgttgttttcttgagatagagtcttgctctacagctcaggatagcc
ttaaacctgggatatcctcctgcttcctccttctgaatgctgagatttaa
gggtgcatctaccacagcagtctttgtctttagaaaacttctgaacatat
ttgaaaagtagaaacatggaaaatactttgtatctgtgcttccaaagaaa
gatctatatttgtgttctgtgctctgcttgccatattggccctgtgcatt
gatgaacagtgaagttaccttgttaagaagcatggaagggtcagaactag
ccactgtccctgatggccttctgctatggtaggaagaggtttgtttccaa
gggccgatcttatttttatgactgaattgtgtgacagcttgaagccctaa
atcaacacttctccttcattgggaatgagaaagaagccaggcagggaaga
gaatggcagatgaaactgggattattgtcagatggaccagccctggtaaa
cagtgtgtttctgacacacccagcacttcttctcagatctagctggatgc
cagactgtgcctgccagagacatgggctataattttgttgtttgaagcag
aaaaatgtgaagatatttaaaacttagttactcattccaaaatcagatgg
atccccaaataactgcctaaagaacagagtgtgaagagcaacctgaaaga
catgggaaagttctagccaaggtgaggtacaggt.

In an embodiment, a human neuronal cell enhancer sequence, which is the human ortholog of the above-described ME5, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE5,” i.e., human counterpart genome coordinate (hg38): chromosome 10 at start/stop positions 28905799-28906520. The huE5 sequence is as follows:

(SEQ ID NO: 13)
cttgtgagttgtagatgcccttaaatgccttctctatgaaaggacacagt
ccatacccatcagcatttttaattaggaaaactcccagaaaattatggta
tggacaaaagttgaggctttaaaaaaaaattcttggaaacacggaaaata
tttcacatctgaacttcagaagaaagatctattttcatattctggttttg
gctcaccagactggtcctgcgctttgataaatggcaaagttacctcgtta
atgagcactgaacagatccggccactgtccctgcttgggctctgtgctgg
gcaggaagcggttttctcccccgaggccagatctcattttcatgactgaa
tcttgtgagatcttgaagcactaaatcaataactcttattcagtcagccc
tgaaaggaaggcagttatcaaaaggaacgcaggcaaagcaggtgatagca
gatgaaactcggcgattattttcaaaagaaccagcaggctctgaagtctg
cggatgcttctctggcacaccccaaatcttcccctcacatccagtttggt
gccaagctgtgcctgtcgtcagtatagcgtgtacaatttagttgcttgaa
gcagaaaaaatgtgaaagtggccaatcttatgtcttacttacctagtcaa
aatgcatatggaaatgcaaagtgcttatgtctatattaaaaagaaaaaac
gcatccagcacagaaagacatg.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME6,” located on chromosome 2, genome coordinate (mm10), at start/stop positions 76120872-76121647, or a human ortholog thereof. Enhancer sequence ME6 is also termed “WDL0007.” In an embodiment, the ME6 enhancer sequence targets Lamp5-expressing interneurons. The ME6 sequence is as follows:

(SEQ ID NO: 6)
gttatgtttcagaggctccccaaccctgctcacctgaactcatccgttat
tcagtgggtcacaaactctagaacatcccagatagctcccgattcagctg
ctctgagtggggcctgagaatttacatgtttaacaagttcccagataaag
ctgataatgctgctagtttggggacatttctgtgaaaaccactggttaaa
gcctacccatgcattttccatgttaaaatggcccgtttgctctgccgaag
gctacaggttggatgtcagagtgtgagcagagaggaaaaagaccaatttg
actgcgtcttctcccaagccgcaatgtgatctgaataccttcgttcatga
aggctaaaaatgtctctttagttattttaataactatatttttgtgcctg
gatttcttttcatttcctaattccattgaaaatggagattaaaaaaagac
actatgaaatctgttccaatgaatatttctataaagcttttgacttccaa
aagcaatctgattagcatgctcttccaagccacacagtcatgtgagtggg
cccgctacatttctgagcagagaaaccagacttcatccctgagttgccat
cgatgtgtagcatgagttcattgtctccttttctgtgggaccagaggtag
catttcattttgtttatgtgaagtagttctttaatgaactatcagcaaga
attctcaagactggccatttctgtctagctggcaaacttggttttcctaa
ttatcaccgtttaggatgccagcatt.

In an embodiment, a human neuronal cell enhancer sequence, which is the human ortholog of the above-described ME6, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE6,” i.e., human counterpart genome coordinate (hg38): chromosome 2 at start/stop positions 177783273-177785047. The huE6 sequence is as follows:

(SEQ ID NO: 14)
gtcttagtgaggaatgctggggcctatggttagataacatttcattcaccttgttatacttc
agaagttctcatatgtgtcttaagttatttattagctcagtggttagattctagtcttgtcc
atgtttttgaggtggtttttttccccctggaatttggactgatttctaaattctttctgagc
aacaagtacccaaactaacattttcatatttgtcttccattttttctattaagctcattaaa
attactactaccttttttcctgaggccctgcaagctaaagcttattccttgcaatataggca
agaaaaatgagtcagattgccactgcattactcttctgtaactaaagatgctttaagtctaa
catctggatagattttgcccaaaattaacctttgtttttattctgtttccacaaaaacgcct
cttattgaaaatctgtttggctttgtatttcagacaaaagggtactggttttccagcctatt
cacttagatttcaaatggcattgatctcttcctataagcattgttaaactggggttaagcat
tttattaattatcactaggtgctatttcatttaaaaaattatttgttatattcaacaggtat
tcagatgggtaaagtttatatcttccaggctaccaaagttccagacaaacatgataattgac
tcaagggttccagcctgtgccactgcaggaaggactgagtccctataaatcattagataagg
tagtgagagatttctatgcctccagatcagatagagatgacagctcctgttcaggatgaagc
agttacagaagatgaaacctctgtccctctacaaccttttaggattaaggaagagtgcacag
tctctgaggagagatgaaaaaggaattcagcaggacttgtttcacaagatacagaccacaaa
gactgctgataaaacaggatgtggtaaagaagtctgccaaaacctgccaaaaccaagatggc
tatgaaaacaacctctggtcattctcagtgctcattatacactaattataatatattagcat
accaatggaaactccaccaacatcataacagtttacaattacaattacagttacaaatgccg
tggcattgtcctgaaactaccctatatgatctaaacgggagaggatccctcaattctgggaa
ctctcagctgctttcccagaaaactcatgaataatctactccttgtttagcacataatcaaa
aaataaccataagtatagtcagtcaagcagcccatgctgctatctgcctatggggtagccac
ccttttattcctttagttaataaagttgctttcactttactctgtcactttactgctggctt
tcactttactctatcactttatactttattgctggctcttgaattctttcctgcacaagcca
agaacccatgtgacctcccaagctgagttccaattttgaggtttgccctgtgacatctttgg
cttatgaaagcaatctgattaagatccagcttaaaattataatcatgtaagtcaactctact
acatagttttgagcacagaaatcagaattcatgctatgaattgccctaggcttgtagtctga
acttgttctctccctatttgcaggggtcagtggtaggatttaatctgtttatacggattaac
tgctgtatagattacctacagcaattcttaagacaggctgtttcctttcatttagtaaaact
ttgttttcttaatttgtcaatgcttagtgtgccaacaat.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME7,” located on chromosome 19, genome coordinate (mm10), at start/stop positions 43148967-43149558, or a human ortholog thereof. Enhancer sequence ME7 is also termed “WDL0034.” In an embodiment, the ME7 enhancer sequence targets Lamp5-expressing interneurons. The ME7 sequence is as follows:

(SEQ ID NO: 7)
ctcctctgctgggcagaaatgaggctgctacagtttgacagaagtttcccttgagttacatt
gtggtctgtctttattaaaagcaaaataaaagtcttatatatggggaactgccaactactcc
atattatgccaagttttcaatgtcttgctgttttgtgggagaagcagagaggaaggaggaag
ttatttgtaaattcgatacaaatagatctagccacgattatcttatcactgtcagtttaaat
atagagactcttttaactaaaaatcacaattagtctattaaaaatttgaaacatttcagaaa
gaaccatgagcatttttgatagagacaaaccgtaagtgcaaaagaaaccaagcttcaatagc
tctggctctttacactgaagtcagtaactacaacagggatgccagaattttactttatgcat
acatcatcatttaactcagcagctgatcatcagattcctagatggatggtggtggggcagga
aagcagtataatttatgtctgctatctgaggtaagtgggtgagagtgattctgaccagtatg
aaattcataggattatgtcaaaggacttgtcatg.

In an embodiment, a human neuronal cell enhancer sequence, which is the human ortholog of the above-described ME7, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE7,” i.e., human counterpart genome coordinate (hg38): chromosome 10 at start/stop positions 98916937-98917513. The huE7 sequence is as follows:

(SEQ ID NO: 15)
ctcctactgggaggaaatgaggctgctattggtctgactgaagttccccttaggctatatgt
ggtctgtctttattaaaagcaaaataaaggcctcacatggggacttggcaactattacaagg
gttgtgctgggtttttcgattcttgcttttttgttgggggtgtatgaaaggaatcaacagat
ccatccaagactattttatcattgtcaacttacatgcagaccaccctgacacccccactcac
tattagtctgttgaaaatgggaagaatttgagagggaaacactaacatttttaatagaaaca
attcttatagccaaataagaactaagctacaaaggttctaattatacattaggtattcaata
aacataatggtgattccagaattgtattttgtatgccatcatctaatttataagttggcatc
atcagattcaaacactgagtggaaaaaaactcagcaatataaattatataagcaatcatcac
gtgagtgggtggaaatgattctgaaacagataattctgaaatcaatcctgaaataggactat
ttgaaaggattaacttctg.

In an embodiment, a neuronal cell enhancer sequence as identified and described herein comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, at least 95% or greater, or at least 98% or greater sequence identity to the following mouse polynucleotide (DNA) sequence called “ME8,” located on chromosome X, genome coordinate (mm10), at start/stop positions 163532959-163533357 (on reverse strand), or a human ortholog thereof. Enhancer sequence ME8 is also termed “YWE4.2.” In an embodiment, the ME8 enhancer sequence targets vasoactive intestinal peptide (VIP)-expressing cells. The ME8 sequence is as follows:

(SEQ ID NO: 8)
tattttaaatcctcattggtcaggaatgtttacattcatagtgtgaagcagagcctgaaatg
ccaacttagtacttcagtgtgctgctaaattgactaagcgtgctgaacagtaatagcttggt
actagaagcccaatggcagggctcagtggccaaatgtttttattattacgaagtaataaggc
taataagcagtgttgtgtgatggaaggcaggcttacttctatttaaagatggaatgaacagc
tttgatggggacaagcaaagaacacaagaacattagctgtgttccgatattcagtgcacata
ggccagagcttggcaaacatttctgtaatggccagatgataaacatttgaggattggaagac
tatagggtctccaatgcaaccactaaa.

In an embodiment, a human neuronal cell enhancer sequence, which is the human ortholog of the above-described ME8, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huE8,” i.e., human counterpart genome coordinate (hg38): chromosome X at start/stop positions 16136397-16136750. The huE8 sequence is as follows:

(SEQ ID NO: 16)
gattttaaaccctcaatggccgggaactattatatccatggtgccaagcagagcctgagatg
ccaaattagtacttaaatatgctgttagactaagcatcctgagcgatgagtgcttagtactg
gaagaccaacgacaggggtcagtggcctattgtcttcattatcacaaattaataagtctaac
agacggtgctgtgtgagatggaaaatgagcttacctctgtttaaagaaggaaccaacagctt
tggtgtggacaggcagagaacaacagaaacggtaaaacttttggcaaacattttctgcagca
tattaggctttgtggaccatacagtctctattgcaactgctcaa.

Table 1 below summarizes the characteristics of the enhancer element sequences identified and described herein.

| | Genome coordinate: | Neuronal cell type | |
|---|---|---|---|
| Enhancer name | chromosome: position | specificity | SEQ ID NO: |
| ME1 (WDP0003) | (mm10): chr1: 61587715- | PV-expressing basket | SEQ ID NO: 1 |
| | 61588378 | cells | |
| HuE1 | (hg38): | PV-expressing basket | SEQ ID NO: 9 |
| (human counterpart of | chr2: 204505895- | cells | |
| ME1)_{—} | 204506542 | | |
| ME2 (WDC0004) | (mm10): chr13: 25392743- | PV-expressing | SEQ ID NO: 2 |
| | 25393267 | chandelier cells | |
| HuE2 | (hg38): chr6: 24013075- | PV-expressing | SEQ ID NO: 10 |
| (human counterpart of | 24013559 | chandelier cells | |
| ME2) | | | |
| ME3 (WDS0004) | (mm10): chr10: 72204954- | Somatostatin (SST)- | SEQ ID NO: 3 |
| | 72205598 | expressing cells | |
| HuE3 | (hg38): chr10: 57176962- | Somatostatin (SST)- | SEQ ID NO: 11 |
| (human counterpart of | 57177517 | expressing cells | |
| ME3) | | | |
| ME4 (WDL0003) | (mm10): chr13: 96264516- | Lamp5-expressing | SEQ ID NO: 4 |
| | 96265144 | interneurons | |
| HuE4 (human | (hg38): chr5: 75926043- | Lamp5-expressing | SEQ ID NO: 12 |
| counterpart of ME4) | 75926768 | interneurons | |
| ME5 (WDL0004) | (mm10): chr18: 3795544- | Lamp5-expressing | SEQ ID NO: 5 |
| | 3796327 | interneurons | |
| HuE5 (human | (hg38): chr10: 28905799- | Lamp5-expressing | SEQ ID NO: 13 |
| counterpart of ME5) | 28906520 | interneurons | |
| ME6 (WDL0007) | (mm10): chr2: 76120872- | Lamp5-expressing | SEQ ID NO: 6 |
| | 76121647 | interneurons | |
| HuE6 (human | (hg38): chr2: 177783273- | Lamp5-expressing | SEQ ID NO: 14 |
| counterpart of ME6) | 177785047 | interneurons | |
| ME7 (WDL0034) | (mm10): chr19: 43148967- | Lamp5-expressing | SEQ ID NO: 7 |
| | 43149558 | interneurons | |
| HuE7 (human | (hg38): chr10: 98916937- | Lamp5-expressing | SEQ ID NO: 15 |
| counterpart of ME7) | 98917513 | interneurons | |
| ME8 (YWe4.2) | (mm10): | vasoactive intestinal | SEQ ID NO: 8 |
| | chrX: 163532959- | peptide (VIP)- | |
| | 163533357 (on reverse | expressing cells | |
| | strand) | | |
| HuE8 (human | (hg38): chrX: 16136397- | vasoactive intestinal | SEQ ID NO: 16 |
| counterpart of ME8) | 16136750 | peptide (VIP)- | |
| | | expressing cells | |

Vectors, such as expression vectors, e.g., adeno-associated virus (AAV) vectors, recombinant adeno-associated virus (rAAV) vectors, containing an above-described, isolated and cloned enhancer sequence and/or an above-described, cloned enhancer sequence and another polynucleotide (e.g., a gene; a transgene; a therapeutic gene) can be used to drive the expression of the gene in a specific cell type (neuronal cell). In an embodiment, the use of such vectors can restore normal cellular function, e.g., by restoring expression of certain genes to the appropriate interneuron or neuron target cell populations. In this way, the cause of a disease or disorder, such as one that affects or is associated with neuronal or interneuronal cells is treated or ameliorated, for example, by restoring the excitation-inhibition balance in the neuronal or interneuronal cell or cell population.

Specific viral-based therapeutic products, compositions, methods and approaches for treating or ameliorating neurological, neurodevelopmental, neurodegenerative, neuropsychiatric, neurogenetic, or neuromuscular diseases, disorders, and pathologies are described herein. As described, virus vectors and vehicles for gene delivery are designed and produced to contain a specific enhancer sequence (enhancer) and a polynucleotide sequence of a gene of interest, e.g., a transgene or reporter gene, which is functionally expressed in certain interneuron or neuron cell populations following transduction of the interneuron or neuron cells by the virus vector or vehicle. In an embodiment, a virus vector or vehicle is provided which comprises the polynucleotide of a specific enhancer sequence (enhancer) and a polynucleotide encoding a given gene of interest, which is functionally expressed in certain interneuron or neuron cell populations following transduction of the interneuron or neuron cells by the virus vector or vehicle. In an embodiment, the enhancer harbored by the virus is capable of restricting the expression of the gene of interest to certain neuronal or interneuronal cells. In embodiments, the cells include inhibitory GABA-ergic neurons, namely, parvalbumin (PV)-expressing interneurons, Somatostatin (SST)-expressing interneurons; Vaso-active Intestinal Peptide (VIP)-expressing interneurons; or Lamp5-expressing interneurons, e.g., non-VIP/CGE-derived interneurons that express Lamp5. In embodiments, expression of the gene of interest is restricted to expression in cells that are deficient for that gene, or in cells that have a nonfunctional, mutated, abnormally expressed, or aberrantly expressed gene. In an embodiment, the expression of the gene of interest is specifically modulated in an interneuron cell or other neuronal cell. In other embodiments, the gene of interest is an effector gene, a therapeutic gene, or a reporter gene.

In an embodiment, the virus vector contains a specific enhancer sequence and a gene or polynucleotide of interest (e.g., a therapeutic gene or an effector gene as described above) associated with a neurological, neurodevelopmental, neurodegenerative, neuropsychiatric, neurogenetic, or neuromuscular disease, disorder, or condition, and the enhancer sequence is capable of targeting and/or restricting the expression of the gene or polynucleotide to an interneuron cell population that has loss-of-function for the gene, is deficient for the gene, or that expresses a mutant, variant, aberrant, or defective form of the gene associated with the neurological, neurodevelopmental, neurodegenerative, neuropsychiatric, neurogenetic, or neuromuscular disease, disorder, and pathology. In embodiments, the enhancer sequence inserted in the virus vector polynucleotide is one or more of ME1-ME8 (SEQ ID NOs: 1-8) or hE1-hE8 (SEQ ID NOs: 9-16) as described herein. In certain embodiments, the expression of the gene or polynucleotide of interest (e.g., reporter gene, therapeutic gene, or effector gene) in interneurons may be determined by the detection of markers that are specific for interneuron cells, e.g., without limitation, GABA GAD67, or PV interneuron cell markers. In an embodiment, the virus vector or vehicle is an adeno-associated virus (AAV) or a recombinant AAV (rAAV). The terms “AAV” and “rAAV” are used interchangeably herein.

A gene (or genes) or polynucleotide of interest (such as a reporter gene, a therapeutic gene, or an effector gene), also termed a “transgene” herein, is contained in the rAAV vector or vehicle as described herein and is expressed and functional in a certain cell types or populations as described herein, especially by virtue of the enhancer sequence also contained in the rAAV vector, which targets, restricts (or regulates) the expression of the gene to defined neuronal cell populations, namely, distinct classes of inhibitory GABA-ergic neurons, i.e. parvalbumin PV)-expressing interneurons; Somatostatin (SST)-expressing interneurons; Vaso-active Intestinal Peptide (VIP)-expressing interneurons and/or non-VIP/CGE-derived interneurons or subtypes thereof. In some cases, the gene or polynucleotide of interest (e.g., a therapeutic gene) is a normal form of a gene or polynucleotide that is expressed in the cell type transduced by rAAV and whose encoded product functions to provide a normal or normally-functioning product in the cell, such as, for example, a cell in which there is a loss of function of the same gene as the gene of interest or transgene. In some cases, the gene or polynucleotide of interest may be a reporter gene, e.g., green fluorescent protein (GFP) or red fluorescent protein (RFP) that provides a detectable signal following transduction of a cell by the rAAV vector. In some cases, the gene or polynucleotide of interest may be a clustered regularly interspaced short palindromic repeats-Cas9 (CRISPR-Cas9) protein or variant thereof, a Zinc Finger Protein, a Transcription activator-like effector nuclease (TALEN), or an engineered form thereof. In some cases, the gene or polynucleotide of interest may comprise a sequence containing both a reporter gene and a gene or polynucleotide that encodes a product whose expression and activity provide for normal cell function. The latter type of gene or polynucleotide may be considered to be a therapeutic gene. In a particular embodiment, the rAAV vector contains a specific, isolated and cloned enhancer sequence as described herein, namely, any one of SEQ ID NOs: 1-16, and a gene or polynucleotide of interest, such as a therapeutic gene for expression in a particular neuronal cell type in which the function of a gene corresponding to the gene or polynucleotide of interest, or the function of another gene in the neuronal cell is lacking, aberrant, mutated, silent, or otherwise defective. In some embodiments, the function or expression of the other gene is associated with the function or expression of the gene or polynucleotide of interest contained in the rAAV vector.

The rAAV vectors and methods described herein are based, at least in part, on the discovery and demonstration that an enhancer element contained in a viral vector can restrict the expression of a gene or polynucleotide contained in the virus vector (or in another virus vector), such as a gene associated with a neurological, neurodevelopmental, neurodegenerative, neuropsychiatric, neurogenetic, or neuromuscular disease, disorder, or pathology, or a reporter gene, to interneuron cells (“interneurons”), such as GABA-ergic interneurons in the brain where the disease-associated gene is expressed and the encoded product of the gene of interest is expressed and functional. In an embodiment, such an expressed, functional gene and its encoded product offsets, replaces, compensates, or substitutes for, the abnormal, aberrant, or lack of function of a gene encoding a product (a protein, polypeptide, or peptide product) which is involved in the normal functioning of the neuronal cell type or population.

In an embodiment, a suitable viral vector, e.g., a lentiviral vector or, in particular, an adeno-associated virus (AAV) vector, or a recombinant adeno-associated virus (rAAV) vector, is used to target and restrict expression of a gene or polynucleotide of interest in GABA-ergic PV-expressing interneurons in a mammal, in which an isolated, cloned enhancer element as described herein, e.g., mouse ME1 of SEQ ID NO: 1 or its human ortholog, HuE1 of SEQ ID NO: 9, or mouse ME2 of SEQ ID NO: 2 or its human ortholog, HuE2 of SEQ ID NO: 10, is provided in cis. In another embodiment, a suitable viral vector, e.g., a lentiviral vector or, in particular, an adeno-associated virus (AAV) vector, or a recombinant adeno-associated virus (rAAV) vector, is used to target and restrict expression of a gene or polynucleotide of interest in GABA-ergic SST-expressing interneurons in a mammal, in which an isolated, cloned enhancer element as described herein, e.g., mouse ME3 of SEQ ID NO: 3 or its human ortholog, HuE3 of SEQ ID NO: 11, is provided in cis. In another embodiment, a suitable viral vector, e.g., a lentiviral vector or, in particular, an adeno-associated virus (AAV) vector, or a recombinant adeno-associated virus (rAAV) vector, is used to target and restrict expression of a gene or polynucleotide of interest in GABA-ergic VIP-expressing interneurons in a mammal, in which an isolated, cloned enhancer element as described herein, e.g., mouse ME8 of SEQ ID NO: 8 or its human ortholog, HuE8 of SEQ ID NO: 16, is provided in cis. In another embodiment, a suitable viral vector, e.g., a lentiviral vector or, in particular, an adeno-associated virus (AAV) vector, or a recombinant adeno-associated virus (rAAV) vector, is used to restrict expression of a gene or polynucleotide of interest in GABA-ergic SST-expressing interneurons in a mammal, in which any one of isolated, cloned enhancer elements as described herein, e.g., mouse ME4-ME7 of SEQ ID NOs: 4-7, respectively, or their human orthologs, HuE4-HuE7 of SEQ ID NOs: 12-15, respectively, is provided in cis. By way of nonlimiting example, the described enhancer elements having specificity for SST interneurons in the cortex can be useful and advantageous for clinical intervention with the goal of reducing seizures and associated neurological and neuropsychiatric disorders. Targeting specific neuronal populations has beneficial utility and relevance for neurological, neurodevelopmental, neurodegenerative, neuropsychiatric, neurogenetic, or neuromuscular disorders, including, but not limited to, Alzheimer's disease, Parkinson's disease, Dystonia, ALS, Down Syndrome and epilepsy.

In an embodiment, the viral vector or rAAV vector comprising the enhancer drives the expression of a copy of a gene of interest in a transduced GABA-ergic neuron or interneuron, e.g., for the treatment and therapy of neuronal disorders, such as seizures or epilepsy. In other embodiments, the vector or rAAV vector comprising the enhancer drives the expression of effectors like Gq-DREADD or PSAM for chemogenetic modulation of PV-interneuron activity for the treatment of various forms of seizures, epilepsy, including focal and pharmacologically intractable epilepsy and/or the symptoms thereof. In another embodiment, the vector or rAAV vector comprising the enhancer drives the expression of a gene encoding an effector product such as CRISPR/Cas, ZFP, or TALEN proteins, and the like, for gene editing or gene expression modulation in particular neuronal cell types.

In general, a viral vector or rAAV vector comprises a polynucleotide comprising an enhancer sequence selected from any one of SEQ ID NOs: 1-16 as described herein, and a gene or polynucleotide sequence of interest, e.g., a gene encoding a desired product for expression in a cell. In an embodiment, the polynucleotide comprises an enhancer sequence selected from any one of SEQ ID NOs: 1-8 or any one of SEQ ID NOs: 9-16 as described herein. In certain embodiments, methods are provided for therapeutic and prophylactic treatments for a neurological, neurodevelopmental, neurodegenerative, neuropsychiatric, neurogenetic, or neuromuscular disease, disorder, or pathology in an individual (e.g., a human patient) in need thereof. By virtue of the enhancers described herein and contained in the delivery vectors, particular neuronal cell types associated with various neurological conditions are targeted and at least one gene of interest is introduced therein. In embodiments, interneurons, which are implicated in various neurological disorders and diseases, can be specifically targeted and a gene of interest (e.g., a therapeutic and/or effector gene) introduced into the cell types implicated in the neurological disorders, conditions and diseases, thus providing treatment and therapies for the neurological disorders, conditions and diseases. As such, the ability to target such neurons by using the enhancer elements described herein provides an advantageous therapeutic benefit for treating a variety of neurological conditions, such as, without limitation, Alzheimer's disease, Parkinson's disease, epilepsy and seizures.

In a certain embodiment, a method is provided in which an individual or subject in need, e.g., a patient afflicted with a neurological, neurodevelopmental, neurodegenerative, neuropsychiatric, neurogenetic, or neuromuscular disease, disorder, or pathology is administered a viral vector, such as a recombinant adeno-associated virus (rAAV) vector comprising an enhancer sequence as described herein and a gene or polynucleotide sequence of interest encoding, for example, a therapeutic protein-encoding polynucleotide sequence; a (Gq-DREADD)-encoding polynucleotide sequence; a PSAM-encoding polynucleotide sequence; a clustered regularly interspaced short palindromic repeats-Cas9 (CRISPR-Cas9)-encoding polynucleotide sequence, or variant thereof; a Zinc Finger Protein-encoding polynucleotide sequence; or a TALEN-encoding polynucleotide sequence, (or an engineered form thereof), such that the transgene product is expressed in intended neuronal cell populations of the individual or subject as described herein. Thus, a method is provided for converting interneurons and neurons, for example, PV-expressing interneurons, in an individual or subject in need, that do not express a given protein or polypeptide or that express a protein or polypeptide having abnormal or harmful activity to interneurons and neurons that express a protein or polypeptide having normal, non-aberrant, and/or non-harmful function or activity. As such, the directed expression of the genes and encoded proteins in neuronal target cells and cell populations is linked to the presence of the enhancer element selected from any one of SEQ ID NOs: 1-16 as described herein that is also provided as a component of the rAAV vector genome. In certain embodiments, methods are provided for therapeutic and prophylactic treatments for a neurological, neurodevelopmental, neurodegenerative, neuropsychiatric, neurogenetic, or neuromuscular disease, disorder, or pathology in an individual (e.g., a human patient) in need thereof, involving administering to the individual or patient a viral vector, such as a recombinant adeno-associated virus (rAAV) vector. comprising an enhancer sequence as described herein, and a transgene polynucleotide sequence encoding a protein or polypeptide to achieve a therapeutic treatment or effect.

In an embodiment, a prophylactic or therapeutic treatment method is provided for prophylaxis and/or therapy for neurological, neurodevelopmental, neurodegenerative, neuropsychiatric, neurogenetic, or neuromuscular diseases, disorders, or pathologies, including but not limited to, Alzheimer's disease, Parkinson's disease, Dystonia, ALS and Down Syndrome, epilepsy, which comprises introducing into an individual or subject in need a viral vector or an rAAV vector which comprises an enhancer sequence selected from SEQ ID NOs: 1-16 as described herein, and a transgene polynucleotide sequence such that the severity of the neurological, neurodevelopmental, neurodegenerative, neuropsychiatric, neurogenetic, or neuromuscular diseases or disorders and/or the symptoms experienced by the individual or subject is reduced, treated or prevented.

In an embodiment, a prophylactic or therapeutic treatment method is provided for prophylaxis and/or therapy for reducing seizures and associated neuropsychiatric disorders, which comprises introducing into an individual or subject in need a viral vector or an rAAV vector which comprises an enhancer sequence selected from any one of SEQ ID NOs: 1-16 as described herein, and a gene or polynucleotide sequence of interest, such that the severity of the seizures or the disorder and/or the symptoms experienced by the individual or subject is reduced, treated or prevented following expression of the gene and gene product of interest in a cell. In an embodiment, the individual or subject in need is experiencing a seizure (e.g., an epileptic seizure) at the time of administering the vector. Following administration of the vector to the individual or subject, the severity of the seizures and/or the symptoms thereof are reduced, treated, or prevented.

In a certain embodiment, a prophylactic or therapeutic treatment method is provided for prophylaxis and/or therapy for a neurological, neurodevelopmental, neurodegenerative, neuropsychiatric, neurogenetic, or neuromuscular disease, disorder, or pathology, such as seizures or epilepsy, and/or the symptoms thereof, which comprises introducing into an individual a viral vector or an rAAV vector which comprises an enhancer sequence selected from SEQ ID NOs: 1-16 as described herein, and a sequence encoding an hM3Dq modified muscarinic receptor (Gq-DREADD)-encoding polynucleotide sequence, and subsequently administering to the individual an effective amount of an agonist of the Gq-DREADD such that the severity of the disease, disorder, or pathology, such as seizures, epilepsy, and/or the symptoms thereof is reduced, treated or prevented. In an embodiment, the individual or subject in need is experiencing a symptom of the neurological, neurodevelopmental, neurodegenerative, neuropsychiatric, neurogenetic, or neuromuscular disease, disorder, or pathology (e.g., seizure or epileptic seizure) at the time of administering the agonist of the Gq-DREADD receptor. Following administration of the agonist, the severity of the disease, disorder, or pathology (e.g., seizure) is reduced. In embodiments, Gq-DREADD receptor agonist is clozapine-N4-oxide (CNO), perlapine, salvinorin B, salvinorin A, or another suitable Gq-DREADD receptor agonist as known and used in the art.

In embodiments of the therapeutic and prophylactic methods described herein, the individual or subject is experiencing, or is at risk for developing, a partial seizure or a generalized seizure. In other embodiments the individual or subject has, is suspected of having, or has been diagnosed with epilepsy of any form, including, without limitation, pharmaco-resistant epilepsy. In accordance with the described methods, seizures, epilepsy, or related symptoms are inhibited, blocked, reduced, abated, or prevented.

In an embodiment, a composition comprising a viral vector or rAAV vector is administered to a subject in need thereof. In an embodiment, the administration of a composition comprising a vector (or the vector itself) comprising an enhancer element, e.g., mouse or human enhancer of any one of SEQ ID NOs: 1-16, as described herein, and a gene or polynucleotide sequence of interest facilitates conversion of neurons or interneurons (e.g., certain GABA-ergic interneurons) of an individual or subject that do not express the gene of interest into neuronal or interneuronal cells which do express the gene of interest, e.g., in the brain or CNS. In another embodiment, the administration of a composition comprising a vector (or the vector itself) comprising an enhancer element, e.g., mouse or human enhancers having a sequence of any one of SEQ ID NOs: 1-16, as described herein and a polynucleotide encoding Gq-DREADD receptor facilitates conversion of interneurons or PV-expressing interneurons of an individual or subject that do not express Gq-DREADD receptor into Gq-DREADD receptor-expressing interneurons or PV-expressing interneurons in the brain, thereby resulting in interneurons or PV-expressing interneurons that are responsive to a Gq-DREADD agonist. In another embodiment, the administration of a composition comprising a vector (or the vector itself) comprising an enhancer element, e.g., any one of SEQ ID NOs: 1-16, as described herein and a polynucleotide encoding a PSAM facilitates conversion of interneurons or PV-expressing interneurons of an individual or subject that do not express PSAM into PSAM-expressing interneurons or PV-expressing interneurons in the brain. In embodiments, the vectors, compositions and methods as described herein are used in the prophylactic or therapeutic treatment of a neurological, neurodevelopmental, neurodegenerative, neuropsychiatric, neurogenetic, or neuromuscular disease, disorder, or pathology, e.g., Alzheimer's disease, Parkinson's disease, Dystonia, ALS and Down Syndrome. In embodiments, the vectors, compositions and methods as described herein are used in the prophylactic or therapeutic treatment of partial and/or generalized seizures.

In an embodiment, the vectors, compositions and methods as described herein are used in the prophylactic or therapeutic treatment of a number of different neurological, neurodevelopmental, neurodegenerative, neuropsychiatric, neurogenetic, or neuromuscular diseases, disorders, conditions, or pathologies. In an embodiment, the enhancers described herein target interneurons. In an embodiment, the vectors, compositions and methods as described herein are used in the prophylactic or therapeutic treatment of various forms of epilepsy, including, without limitation, pharmaco-resistant epilepsy and/or may constitute a replacement of a pharmacological treatment. In embodiments, the vectors, compositions and methods as described herein are used in the prophylactic or therapeutic treatment of one or more seizure disorders, which include, but are not limited to, epilepsy, including, localization-related epilepsies, generalized epilepsies, epilepsies with both generalized and/or local seizures, and the like, seizures associated with Lennox-Gastaut syndrome, seizures as a complication of a disease or condition (such as seizures associated with encephalopathy, phenylketonuria, juvenile Gaucher's disease, Unvericht-Lundborg's progressive myoclonic epilepsy, stroke, head trauma, stress, hormonal changes, drug use or withdrawal, alcohol use or withdrawal, sleep deprivation, fever, infection, brain cancer, and the like, or chemically-induced seizure disorders.

In some embodiments, the administration of a viral vector or rAAV vector comprising an enhancer element as described herein and a gene of interest, e.g., an ion channel encoding gene; an enzymatically active gene, may occur at a time prior to the onset of the neurological, neurodevelopmental, neurodegenerative, neuropsychiatric, neurogenetic, or neuromuscular disease, disorder, or pathology, or symptom thereof, e.g., a seizure or epileptic seizure, for example, days, weeks, months, or years prior to administration. By way of example, those in the art have demonstrated that rAAV driven expression can last for at least six years in a non-human primate model (Rivera, V. M. et al., 2005, Blood, 105:1424-1430).

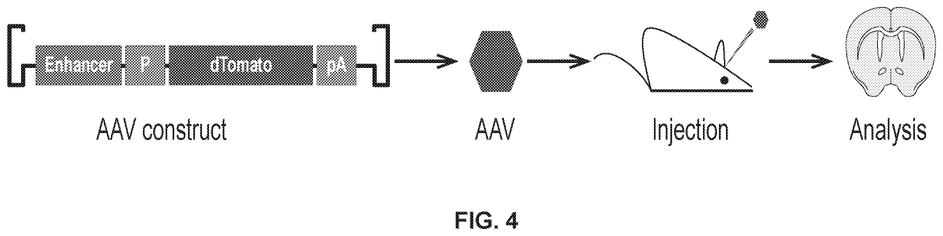

In an embodiment, the rAAV vector, which comprises an enhancer sequence as described for restricting expression of a gene of interest, e.g., a transgene, in particular neuronal and interneuronal populations and cell types, also comprises capsid proteins that enhance the targeting ability of the virus vector and allow the vector to specifically transduce interneuron cells, such as GABA-ergic interneuron cells, and/or specific subpopulations of GABA-ergic interneuron cells, particularly in the cerebral cortex of the brain. rAAV vectors that transduce GABA-ergic interneurons and rAAV vectors that comprise capsid proteins which increase the likelihood that the virus will specifically transduce GABA-ergic interneurons, in particular, the subpopulation of GABA-ergic interneurons that also expresses parvalbumin (PV), called PV-expressing interneurons, (also called PV-expressing cortical interneurons) are suitable for use in the compositions and methods described herein. By way of example, specific expression of a given gene or gene of interest, e.g., a reporter gene, a transgene, in PV-expressing interneurons in the brain may be achieved using the isolated and cloned enhancer sequences ME1 or ME2 as set forth in SEQ ID NO: 1 or 2, respectively, or the human orthologs thereof, huE1 or huE2, as set forth in SEQ ID NO: 9 or 10, respectively, in an rAAV vector (FIG. 4; FIGS. 5A, 5B). In other embodiments, an rAAV vector harbors the ME3 enhancer sequence of SEQ ID NO: 3 or its human counterpart huE3 enhancer sequence of SEQ ID NO: 11 and specifically transduces SST interneurons. (FIG. 5C); or the rAAV vector harbors any one of the ME4-ME7 enhancer sequences of SEQ ID NOs: 4-7, respectively, or the human counterpart huE4-huE7 enhancer sequences of SEQ ID NOs: 12-15, respectively, and specifically transduces Lamp5 interneurons in the brain. (FIGS. 5D-5G); or the rAAV vector harbors the ME8 enhancer sequence of SEQ ID NO: 8 or its human counterpart huE8 of SEQ ID NO: 11 and specifically transduces VIP interneurons. (FIG. 5H). In an embodiment of the foregoing, the rAAV vector containing the isolated, cloned enhancer sequence can comprise capsid proteins that enhance the targeting ability of the virus vector and allow the vector to specifically transduce specific interneuron types, such as PV-expressing interneurons, SST-expressing interneurons, Lamp5-expressing interneurons, or VIP-expressing interneurons.

Methods utilizing an AAV vector, which is designed and molecularly engineered to harbor an enhancer as described herein that restricts that expression of a given gene (e.g., a transgene) to certain interneuron cells, involve administering a therapeutically effective amount of the viral vector, a viral particle, a virus-like particle, or a pharmaceutical composition comprising the viral vector, particle, or virus-like particle to a subject in need, in particular, to transduce neurons or interneuron cells in the subject with the vector harboring the enhancer sequence and the gene of interest, express the gene in the neuron or interneuron cells and provide a functional response, e.g., the provision of a functional gene and/or gene product, or modulation of a gene or gene product, such as an increase in function of the gene or gene product, in neuron or interneuron cells of the subject following administration. By way of example, the functional expression of the transgene in the transduced neurons or interneuronal cells normalizes the excitability of neuronal or interneuron cell populations that are deficient in the gene, such as GABA-ergic interneurons, e.g., parvalbumin (PV)-expressing interneurons, Somatostatin (SST)-expressing interneurons; and Vaso-active Intestinal Peptide (VIP)-expressing interneurons; non-VIP/CGE-derived interneurons (Lamp5-expressing interneurons).

In an embodiment, the enhancer polynucleotide sequence that restricts, regulates, or modulates the expression of a gene of interest, (e.g., a transgene, a therapeutic gene) in an interneuron cell is about 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1050, 1100, 1150, 1200, 1250, 1300, 1350, 1400, 1450, 1500, 1550, 1600, 1650, 1700, 1650, 1800, 1850, 1900, 1950, 2000, 2050, or 2500 nucleotides (base pairs (bp)), or longer, e.g., greater than 2500 nucleotides (bp) in length, including the bp values at the beginning and end of the ranges, and all larger and smaller values in between these aforementioned bp lengths. The sequences of mouse enhancer polynucleotide sequences and their human ortholog sequences that target the different intended neuronal and interneuronal cell types described herein are provided in SEQ ID NOs: 1-16. In embodiments, the enhancer sequence for restricting, regulating, or modulating (e.g., enhancing) expression of a gene of interest or transgene (e.g., a therapeutic gene or effector gene) in a target interneuron or neuron cell and/or population may be derived from an intronic or intergenic sequence of a genomic polynucleotide, e.g., DNA or RNA.

### Types of Neurons and Interneurons

### GABA-Ergic Cortical Interneurons

GABA-ergic interneurons, which release the neurotransmitter gamma-aminobutyric acid (GABA) are inhibitory neurons of the central nervous system and are essential for regulating and maintaining neural circuitry and activity. (Kelsom, C. and Lu, W., 2013, Cell Biosci., 3:19). GABA-ergic interneurons of the mammalian cerebral cortex comprise several different cortical interneuron subtypes that may be categorized and classified by their expressed protein markers.

Interneurons play a key role in the wiring and neural circuitry of the developing nervous system of both invertebrate and vertebrate organisms. In general, an interneuron is a specialized type of neuron (nerve cell) whose primary role is to form a connection between other types of neurons. Interneurons, which are neither motor neurons nor sensory neurons, differ from projection neurons in that projection neurons send their signals to more distant locations, such as the brain or the spinal cord. Critically, interneurons function to modulate neural circuitry and circuit activity. A large majority of interneurons of the central nervous system are of the inhibitory type. In contrast to excitatory neurons, inhibitory cortical interneurons typically release the neurotransmitters gamma-aminobutyric acid (GABA) and glycine. Cortical interneurons are localized in the cerebral cortex, which is defined as a sheet of outer neural tissue that functions to cover the cerebrum and cerebellum structures in the brain. (Id.)

GABA-ergic interneurons include numerous interneuron subtypes that may be categorized by the surface markers they express. Four major cortical interneuron subtypes are parvalbumin (PV)-expressing interneurons, somatostatin (SST)-expressing interneurons (which constitute a heterogeneous population), and ionotropic serotonin receptor 5HT3a (5HT3aR)-expressing interneurons. These three subtypes together account for approximately 100% of the neocortical GABA-ergic interneuron population in mice. Although these interneurons home to their respective layers of the cerebral cortex, they are generated in various subpallial locations and they subsequently migrate to the cerebral cortex. GABA-ergic interneurons control information flow in the cortex by targeting specific domains of the principal neurons and thereby controlling specific spatiotemporal aspects of their activity. GABA-ergic interneurons are believed to play important roles in controlling the timing of pyramidal cell firing, synchronizing network activity, and the generation of cortical rhythms, as they respond to dynamic changes in excitation, increasing the dynamic range of cortical circuits, controlling sensory receptive fields and plasticity, and maintaining the excitatory and inhibitory balance necessary for the transfer of information while preventing runaway excitation. (B. Rudy et al., 2011, Dev. Neurobiol., 71 (1): 45-61).

Cortical circuit function is maintained by the balance between excitatory inputs and inhibitory inputs. A disruption of the balance of neural circuits is likely to contribute to the emergence of neurological, neurodevelopmental, neurodegenerative, neuropsychiatric, neurogenetic, or neuromuscular diseases, disorders, or pathologies, such as, without limitation, seizures, epilepsy, autism spectrum disorders, and intellectual disabilities.

### The Role of GABA-Ergic Cortical Interneurons

In the brain neocortex, neurons releasing the neurotransmitter GABA are, throughout most of the life of an animal, local inhibitory interneurons that are believed to play fundamental roles in shaping cortical circuits. In addition to releasing GABA, these cells are a source of neuropeptides that have important functions in modulating cortical function. Because of the large diversity of GABA-ergic interneurons that have arisen through evolution, the cerebral cortex can perform complex operations. Interneuron subtypes differ in morphology, intrinsic membrane properties, connectivity, and the efficacy and dynamics of input and output synapses, and these differences are associated with the expression of specific molecular markers. (B. Rudy et al., 2011, Dev. Neurobiol., 71 (1): 45-61).

GABA-ergic neurons play an inhibitory role and synaptically release the neurotransmitter GABA to regulate the firing rate of target neurons. Neurotransmitter release typically acts through postsynaptic GABAA ionotropic receptors in order to trigger a neuronal signaling pathway. Interneuron role/function is typically categorized into three components: (1) afferent input, (2) intrinsic properties of the interneuron, and (3) targets of the interneuron. In general, interneurons receive input from various sources, including pyramidal cells, as well as cells from other cortical and subcortical regions. (Kelsom, C. and Lu, W., 2013, Cell Biosci., 3:19). With regard to output, cortical interneurons engage in feed-forward and feedback inhibition. Regardless of the mode of output, the cortical interneuron network is further complicated by the fact that a single cortical interneuron is capable of making multiple connections with its excitatory neuronal target(s).

### Cortical Interneuron Subtypes

It is estimated that there are over 20 different subtypes of GABA-ergic interneurons in the cerebral cortex. The subtypes are also distinguished from each other based upon the calcium-binding proteins they express, which serve as markers. Based on studies performed in both mouse and rat brain tissue, the calcium-binding protein, parvalbumin (PV), and the neuropeptide somatostatin (SST), are key markers found to define the most predominant interneuron subtypes within the cerebral cortex. Of particular note, the PV-expressing interneuron population is independent from the SST-expressing population, in that expression of these markers does not overlap. In addition to PV- and SST-positive GABA-ergic interneurons, which together comprise approximately 70% of the total GABA-ergic cortical interneuron population, another subgroup of interneurons that express 5HT3aR were found to comprise approximately 30% of all interneurons. These three interneuron subpopulations account for nearly or equal to 100% of all GABA-ergic cortical interneurons, yet each of these populations, especially the 5HT3aR-expressing population, is heterogeneous and expresses other proteins or neuropeptides that contribute to their characterization. (Kelsom, C. and Lu, W., 2013, Cell Biosci., 3:19).

### Parvalbumin (PV)-Expressing Interneurons

PV-expressing interneuron represent approximately 40% of the GABA-ergic cortical interneuron population. This population of interneurons possesses a fast-spiking pattern, and fire sustained high-frequency trains of brief action potentials. These interneurons also possess the lowest input resistance and the fastest membrane time constant of all interneurons.

Two types of PV-interneurons comprise the PV interneuron group: basket cells and chandelier cells. Basket cells are interneurons that make synapses at the soma and proximal dendrite of target neurons, and usually have multipolar morphology. Several studies have shown that fast-spiking basket neurons are the dominant inhibitory system in the neocortex, where they mediate the fast inhibition of target neurons, among many other functions. Such fast-spiking basket neurons likely play a large role in regulating the delicate balance between excitatory and inhibitory inputs in the cerebral cortex. Unlike basket neurons, the chandelier cell subgroup of PV-expressing interneurons targets the axon initial segment of pyramidal neurons. Both basket cells and chandelier cells are fast-spiking, but they differ in electrophysiological properties. In contrast to other interneurons, chandelier cells may be excitatory rather than inhibitory due to their depolarizing effects on membrane potential. (Kelsom, C. and Lu, W., 2013, Cell Biosci., 3:19).

Another group of PV-expressing cells that is independent from chandelier and basket neurons in the neocortex, e.g., mouse neurocortex, are called multipolar bursting cells, which differ from chandelier and basket cells in both electrophysiology and connectivity. Multipolar bursting neurons possess synapses with pyramidal cells (or other multipolar bursting cells) that demonstrate a paired-pulse facilitation; in contrast, chandelier and basket cells are usually strongly depressing. (Kelsom, C. and Lu, W., 2013, Cell Biosci., 3:19).

### Somatostatin (SST)-Expressing Interneurons

SST-expressing interneurons constitute the second-largest interneuron group in the mouse neocortex and represent approximately 30% of the total cortical interneuron population. SST GABA-ergic interneurons represent a heterogeneous population of cortical interneurons. SST-positive interneurons are called Martinotti cells and possess ascending axons that arborize layer I of the cerebral cortex and establish synapses onto the dendritic tufts of pyramidal neurons. Martinotti cells are also found throughout cortical layers II-VI, but are most abundant in layer V. In contrast to PV-positive interneurons, excitatory inputs onto Martinotti cells are strongly facilitating. Additional subpopulations of SST-expressing cortical interneurons show differences in firing properties, expression of molecular markers and connectivity of different neurons within this population. (Kelsom, C. and Lu, W., 2013, Cell Biosci., 3:19).

### 5HT3aR-Expressing Interneurons

The third population of GABA-ergic cortical interneurons is designated as the 5HT3aR interneuron group, which accounts for approximately 30% of the GABA-ergic cortical interneuron population. Based on mouse studies, this population of GABA-ergic interneurons in the cortex express the 5HTa3 receptor, but do not express either PV or SST. 5HT3aR interneurons represent a heterogeneous population. Within the 5HT3aR interneuron group are several subsets of interneurons that also express other protein or neuropeptide markers, including vasoactive intestinal peptide (VIP). VIP-expressing interneurons are localized in cortical layers II and III. The VIP-expressing interneurons do not express PV or SST, but do express the 5HTa3 receptor, accounting for approximately 40% of the 5HT3aR population. VIP interneurons generally make synapses onto dendrites; some have been observed to target other interneurons. Compared with other cortical interneurons, VIP interneurons possess a very high input resistance and are among the most excitable of interneurons.

60% of cortical interneurons in the 5HT3aR-expressing population do not express VIP. Of this VIP-negative 5HT3aR group, nearly 80% express the interneuron marker reelin. In this latter category of cortical interneurons, the neurogliaform cell population, called spiderweb cells, express neuropeptide Y (NPY), and exhibit multiple dendrites radiating from a round soma. Neurogliaform interneurons can form synaptic connections with each other as well as with other interneuron types, in contrast to other types of interneurons that can only make synapses onto homologous neurons. Thus, neurogliaform cells play an important role in regulating neural circuitry and function by activating slow GABAA and GABAB receptors in order to provoke long-lasting inhibitory postsynaptic potentials onto pyramidal neurons and other interneurons.

### Lamp5-Expressing GABA-Ergic Cortical Interneurons

Lamp5 (Lysosome-associated membrane protein 5)-expressing GABA-ergic cortical interneurons, known as Lamp5 interneurons, are a specialized subtype of cortical interneurons located in the cerebral cortex. LAMP5 is a mammalian ortholog of the Caenorhabditis elegans protein, UNC-46, which functions as a sorting factor to localize the vesicular GABA transporter UNC-47 to synaptic vesicles of the organism. Lamp5 interneurons produce and release gamma aminobutyric acid (GABA), an inhibitory neurotransmitter. Within the cortical microcircuitry of the brain, the Lamp5 GABA-ergic cortical interneurons play crucial roles in modulating neuronal excitability and synaptic transmission. GABA release from these interneurons results in the inhibition of post-synaptic neurons, effectively controlling their activity rates and preventing unchecked excitatory responses, which aids in regulating the balance between excitation and inhibition. This balance is vital for maintaining normal brain functions; disruption of this balance often leads to neuropathological diseases, disorders, or conditions, such as epilepsy. Lamp5 GABA-ergic cortical interneurons are also involved in non-synaptic communication. Beyond the synaptic cleft, these interneurons are known to influence the activity of surrounding neuronal networks through volume transmission—a form of communication where GABA is released into the extracellular space affecting nearby neurons. Thus, Lamp5 GABA-ergic cortical interneurons can impact both local and broader functional connectivity within the brain, allowing them to significantly contribute to various complex cognitive processes. LAMP5 was found to be a regulator of hyperexcitation in mice and was critical for the survival of distinct interneuron populations in a mouse model of Alzheimer's disease (AD). In particular, synaptic LAMP5 was lost in AD brains, and LAMP5 interneurons degenerated in different AD mouse models. Genetic reduction of LAMP5 augmented functional deficits and neuronal network hypersynchronicity in both amyloid-β (Aβ)- and tau-driven AD mouse models. (Y. Deng et al., 2022, Acta Neuropathol., Vol. 144, No. 4, pages 637-650),

### Non VIP/Caudal Ganglionic Eminence (CGE) Neurons

The caudal ganglionic eminence (CGE), which is a fusion of the rostral medial and lateral ganglionic eminence, begins at the mid to caudal thalamus and is the second largest source of cortical interneurons, contributing approximately 30% of all cortical interneurons (G. Miyoshi et al., 2010, J. Neuroscience, 30:1582-1594). CGE-derived cells include GABA-ergic interneurons, spiny interneurons, mossy cells, pyramidal and granule neurons, and even oligodendrocyte and astrocyte glial cells. Recently, it has been shown that CGE-derived interneurons specifically express the serotonin receptor 5HT3a, while Nkx6.2 and CoupTF1/2 are widely, but not selectively, expressed within the CGE (R. Batista-Brito et al., 2013, In: Patterning and Cell Type Specification in the Developing CN-+a and PNS, Eds. J. L. R. Rubenstein and P. Rakic, Elsevier Inc., Academic Press; L. Lim et al., 2018, Neuron, Review, doi, org/10,1016/j.neuron.2018.10.009).

### Adeno-Associated Virus (AAV)

Recombinant AAV (rAAV) can drive long-term gene expression in vivo and therefore has become a popular gene delivery vector for research and gene therapy. To better leverage the use of rAAV, both in scientific and medical research and in medical and clinical therapeutics, the expression of a gene or transgene contained in a rAAV vector needs to be regulated in a tissue/cell-type specific- or context-dependent manner. To address this, efforts have been made to modify the AAV capsid. However, the extent of specificity capsid choice can offer is relatively limited. Enhancers, which are non-coding elements that can regulate gene expression and can control the activity or spatial-temporal pattern of gene expression, have been identified as described herein, that, when incorporated into rAAV vectors, can restrict the expression of a gene contained in the vector to particular neuronal cells, cell types and subtypes, and populations. The enhancer sequences described herein and contained in rAAV vectors advantageously allow for accessing and manipulating specific neuronal subtypes, in order to overcome the limitations of other approaches to achieve specific targeting of neuronal cell types and specific neuronal cell type-specific gene expression.

In particular, AAV is a small (25 nm), nonenveloped virus that contains a linear single-stranded DNA genome packaged into the viral capsid. It belongs to the family Parvoviridae and is of the genus Dependovirus, because productive infection by AAV occurs in the presence of either an adenovirus or herpesvirus helper virus. In the absence of helper virus, AAV (serotype 2) can establish latency after transduction into a cell by specific but rare integration into chromosome 19q13.4. Accordingly, AAV is the only mammalian DNA virus known to be capable of site-specific integration. (Daya, S. and Berns, K. I., 2008, Clin. Microbiol. Rev., 21 (4): 583-593).

There are two stages to the AAV life cycle after successful infection: a lytic stage and a lysogenic stage. In the presence of adenovirus or herpesvirus helper virus, the lytic stage persists. During this period, AAV undergoes productive infection characterized by genome replication, viral gene expression, and virion production. The adenoviral genes that provide helper functions for AAV gene expression include E1a, E1b, E2a, E4, and VA RNA. While adenovirus and herpesvirus provide different sets of genes for helper function, they both regulate cellular gene expression and provide a permissive intracellular milieu for a productive AAV infection. Herpesvirus aids in AAV gene expression by providing viral DNA polymerase and helicase as well as the early functions necessary for HSV transcription.

In the absence of adenovirus or herpesvirus, AAV replication is limited; viral gene expression is repressed; and the AAV genome can establish latency by integrating into a 4-kb region on chromosome 19 (q13.4), called AAVS1. The AAVS1 locus is near several muscle-specific genes, TNNT1 and TNNI3. The AAVS1 region itself is an upstream part of the gene MBS85 whose product has been shown to be involved in actin organization. Tissue culture experiments suggest that the AAVS1 locus is a safe integration site.

Recombinant AAV (rAAV) as a Vector for Gene Delivery and Therapeutic Treatment

AAVs are well suited for use as vectors and vehicles for gene transfer to the nervous system, as they enable gene expression and knockdown, gene editing, circuit modulation, in vivo imaging, disease model development, and the assessment of therapeutic candidates for the treatment of neurological, neurodevelopmental, neurodegenerative, neuropsychiatric, neurogenetic, or neuromuscular diseases, disorders, or pathologies. AAVs provide safe, long-term expression in the nervous system. Most of the foregoing applications rely on local AAV injections into the adult brain to bypass the blood-brain barrier (BBB) and to restrict transgene expression temporally and spatially.

AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to interneurons in brain tissue, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21 (4): 583-593). In addition, rAAV has been approved by the FDA for use as a vector in at least 38 protocols for several different human clinical trials. AAV's lack of pathogenicity, persistence and its many available serotypes have increased the potential of the virus as a delivery vehicle for a gene therapy application in accordance with the described compositions and methods.

Recombinant AAV (rAAV) vectors have been constructed that do not encode the replication (Rep) proteins and that lack the cis-active, 38 base pair integration efficiency element (IEE), which is required for frequent site-specific integration. The inverted terminal repeats (ITRs) are retained because they are the cis signals required for packaging. Thus, current recombinant AAV (rAAV) vectors persist primarily as extrachromosomal elements.

Recombinant AAV (rAAV) vectors for gene therapy have been based mostly on the AAV-2 serotype. AAV-2-based rAAV vectors can transduce muscle, liver, brain, retina, and lungs, requiring several weeks for optimal expression. The efficiency of rAAV transduction is dependent on the efficiency at each step of AAV infection, i.e., virus binding, entry, trafficking, nuclear entry, uncoating, and second-strand synthesis.

Several novel AAV vector technologies have been developed to either increase the genome capacity for AAV or enhance gene expression. Trans-splicing AAV vectors have been used to increase the capacity of the vector for harboring heterologous polynucleotides by taking advantage of AAV's ability to form head-to-tail concatemers via recombination in the ITRs. In this approach, the transgene cassette is split between two rAAV vectors containing adequately placed splice donor and acceptor sites. Transcription from recombined AAV molecules, followed by the correct splicing of the mRNA transcript, results in a functional gene product. While somewhat less efficient than rAAV vectors, trans-splicing AAV vectors permit delivery of therapeutic genes up to 9 kb in size and have been successfully used for gene expression in the retina, lung and muscle.

Polynucleotides encoding rAAVs as described herein comprise an enhancer polynucleotide sequence of any one of SEQ ID NOs: 1-16. Because of its nature as an enhancer, the orientation of the enhancer polynucleotide sequence, i.e., 5′-3′ or 3′-5′, is not material to its function. Accordingly, the enhancer sequences as described herein may be used in a reverse orientation and may be used as reverse-complementary sequences. By way of example, a “PV-specific enhancer” refers to the enhancer sequences described herein that target and restrict, regulate, or modify the expression of a gene (transgene) in PV-expressing cortical interneurons (PV-cINs) as described herein.

Moreover, the enhancer sequence need not be specifically spaced relative to other sequences, such as a transgene, e.g., therapeutic gene, reporter gene, or effector gene coding sequence in a vector. In addition, the vector (e.g., rAAV) polynucleotides may include additional elements, for example, a sequence encoding a reporter or a detectable marker, such as a fluorescent protein, or an element such as a Woodchuck Hepatitis Virus Post-transcriptional Regulatory Element (WPRE), which may increase RNA stability and protein yield. A vector (e.g., an rAAV) polynucleotide may also comprise a promoter to drive transcription of one or more polynucleotides (genes) which are inserted between inverted terminal repeats (ITRs). A polyadenylation signal, such as bovine growth hormone polyadenylation signal and/or SV40 polyomavirus simian virus 40 polyadenylation signal, may be included as elements in the vector (e.g., rAAV) polynucleotide. The vector (e.g., rAAV) polynucleotide can comprise a minimal promoter, e.g., a human beta-globin minimal promoter (phβg) and a chimeric intron sequence (Hermeming et al., 2004, J Virol Methods, 122 (1): 73-77). Without wishing to be bound by theory, ITRs may aid in concatamer formation in the nucleus after the single-stranded, AAV vector DNA (e.g., rAAV) is converted into double stranded (ds) DNA by host cell DNA polymerase complexes. Thus, the administration of the described rAAVs may form episomal concatemers in the nucleus of interneuron cells into which they are transduced. In non-dividing cells, such as adult interneurons, concatemers may remain intact in these cells for the lifetime of the interneurons. Advantageously, integration of vector (e.g., rAAV) polynucleotides into host chromosomes is likely to be negligible or absent and will not alter or affect the expression or regulation of any other human gene.

Recombinant AAV vectors can be made using standard and practiced techniques in the art and employing commercially available reagents. It will be appreciated by the skilled practitioner that rAAV vectors that been used in several clinical trials that have yielded promising results. By way of example, rAAV based therapy received marketing approval by the European Union in 2012, as reported by Kotterman, M. A. et al., 2014, Nat. Rev. Genet., 15:445-451. In some embodiments, plasmid vectors may encode all or some of the well-known replication (rep), capsid (cap) and adeno-helper components. The rep component comprises four overlapping genes encoding Rep proteins required for the AAV life cycle (e.g., Rep78, Rep68, Rep52 and Rep40). The cap component comprises overlapping nucleotide sequences of capsid proteins VP1, VP2 and VP3, which interact together to form a capsid of an icosahedral symmetry. A second plasmid that encodes helper components and provides helper function for the AAV vector may also be co-transfected into cells. The helper components comprise the adenoviral genes E2A, E4orf6, and VA RNAs for viral replication.

In an embodiment, a method of making rAAVs for the products, compositions, and uses described herein involves culturing cells that comprise an rAAV polynucleotide expression vector as described; culturing the cells to allow for expression of the polynucleotides to produce the rAAVs within the cell, and separating or isolating the rAAVs from cells in the cell culture and/or from the cell culture medium. Such methods are known and practiced by those having skill in the art. The rAAVs can be purified from the cells and cell culture medium to any desired degree of purity using conventional techniques.

In an embodiment, the rAAV vector contains an enhancer polynucleotide sequence as described herein and a chemogenetic DREADD (‘Designer receptor exclusively activated by designer drug’)-encoding sequence, e.g., a Gq-DREADD receptor (Hu, J. et al., 2016, J Biol Chem, 291:7809-7820). The amino acid sequence of the Gq-DREADD receptor has been reported by Armbruster et al. (2007, Proc Natl Acad Sci USA, 104:5163-5168). The amino acid sequence of the Gq-DREADD receptor is a derivative of the amino-acid sequence of the human muscarinic acetylcholine receptor, M3, in which the tyrosine in position 149 is replaced by a cysteine, and the arginine in position 239 is replaced by a glycine. The unmodified human sequence is provided under NCBI accession no. NP 000731.1. In an embodiment, the polynucleotide sequence that encodes the Gq-DREADD receptor in the rAAV vector can be modified, for example, by including optimized codons for expression of the Gq-DREADD receptor in human interneurons.

In an embodiment, the rAAV vector contains an enhancer polynucleotide sequence as described herein and a chemogenetic PSAM-encoding sequence.

Recombinant AAV vectors, which have a genome of small size (about 5 kb), can be engineered to package and contain larger genomes (transgenes), e.g., those that are greater than 4.7 kb. By way of example, two approaches developed to package larger amounts of genetic material (genes, polynucleotides, nucleic acid) include split AAV vectors and fragment AAV (fAAV) genome reassembly (Hirsch, M. L. et al., 2010, Mol Ther 18 (1): 6-8; Hirsch, M. L. et al., 2016, Methods Mol Biol, 1382:21-39). Split rAAV vector applications were developed to take advantage of the fact that rAAV genomes naturally concatamerize in the cell post-transduction and are substrates for enhanced homologous recombination (HR) (Hirsch, M. L. et al., 2016, Methods Mol Biol, 1382:21-39). This approach comprises “splitting” a large transgene into two separate vectors and upon co-transduction, intracellular large gene reconstruction via vector genome concatamerization occurs via HR or nonhomologous end joining (NHEJ). In general, three strategies exist within the split rAAV approaches: overlapping, trans-splicing, and hybrid trans-splicing.

Fragment AAV (fAAV) as an approach for AAV-mediated large gene delivery was developed based on reports that attempted encapsidation of transgenic cassettes exceeding the packaging capacity of the AAV capsid resulted in the packaging of heterogeneous single-strand genome fragments (<5 kb) of both polarities. After transduction by multiple fAAV particles, the genome fragments can undergo opposite strand annealing, followed by host-mediated DNA synthesis to reconstruct the intended oversized genome within the cell. (Hirsch, M. L. et al., 2016, Methods Mol Biol, 1382:21-39).

An advantage and benefit of the vectors, compositions and methods described herein is the identification and use of sufficiently small enhancer elements (cis-acting elements) that are capable of restricting or regulating gene expression in a defined population of cells, e.g., interneuron and neuron cell populations as described herein. In embodiments, the enhancer element is at least one of the enhancer sequences of SEQ ID NOs: 1-16 as described herein, which restrict gene expression to interneuronal and neuronal cell populations, such as, for example, inhibitory GABA-ergic neurons, such as parvalbumin (PV)-expressing interneurons, Somatostatin (SST)-expressing interneurons; Vaso-active Intestinal Peptide (VIP)-expressing interneurons; and non-VIP/CGE-derived interneurons (ID2).

The genes (transgenes) delivered by the rAAV vectors described herein are active and functional in the specific cells in which they are expressed, i.e., the products that they encode are produced, and are functionally expressed by the cells. By way of specific example, an rAAV vector as described herein which is engineered to contain an enhancer sequence that restricts expression of a transgene, e.g., reporter gene, to a GABA-ergic neuron (e.g., parvalbumin (PV)-expressing interneurons, Somatostatin (SST)-expressing interneurons; Vaso-active Intestinal Peptide (VIP)-expressing interneurons; and non-VIP/CGE-derived interneurons (ID2)), transduces these neuronal cell types, and the encoded reporter protein is functionally expressed in the GABA-ergic neuron cell type.

As another advantage, the described enhancer elements of any one of SEQ ID NOs: 1-16 that restrict, regulate, control, or modulate expression of genes in certain neuronal cell populations are of a size/length (kb), e.g., less than approximately 2 kb, to allow for their insertion in a rAAV vector along with other polynucleotide sequences, e.g., a transgene, effector gene, reporter polynucleotides, polynucleotides encoding DREADDs, and other effector proteins as described herein, e.g., CRISPR/Cas (e.g., Cas9), ZFPs, TALENS, etc. By way of example, given the obligate minimal size of reporter elements (e.g., Enhanced green fluorescent protein (EGFP), orange fluorescent protein (dTomato)), alone or in combination with effector or reporter elements, (e.g. Channelrhodopsin (ChR2), DREADDs), which average about 700 bp to 2 kb, respectively, a maximum of ~2 kb in packaging capacity remains for the insertion of a cis-acting DNA control element such as an enhancer sequence into an rAAV vector. The enhancer element sequences identified and described herein are capable of restricting expression to defined populations of neuronal cells and are sufficiently small elements to allow for additional nucleic acid sequences, reporter elements and transgenes, to also be cloned into the delivery vector, e.g., an AAV (rAAV) vector.

### Cell-Specific AAV Capsids

The rational design of AAV vectors that display selective tissue/organ targeting has broadened the applications of AAV as vector/vehicle for gene therapy. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature. (Chan, K. Y. et al., 2017, Nat. Neurosci., 20 (8): 1172-1179). Such vectors facilitate robust transduction of neuronal cells, including interneurons. When used with enhancers and cell-type specific promoters, such AAVs provide targeted gene expression in neuronal cells of the nervous system.

For applications that do not require high expression levels per cell, the amount of virus used, i.e., the viral dose, could be lowered. Lowering the viral load used for systemic gene delivery can reduce cost and production burden and minimize a potential risk for adverse reactions to viral components.

### Delivery of Recombinant Adeno-Associated Viral Vectors and Treatment Approaches

In general, the delivery of a transgene, e.g., a therapeutic gene, to treat a neurological, neurodevelopmental, neurodegenerative, neuropsychiatric, neurogenetic, or neuromuscular disease, disorder, or pathology at the genetic level, e.g., by modifying or correcting gene expression, such as by gene therapy, may be achieved using appropriate and effective vectors, such as viral or virus vectors, e.g., AAV or rAAV. By way of example, the use of a rAAV vector provides efficient delivery of therapeutic genes to a cell where the genes are expressed. While other methods and approaches for delivering genes to cells involve, for example, the use of purified DNA under hydrodynamic pressure, a shotgun approach using DNA adhering to gold particles, or lipid-DNA complexes, such methods and approaches frequently do not provide efficient gene delivery and result in gene expression that is lower than that required for therapeutic efficacy. Moreover, such methods are not applicable to human use. Viruses, on the other hand, represent natural vectors for the delivery and expression of exogenous genes in host cells in vivo.

An advantage associated with the use of rAAV as a viral vector is that rAAV transgene expression typically persists for years or for a lifetime, as has been demonstrated in animal models. This stands in contrast to non-rAAV viral vectors, which often lead to an initial burst of transgene expression that commonly disappears after a relatively short time, e.g., weeks.

To achieve enhanced therapy or treatment, the dose of rAAV vector that is required for a therapeutic response may be reduced, e.g., by using certain rAAV serotypes. Alternatively, the surface of the rAAV vector capsid may be altered to include specific ligands for attachment to target tissues and cells as described above. Another approach takes into consideration the trafficking of the virus particle from the endocytoplasmic vesicle to the nucleus. (Zhao, W. et al., 2007, Gene Ther., 14:545-550; Daya, S. and Berns, K. I., 2008, Clin. Microbiol. Rev., 21 (4): 583-593). Typically, the virus particle-to-infectivity ratio of rAAV vector preparations ranges from 10:1 to 100:1. The high ratios reflect incomplete or empty vector particles, as well as trafficking from the endocytoplasmic vesicle to the nucleus. During trafficking, the vector particle may become ubiquitinated and directed to a proteasome for degradation, rather than to the nucleus where the transgene may be expressed. It was found that ubiquitination and direction to the proteasome require phosphorylation of tyrosine residues on the surface of the rAAV vector capsid. When the seven tyrosine residues on the surface of the AAV-2 capsid were replaced phenylalanine residues, the multiplicity of infection (MOI) required for the detection of transgene expression was greatly reduced both in cell culture and in several mouse models of transduction of cells in the liver and eye. Consequently, the ability to increase transgene expression to therapeutic levels in the treatment of diseases may be enhanced.

For direct delivery to the brain, rAAV vectors may be administered by open neurosurgical procedure or by focal injection in order to bypass the blood-brain barrier, to restrict transgene expression temporally and spatially, and to target specific areas of the brain, e.g., brain cortex or brain tissue (e.g., cortical tissue) comprising specific populations of neuron and interneuron cells.

Systemic rAAV delivery (by intravenous injection) provides a non-invasive alternative for broad gene delivery to the nervous system; however, the high viral load required and relatively low transduction efficiency have limited wide adoption of this method. Several groups have developed rAAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BRI capsid20, based on AAV2, may be useful for more efficient and selective transduction of brain endothelial cells. Another AAV capsid, AAV-PHP.B, comprises a capsid that transduces the majority of neurons and astrocytes across many regions of the adult mouse brain and spinal cord after intravenous injection. In an embodiment, rAAV comprises a capsid which specifically transduced interneurons, including PV interneurons, in the cerebral cortex (brain).

Other modes of rAAV vector administration may include lipid-mediated vector delivery, hydrodynamic delivery, and a gene gun. In a particular embodiment, the rAAV vectors comprise a capsid that increases the likelihood of directly infecting or transducing interneuron cells, such as GABA-ergic interneuron cells, such as PV-expressing interneurons, Somatostatin (SST)-expressing interneurons, Vaso-active Intestinal Peptide (VIP)-expressing interneurons, and non-VIP/CGE-derived interneurons (ID2), and brain tissue comprising these cells.

### Method of Identifying Enhancer Sequences for Targeting Distinct Cortical Neuron and Interneuron Cells and Cell Subtypes

A method of identifying enhancer sequences for targeting distinct cortical neuronal and interneuronal cells and cell subtypes is provided and described, e.g., Example 1. To create the method, systematic analysis of single cell ATAC-seq and RNA-seq data was performed to identify enhancer candidates for each of the cortical interneuron subtypes. Candidate enhancer sequences were screened to successfully identify enhancer sequences that were highly selective for distinct cortical interneuron populations in mouse and non-human primates. These enhancer sequences, when used in the context of different effector genes, can target specific neuronal subtypes (e.g., using fluorescent reporter proteins), observe activity (gCaMP), manipulate (opto- or chemo-genetics) and trace the connectivity (used in conjunction with mono-synaptic rabies) of specific neuronal subtypes. The neuronal cell targeting enhancer sequences contained in rAAV vectors, e.g., as viral vector tools, were also validated across species. Described herein are the development of a method of enhancer identification and related validation strategy for identifying and producing tissue/cell-type specific enhancer sequences that target particular cortical neurons and interneurons and the neural circuits to which they contribute, in particular, for therapeutic use.

As described herein, a method was developed to identify with accuracy cell type-specific enhancers to target different neuronal cell types; the method is termed “PIASO (Precise Integrative Analysis of Single-cell Omics)” herein. Distinguishing features of the method include improved peak calling to identify cell type-specific enhancers, optimized integration of single-cell RNA-seq (scRNA-seq) and single-cell ATAC-seq (scATAC-seq) data, and prioritization of enhancers by accessibility pattern conservation across species.

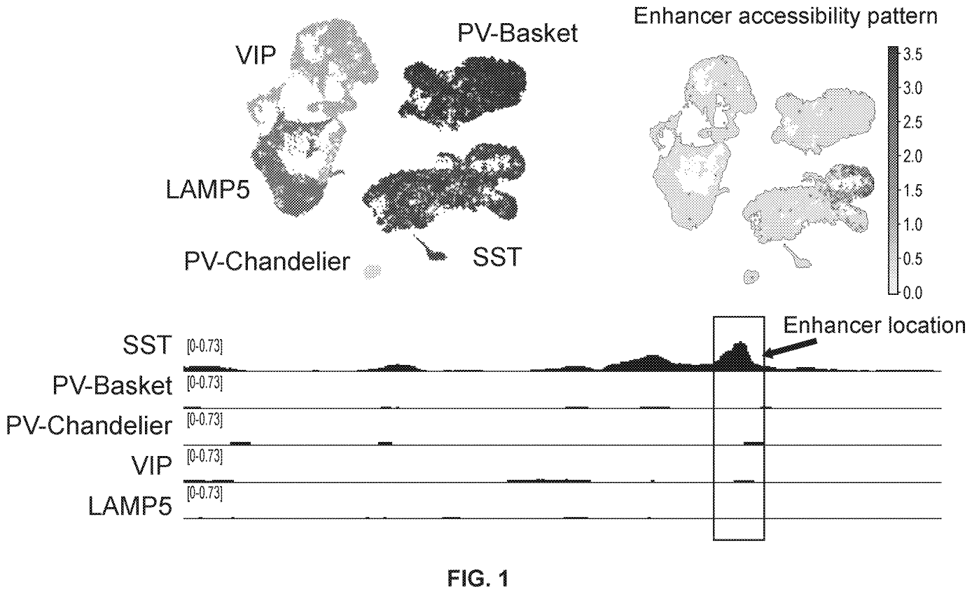

To identify subtype-specific accessible genomic regions from scATAC-seq data, the method efficiently quantifies the accessibility of small-size (e.g., 500 bp) bins of genomic regions with optimized data structure and algorithm. The method projects all bins into a cell space and compares each bin to ideal cell type-specific bins (like anchors in the cell space) to identify top subtype-specific bins. Next, the method provides call peaks using these sparse signals from the subtype-specific bins with limited cell numbers. After that, the method merges these peaks with peaks called from individual cell types and peaks called from all cells as one union peak set. This peak set was then used for accurate analysis of cell population heterogeneity and thereafter cell type-specific enhancer identification (FIG. 1). The specificity of nominated enhancer sequences was computationally validated with bulk ATAC-seq data and bulk ChIP-seq data from enriched cell populations.

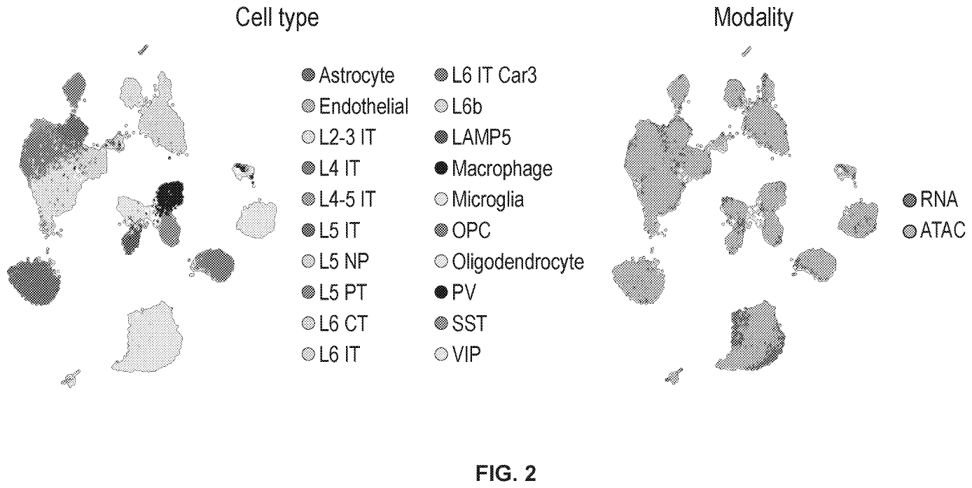

The method significantly improved integration of unpaired scRNA-seq and scATAC-seq data. (FIG. 2). This allowed for accurate assignment of subtype identity for cells in scATAC-seq data based on the marker gene expression patterns obtained from scRNA-seq data. Most current methods impute gene activity from scATAC-seq data and in doing so, often lose signals from cell type-specific accessible regions. The method described herein directly integrates different modalities (i.e., RNA and ATAC) of cells via “cell identity feature space,” where each axis represents one specific cell identity feature. The method preserves the specificity information for both modalities, i.e., marker genomic regions for ATAC and marker genes for RNA, and uses the region-gene associations as the “feature tunnel” to project cells from one modality onto the other modality's “cell identity feature space.” Cells then have the low-dimensional representations after the RNA space and ATAC space are concatenated.

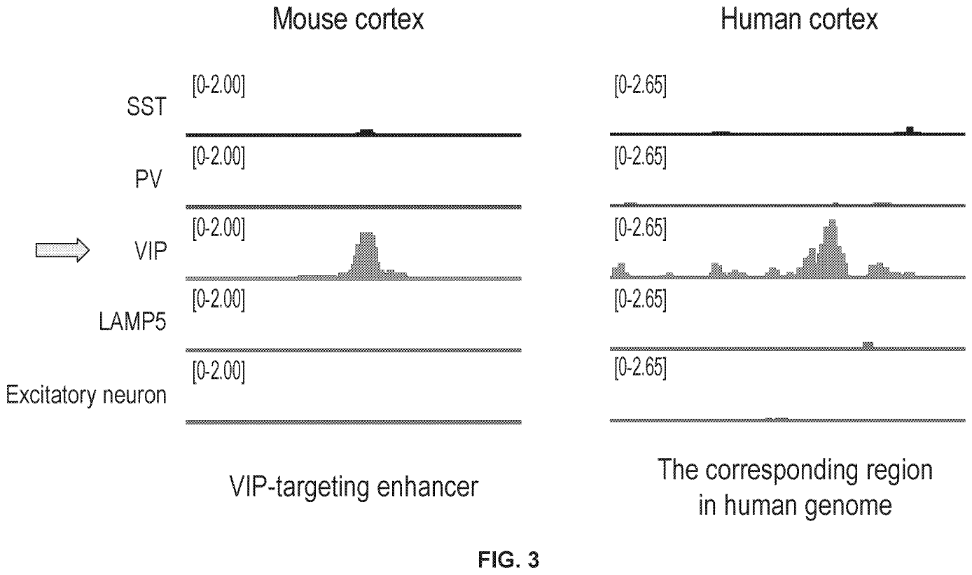

The method described herein also takes into consideration whether cell type-specific enhancers identified in mouse scATAC-seq data have the conserved cell type-specific accessibility pattern in human scATAC-seq data (FIG. 3).

In accordance with the foregoing, a computer-implemented method for cell type-specific enhancer identification is provided, in which the method involves the steps of a) quantifying peaks by analyzing single cell Assay of Transposase Accessible Chromatin sequencing (scATAC-seq) data to call peaks present in cell subtypes; b) analyzing the quantified peaks present in the scATAC-seq data of a) to identify cell type specific enhancers; c) assigning cell type identity comprising integrating the scATAC-seq data of b) and single-cell RNA-seq (scRNA-seq) data, wherein marker expression patterns in the scRNA-seq data provide for the assignment of cell type identity for cells in the scATAC-seq data; and d) validating cell type specific enhancers comprising identifying regions of conservation across species in cell type-specific accessibility patterns present in cell type specific enhancers. In an embodiment, prior to peak quantification, the method comprises quantifying the accessibility of bins of genomic regions and projecting the bins into a cell space. In an embodiment, the bins comprise genomic regions of approximately 500 base pairs (bp). In an embodiment, the method further involves comparing each bin to an ideal cell type-specific bin to prioritize sub-type specific bins that most closely resemble the ideal cell type-specific bin. In an embodiment, the prioritized sub-type specific bins are used in the peak quantification of step (a) of the method delineated above. In an embodiment, the peaks are merged with peaks called from individual cell types and peaks called from all cells to generate a union peak set. In an embodiment, the union peak set is used to determine cell population heterogeneity prior to carrying out step (b) of the above-delineated method. In an embodiment, the scRNA-seq and scATAC-seq data are obtained from one or more neurons or interneurons. In an embodiment, the neurons are present in brain cortex or cortical tissue. In an embodiment, the neurons or interneurons are somatostatin (SST)-expressing interneurons, inhibitory interneurons, Parvalbumin (PV)-expressing basket cells, PV-expressing chandelier interneurons, Vasoactive intestinal polypeptide (VIP)-expressing interneurons, or Lamp5-expressing interneurons. In an embodiment, the method further involves the step of displaying or translating the results or output of the steps in a visual form. In an embodiment, the step of displaying or translating includes displaying the results or output on a display device. In an embodiment, the display device is a desktop computer, a laptop computer, a hand-held computer, a smart phone, a cellular telephone, a tablet computer, or a personal digital assistant.

### Treatment and Therapy for Neurological Diseases, Pathologies and Conditions

The enhancer element sequences described herein provide advantages and benefits for targeting the expression of genes in particular neuronal cell types, especially when the enhancer element sequence is a component of a delivery vector, such as a lentivirus vector, an adeno-associated virus (AAV) vector, or a recombinant adeno-associated virus (rAAV) vector, which targets a particular neuronal cell type in which the gene is expressed and functional. In an embodiment, the expressed gene is a therapeutic gene, which may treat, ameliorate, improve, reduce, abate, diminish, resolve, or eliminate a disease, pathology, or condition, or a symptom thereof, when expressed in a particular neuronal cell type, which expresses an aberrant or mutated form of the gene, or in which the gene is nonfunctional or not expressed due to loss, mutation, silencing, non- or dysfunction, and the like. In an embodiment, the genes are those associated with or causative of a particular disease, pathology, or condition, such as a neuronal, neurological, neurodevelopmental, neurodegenerative, neuropathological, neurogenetic, neuropsychiatric, or neuromuscular disease, pathology or condition, and/or symptoms thereof. Nonlimiting examples of such diseases, pathologies, or conditions include epilepsy, seizures, Alzheimer's disease, Parkinson's disease, Dystonia, Amyotrophic lateral Sclerosis (ALS), and Down Syndrome.

### Pharmacogenetic Approaches

Pharmacogenetic approaches are contemplated for use with the virus vectors, rAAV vectors, compositions thereof, and methods described herein. Such approaches deliver either Gq-DREADD receptor or PSAM into PV-interneurons specifically using a viral vector, such as a rAAV vector comprising an enhancer element (e.g., ME1, HuE1, ME2, HuE2) as described herein and a polynucleotide encoding a Gq-DREADD receptor or PSAM. The targeted PV-neurons, either in a specific region upon focal injection or throughout the cortex upon systemic injection, as dictated by the type of pathology being treated, stably express the receptor (Gq-DREADD or PSAM). Thereafter, an individual (patient) is administered the drug that activates the receptor (e.g. CNO or PSEM, respectively). This approach results in a controlled alteration of the excitability of the PV-interneurons expressing the receptor and allows for a dose-dependent and time-dependent modulation of the excitation/inhibition (E/I) balance in neurons (interneurons and PV-expressing interneurons), resulting in a normalization of brain activity. Pharmacogenetic approaches using the enhancer element sequences described herein are also contemplated for regulating or modulating gene expression and function in other types of neurons, such as SST and VIP interneurons.

### Pharmaceutical Compositions

Provided also are pharmaceutical compositions or formulations for treating subjects who are afflicted with, or who are at risk of developing, a neurological, neurodevelopmental, neurodegenerative, neuropsychiatric, neurogenetic, or neuromuscular disease, disorder, or pathology. In an embodiment, the pharmaceutical composition includes an AAV vector or virus particle, such as one containing an enhancer sequence as described herein (as active agent) and a pharmaceutically acceptable carrier, excipient, or diluent. When formulated in a pharmaceutical composition, an rAAV vector as therapeutic compound or product can be admixed with a pharmaceutically acceptable carrier, diluent, or excipient.

The therapeutic agent(s) may be contained in any appropriate amount in any suitable carrier substance, and is/are generally present in an amount of 1-95% by weight of the total weight of the composition. The composition may be provided in a dosage form that is suitable for a parenteral (e.g., subcutaneous, intravenous, intramuscular, or intraperitoneal) administration route, such that the agent, such as a viral vector described herein, is systemically delivered. In an embodiment, systemic injection of an rAAV vector as described herein allows for the characterization of specificity of expression across brain regions, particularly when a reporter product is also encoded by the vector. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may be formulated to release the active agent substantially immediately upon administration or at any predetermined time or time after administration. The latter types of compositions are generally known as controlled release formulations, which include (i) formulations that create a substantially constant concentration of the agent within the body over an extended period of time; (ii) formulations that after a predetermined lag time create a substantially constant concentration of the drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with a target site or location, e.g., in a region of a tissue or organ; (v) formulations that allow for convenient dosing, such that doses are administered, for example, once every one, two, or several weeks; and (vi) formulations that target a specific tissue or cell type using carriers, chemical derivatives, or specifically designed vectors (e.g., comprising a certain capsid composition) to deliver the therapeutic agent, e.g., to interneurons or PV-, or SST-, or VIP-, or Lamp5-expressing GABA-ergic interneurons. For some applications, controlled release formulations obviate the need for frequent dosing during the day to sustain the plasma level of the administered agent at a therapeutic level.

Methods by which to obtain controlled release in which the rate of release outweighs the rate of metabolism of the agent in question are not meant to be limiting. By way of example, controlled release is obtained by appropriate selection of various formulation parameters and ingredients, including, e.g., various types of controlled release compositions and coatings. Thus, the therapeutic agent is formulated with appropriate excipients into a pharmaceutical composition that, upon administration, releases the agent in a controlled manner. Examples include single or multiple unit tablet or capsule compositions, oil solutions, suspensions, emulsions, microcapsules, microspheres, molecular complexes, nanoparticles, patches, and liposomes.

The administration of a composition comprising a combination of agents for the treatment or therapy of a neurological, neurodevelopmental, neurodegenerative, neuropsychiatric, neurogenetic, or neuromuscular disease, disorder, pathology, or condition may be by any suitable means that results in a concentration of the therapeutic that, combined with other components, is effective in ameliorating, abating, reducing, decreasing, or stabilizing seizures in a subject. The composition may be administered systemically, for example, formulated in a pharmaceutically-acceptable buffer such as physiological saline. In an embodiment, systemic injection of an rAAV vector as described herein allows for the characterization of specificity of expression across brain regions, particularly when a reporter product is also encoded by the vector.

Routes of administration include, for example, intracranial, parenteral, subcutaneous (s.c.), intravenous (i.v.), intraperitoneal (i.p.), intramuscular (i.m.), or intradermal administration, e.g., by injection, that optimally provide continuous, sustained levels of the agent in the patient. The amount of the therapeutic agent to be administered varies depending upon the manner of administration, the age, physical condition and body weight of the patient, and with the clinical symptoms of the neurological, neurodevelopmental, neurodegenerative, neuropsychiatric, neurogenetic, or neuromuscular disease, disorder, or pathology. Generally, amounts will be in the range of those used for other viral vector-based agents employed in the treatment of neurological, neurodevelopmental, neurodegenerative, neuropsychiatric, neurogenetic, or neuromuscular diseases, disorders, pathologies and conditions, particularly in the brain, although in certain instances lower amounts are needed if the agent exhibits increased specificity. A composition is administered at a dosage that shows a therapeutic effect, such as, for example, ameliorating, abating, reducing, decreasing, or stabilizing seizures in a patient, as determined by methods known to one skilled in the art.

The pharmaceutical composition may be administered parenterally by injection, infusion or implantation (subcutaneous, intravenous, intramuscular, intraperitoneal, intracranial, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation, and can be found, for example, in Remington: The Science and Practice of Pharmacy, supra. In particular embodiments, administration is systemic and parenteral, such as by injection or intravenous delivery.

Compositions for parenteral delivery and administration may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (e.g., viral vector or particle comprising enhancer sequences and polynucleotides encoding a transgene, e.g., a therapeutic gene or an effector gene, and associated regulatory sequences, as described herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic(s) (i.e., viral vector or particle described herein) is formulated for intravenous delivery. As noted above, the pharmaceutical compositions according to the described embodiments may be in the form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Methods of Administration and Delivery

Administration of a viral vector or pharmaceutical composition as described herein to a subject, e.g., a patient having, or at risk of having, a neurological, neurodevelopmental, neurodegenerative, neuropsychiatric, neurogenetic, or neuromuscular, disease, disorder, or pathology, and/or the symptoms thereof. In embodiments, the viral vector, viral particle, or pharmaceutical composition may be delivered to a cell (e.g., a target cell such as an interneuron or a brain layer comprising interneurons) in any manner such that the viral vector, particle or composition is functional and active to express the sequences contained in the vector or virus particle. Illustratively, rAAV comprising an enhancer element as described herein and a gene or polynucleotide sequence of interest (e.g., a therapeutic gene) may be delivered to interneuron or neuron cells or tissue comprising interneuron or neuron cells to provide for targeted expression of the gene (and the encoded gene product) in the interneurons or neurons. Thus, viral vectors or viral particles are delivered to a cell by contacting the cell with a composition comprising the viral vectors, or viral particles and by heterologous expression of the polynucleotides harbored in the viral vector or viral particles in the cell. The polynucleotides harbored in the rAAV vector must be delivered to the cells of a subject in a form in which they can be taken up so that therapeutically effective levels of the encoded products can be produced.

Transducing rAAV vectors are used for the delivery and expression of genes encoding desired proteins, polypeptides, or peptides to cells, especially because of their high efficiency of infection and stable integration and expression (see, e.g., Cayouette et al., Human Gene Therapy, 8:423-430, 1997; Kido et al., Current Eye Research, 15:833-844, 1996; Bloomer et al., Journal of Virology, 71:6641-6649, 1997; Naldini et al., Science, 272:263-267, 1996; and Miyoshi et al., Proc. Natl. Acad. Sci. U.S.A., 94: 10319, 1997). By way of example, rAAV is engineered to contain a polynucleotide encoding a specific enhancer nucleic acid sequence as described herein that preferentially directs gene expression in interneuron cell types and is used to direct and restrict the expression of a gene in GABA-ergic interneuron target cells. In an embodiment, expression of the gene of interest in a given cell can be driven from any suitable promoter, such as a promoter specific for the target cells. In an embodiment, the rAAV vector is administered systemically. In an embodiment, systemic injection of an rAAV vector as described herein allows for the characterization of specificity of expression across brain regions, particularly, for example, when a reporter product is also encoded by the vector.

Gene transfer can also be achieved using in vitro transfection methods. Such methods include the use of calcium phosphate, DEAE dextran, electroporation, and protoplast fusion. Liposomes can also be potentially beneficial for delivery of DNA into a cell.

### Treatment Methods and Protocols

Provided are methods of administering a therapeutic agent to a subject in need, such as a subject having, undergoing, having experienced, and/or at risk of experiencing a neurological, neurodevelopmental, neurodegenerative, neuropsychiatric, neurogenetic, or neuromuscular disease, disorder, or pathology, more particularly, a seizure or epilepsy, and who also may be diagnosed with, or be suspected of having, or having symptoms of, a seizure disorder, or who is identified as being in need of such treatment, in which an effective amount of a viral vector or viral particle as described herein, or a composition described herein, is administered to the subject to produce a therapeutic effect.

According to the described methods, a therapeutic effect includes, without limitation, that amount of rAAV that is introduced into a sufficient number of interneurons so as to inhibit, reduce, abate, abrogate, or ameliorate one or more symptoms of the neurological, neurodevelopmental, neurodegenerative, neuropsychiatric, neurogenetic, or neuromuscular disease, disorder, or pathology, e.g., a seizure or epilepsy, or to prevent one or more symptoms subsequent to the administration of the rAAV vector product or composition to the subject. The amount of rAAV that is administered may be determined by the skilled practitioner in the art, such as a medical or clinical practitioner, and, as appreciated by one skilled in the art, is based on factors such as the size of the epileptic focus, the titer of the virus preparation and from data acquired in non-human primates (e.g., Colle, M.-A. et al., 2010, Hum. Mol. Genet., 19:147-158). By way of example, from 10¹⁰ to 10¹² rAAV particles may be used to transduce a therapeutically relevant number of interneurons with rAAV vectors or particles thereof. Identifying a subject in need of such treatment can be in the judgment of a subject or a health care professional and can be subjective (e.g. opinion) or objective (e.g. measurable by a test or diagnostic method).

The therapeutic methods (which include prophylactic treatment) in general comprise administration of a therapeutically effective amount of the agents described herein, such as an rAAV vector, a viral particle, or composition containing the aforementioned agents, to a subject (e.g., animal, mammal, non-human primate, or human) in need thereof, including a mammal, particularly a human. Such treatment will be suitably administered to subjects, particularly humans or infant humans, suffering from, having, susceptible to, or at risk for a neurological, neurodevelopmental, neurodegenerative, neuropsychiatric, neurogenetic, or neuromuscular disease, disorder, or pathology, such as seizures and/or epilepsy. Determination of those subjects “at risk” can be made by any objective or subjective determination by a diagnostic test or opinion of a subject or health care provider (e.g., genetic test, enzyme or protein marker or biomarker, family history, and the like).

Viral vectors and pharmaceutical compositions as described can be used therapeutically to treat patients suffering from neurological, neurodevelopmental, neurodegenerative, neuropsychiatric, neurogenetic, or neuromuscular diseases or disorders, e.g., seizures, epilepsy, etc., or prophylactically to provide advanced treatment or protection to patients at risk for certain neurological, neurodevelopmental, neurodegenerative, neuropsychiatric, neurogenetic, or neuromuscular diseases, disorders, or pathologies, such as a prophylactic vaccination to reduce, diminish, abate, or ward off one or more symptoms of the disease, disorder, or pathology, and/or the severity thereof. A prophylactically effective amount of the rAAV vectors as described herein are not intended to be limiting herein, and may range between about 10² TU (transducing units) per kilogram body weight of the recipient and about 10²⁰ TU kilogram body weight of the recipient, or any TUs in between those values. Mouse models of disease or disorders, e.g., seizures, can be used to optimize dosages and regimens.

The therapeutic vectors as described herein may be administered to a subject in need thereof in an effective amount to normalize the excitability of certain genes associated with particular types of interneurons or neurons that may be deficient in a functioning gene to alleviate a neurological, neurodevelopmental, neurodegenerative, neuropsychiatric, neurogenetic, or neuromuscular disease, disorder, pathology, or condition and/or the symptoms thereof. The vectors and methods described herein may be of therapeutic value for an individual, e.g., a human infant, child or adult, who experiences or is at risk for experiencing one or more symptoms of the disease, disorder, or pathology. In an embodiment, an rAAV vector or a composition comprising an rAAV vector as described herein is administered to an individual whose interneurons do not express or exhibit loss of function or expression, at the time of administration, of a certain neuronal- or interneuronal cell gene, which is dependent for expression on an enhancer element, such as ME1-ME8 of SEQ ID NOs: 1-8 and their human counterparts HuE1-HuE8 of SEQ ID NOs: 9-16, respectively, as described herein. In an embodiment, the expression of a therapeutic gene in interneuron or neuron cells transduced by the described rAAV vectors containing an enhancer sequence that targets or restricts expression of the gene in certain cell types normalizes the excitability of interneurons or neurons that are deficient in, or have abnormal expression of, the gene. In an embodiment, a composition comprising an rAAV vector as described herein is administered to an individual whose interneurons no longer express a given gene. In an embodiment, a composition comprising an rAAV vector as described herein is administered to an individual of any age, e.g., infant to adult.

Subjects, e.g., mammalian subjects, and human patients to whom the rAAV vectors as described herein are administered may also benefit from adjunct or additional treatments, therapeutic compounds, drugs and/or surgical techniques, as are well known to those having skill in the art, to assist in or to augment the therapy and treatment of the disease, disorder, or pathology.

### Kits

Also provided are kits for preventing or treating a neurological, neurodevelopmental, neurodegenerative, neuropsychiatric, neurogenetic, or neuromuscular disease, disorder, pathology, or condition, and/or the symptoms thereof in a subject in need thereof, including humans and non-human mammals. In one embodiment, the kit provides a therapeutic or prophylactic composition containing an effective amount of a rAAV vector or viral particle as described herein, which comprises an enhancer polynucleotide sequence specific for a given gene that targets or restricts the expression of the gene, e.g., contained in the virus vector, to interneuron or neuron cells of certain types as described herein, including GABA-ergic interneuron cells in the brain (e.g., in the telecephalon) or brain cortex. In an embodiment, the enhancer element is a mouse or human sequence of any one of SEQ ID NOs: 1-16. In an embodiment, the enhancer is one or more of SEQ ID NOs: 1, 2, 9, or 10, as described herein, which restricts expression of a gene of interest (e.g., a therapeutic gene, reporter gene, or an effector gene, e.g., a polynucleotide encoding a Gq-DREADD or PSAM for chemogenetic modulation of PV-interneuron activity, or genes or polynucleotides encoding CRISPR-Cas9, Zinc Finger Protein, TALENs, or engineered or variant forms thereof, to PV-expressing interneuron cells (e.g., basket or chandelier PV interneurons). In an embodiment, the enhancer is one or more of SEQ ID NO: 3 or 11, as described herein, which restricts expression of a gene of interest to SST-expressing interneuron cells. In an embodiment, the enhancer is one or more of SEQ ID NO: 8 or 16, as described herein, which restricts expression of a gene of interest to VIP-expressing interneuron cells. In an embodiment, the enhancer is one or more of SEQ ID NOs: 4-7 or 12-15, as described herein, which restricts expression of a transgene to Lamp5-expressing interneurons.

In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A composition comprising an rAAV vector comprising at least an enhancer polynucleotide sequence as described herein is provided together with instructions for administering the composition to a subject having or at risk of developing a neurological, neurodevelopmental, neurodegenerative, neuropsychiatric, neurogenetic, or neuromuscular disease, disorder, pathology, or condition, and/or the symptoms thereof. In an embodiment, the rAAV vector comprises a gene of interest (e.g., a therapeutic gene or a transgene) for expression in interneuron cells including inhibitory GABA-ergic interneurons. The instructions will generally include information about the use of the composition for the treatment or prevention of the disease, disorder, pathology, and/or the symptoms thereof. In other embodiments, the instructions include at least one of the following: description of the therapeutic agent (rAAV comprising an enhancer polynucleotide sequence, etc.); dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Further Embodiments and Advantages Thereof

In other embodiments, the enhancers identified and described herein provide access to particular cell populations with distinct clinical relevance. By way of example, these enhancers be used to alleviate the debilitating aspects of a number of neurological, neurodevelopmental, neurodegenerative, neuropsychiatric, neurogenetic, or neuromuscular diseases, disorders, or pathologies, either through gene therapy or via modulation of neuronal activity, e.g., via optogenetic or chemogenetic approaches. (See, e.g., Walker, M. C. et al., 2019, Neuropharmacology, 107751. doi: 10.1016/j.neuropharm.2019.107751. Review. PMID: 31494141). As described and demonstrated herein, local and systemic injections can be used for effective viral delivery to the brain, thus providing delivery and administration methods for clinical interventions. By way of example, local injections (e.g., of recombinant virus carrying an enhancer element and target polynucleotide, as well as other sequences included for expression of the plasmid contents in cells) may be employed to alleviate focal epilepsy, prefrontal cortex dysfunction or hippocampal memory disorders. Systemic administration or delivery of virus may be employed in contexts where global interventions are necessary, for example, to correct generalized seizures or for psychiatric and neurodegenerative disorders. As provided by the embodiments described and exemplified herein, the rigorous identification of enhancer elements (enhancer regulatory elements) allows for accessing target neuronal cell types. Such elements are advantageous for use in both experimental and therapeutic procedures and methods.

Display of Results and Output and/or Parameters of Method Steps in a Visual Form, Such as on a Display Device Involving Computer Implementation

In an aspect related to the described method, a non-transitory computer readable medium containing program instructions executable by a processor is provided, in which the computer readable medium contains program instructions that provide criteria that relate one or more parameters to each other, the parameters including one or more selected from the group consisting of: quantifying peaks by analyzing single cell Assay of Transposase Accessible Chromatin sequencing (scATAC-seq) data to call peaks present in cell subtypes; analyzing the quantified peaks present in the foregoing scATAC-seq data to identify cell type specific enhancers; assigning cell type identity comprising integrating the scATAC-seq data of the analyzing step and single-cell RNA-seq (scRNA-seq) data, wherein marker expression patterns in the scRNA-seq data provide for the assignment of cell type identity for cells in the scATAC-seq data; and validating cell type specific enhancers comprising identifying regions of conservation across species in cell type-specific accessibility patterns present in cell type specific enhancers, and programming instructions that input parameters of the method steps into given criteria and relate observations for one or more acquired parameters; and program instructions that converge the given criteria so as to provide an output representative of cell type-specific enhancer identification.

The method described herein provides the identification of enhancer sequences based on the analyses of RNA isolated from single cells. The identified enhancer sequences allow for expression of a gene or polynucleotide of interest in a particular neuronal or interneuronal cell type or subtype. In an aspect, the method involves displaying the input, output, and results of inputted data in one or more, or all, of the steps of the method so that sequence information obtained using scATAC-seq and RNA seq analyses can be visualized, e.g., in peaks (“bins”) of sequences. Sequence analysis and the results thereof carried out in the performance of the method can be embodied in computer program software for execution on a computer, digital processor, or microprocessor. Those skilled in the art will appreciate that the method involves computer program code elements, including logic circuits on an integrated circuit that function according to the described method. As such, one or more, or all, of the steps of the method may be practiced, with human input and involvement, by a machine component that renders the program code elements in a form that instructs a digital processing apparatus (e.g., computer) to perform a sequence of function step(s) corresponding to those carried out in the method.

Accordingly, the method as described herein is suitable for use in combination with any of a number of computer systems as are known to those skilled in the art or hereinafter developed. Such a computer system typically includes a computer, a display, and one or more input device(s). The display is any of a number of devices known to those skilled in the art for displaying images responsive to output signals from the computer, including but not limited to, cathode ray tubes (CRT), liquid crystal displays (LCDS), plasma screens and the like. The signals being outputted from the computer can originate from any of a number of devices including PCI or AGP video boards or cards mounted with the housing of the computer that are operably coupled to the computer's microprocessor and the display.

The one or more input device(s) are any of a number of devices known to those skilled in the art that can be used to provide input signals to the computer for control of applications programs and other programs such as the operating system (OS) being executed within the computer. Illustratively, the input device may comprise a switch, a slide, a mouse, a track ball, a glide point or a joystick, or other such device (e.g., a keyboard having an integrally mounted glide point or mouse) by which a user can input control signals other than by means of a keyboard.

The computer typically includes a central processing unit (CPU) including one or more microprocessors such as those manufactured by Intel or AMD, Motorola or the like, random access memory (RAM), mechanisms and structures for performing I/O operations, a storage medium such as a magnetic hard disk drive(s) or other drives (fixed or removable) for storage of data, operating systems or the applications or software programs associated with the methods, including an applications program and a device for reading from and/or writing to a removable computer readable medium, such as, for example, an optical disk reader capable of reading CDROM, DVD or optical disks and readers of other types of nonvolatile memory, such as flash drives, jump drives, or spin memory that embody one or more types of non-volatile memory or storage devices.

Such a hard disk drive serves to boot or store the operating system, other applications, or systems that are to be executed on the computer, paging and swapping between the hard disk and the RAM and the like. Such data also can be stored in a removable computer readable medium such as a CD or DVD type of medium that is inserted into a device for reading and/or writing to the removable computer readable media. Alternatively, such a computer system also includes a network based computer system that includes a server, an external storage device and a network infrastructure that operably couples a plurality or more of client computer systems to the server.

The server is any of a number of servers known to those skilled in the art that are intended to be operably connected to a network so as to operably link a plurality of client computers via the network to the server and thus also to the external storage device. Such a server typically includes a central processing unit including one or more microprocessors such as those manufactured by Intel or AMD, random access memory (RAM), mechanisms and structures for performing I/O operations, a storage medium such as a magnetic hard disk drive(s), and an operating system for execution on the central processing unit.

Devices suitable for the display of data generated by the described methods include desktop computers, laptop computers, handheld computer devices, smart phone, cellular telephone, tablet computer, or personal digital assistant. Such devices typically have an OS capable of running application software (e.g., Apps) and also typically provide for wireless connection to the Internet (e.g., WI-FI, Bluetooth).

The practice of the described embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides, viral vectors and viral particles and, as such, may be considered in making and practicing the embodiments described herein. Particularly useful techniques for particular embodiments will be discussed in the sections that follow.

The following examples are put forth so as to provide those of ordinary skill in the art with a complete disclosure and description of how to make and use the products, compositions and therapeutic methods as described herein, and are not intended to limit the scope of what is described and exemplified herein.

### EXAMPLES

### Example 1—Identification of Enhancer Sequences that Target Specific Interneuronal Cell Types, Subtypes and Populations

Described in this Example is a method for accurately identifying cell type-specific enhancers to target different cell types, namely, neuronal cell types and subtypes. The method, termed PIASO (“Precise Integrative Analysis of Single-cell Omics”) herein, features improved peak calling to identify cell type-specific enhancers, optimized integration of single-cell RNA-seq (scRNA-seq) and single-cell ATAC-seq (scATAC-seq) data, and prioritization of enhancers by accessibility pattern conservation across species.

To identify subtype-specific accessible genomic regions from scATAC-seq data, the method efficiently quantified the accessibility of small-size (e.g., 500 bp) bins of genomic regions with optimized data structure and algorithm. The method projected all bins into a cell space and compared each bin to ideal cell type-specific bins (like anchors in the cell space) to identify top subtype-specific bins. Next, the method used a newly developed strategy to call peaks using these sparse signals from the subtype-specific bins with limited cell numbers. After that, the method merged these peaks with peaks called from individual cell types and peaks called from all cells as one union peak set. This peak set was then used for accurate analysis of cell population heterogeneity and thereafter cell type-specific enhancer identification (FIG. 1). The specificity of nominated enhancers was computationally validated with bulk ATAC-seq data and bulk ChIP-seq data from enriched cell populations.

The method significantly improved integration of unpaired scRNA-seq and scATAC-seq data, which allowed for accurate assignment of subtype identity for cells in scATAC-seq data based on the marker gene expression patterns from scRNA-seq data. While most current methods impute gene activity from scATAC-seq data and in doing so, often lose signals from cell type-specific accessible regions, the method used herein directly integrated different modalities (i.e., RNA and ATAC) of cells via a “cell identity feature space,” in which each axis represents one specific cell identity feature. FIG. 2 shows that the method accurately integrates scRNA-seq data and scATAC-seq data collected from adult mouse cortex. The method preserves the specificity information for both modalities, i.e., marker genomic regions for ATAC and marker genes for RNA, and uses the region-gene associations as the “feature tunnel” to project cells from one modality onto the other modality's “cell identity feature space.” Cells then had the low-dimensional representations after the RNA space and ATAC space were concatenated.

The method also takes into consideration whether cell type-specific enhancers identified in mouse scATAC-seq data have the conserved cell type-specific accessibility pattern seen in human scATAC-seq data (FIG. 3).

Using this method, the 8 mouse enhancer sequences (SEQ ID NOs: 1-8) and their human orthologs/counterparts (SEQ ID NOs: 9-16, respectively) as described herein were identified and discovered, thus providing the successful identification of new enhancer elements that targeted specific interneuron populations in the brain cortex. (FIGS. 5A-5H). As described herein, the identified enhancer sequences were determined to target the following distinct classes of neurons and interneurons: parvalbumin-expressing interneurons (PV interneurons), e.g., PV-expressing chandelier interneurons and PV-expressing basket interneurons, which are fast-spiking neurons; Somatostatin (SST)-expressing interneurons

(SST interneurons), Vaso-active Intestinal Peptide expressing interneurons (VIP interneurons) and Lamp5-expressing interneurons. The identified enhancer sequences were isolated and cloned into vectors for expression in target cells, such as neuronal cells, in particular, certain types, populations, or classes of neuronal or interneuron cells as described.

### Example 2—the Isolated Enhancer Sequences Showed Specificity for Targeting Specific Interneuronal Cell Types

To examine the ability of the candidate, isolated enhancer element sequences to target certain neuronal or interneuronal cell populations, the isolated and cloned enhancer element sequences were individually inserted into a viral vector (rAAV)-backbone containing a minimal promoter upstream of a red fluorescent reporter transgene, e.g., d-Tomato to generate the rAAV vector called prAAV-Enhancer Sequence (ME [x])-dTomato. In some cases, from these constructs, rAAV particles were then produced with the PHPeB capsid (Chan, K. Y. et al., Nat. Neurosci., 20:1172-1179 (2017)). The ability of the enhancer sequences to restrict expression of the reporter gene (e.g., transgene) to target neurons and interneurons in brain was assessed by injecting each enhancer-containing rAAV vector systemically into animals (adult mice) and analyzing the co-localization between the expressed reporter across brain structures including the cortex. (FIG. 4). After 3 weeks, all virus vectors showed strong and sparse expression within the cortex, as well as across multiple brain regions, and drove expression of the reporter gene harbored in the virus vector exclusively in the target neuronal/interneuronal populations. As seen in FIGS. 5A-5H, cells in brain sections of mice injected with indicated enhancer-AAVs expressed the reporter gene (dTomato) (first and third columns of images). Genetically-labeled interneuron subtypes are shown in the second column of images. In the merged images (third column), dTomato-positive cells that co-localized with cell type-specific markers are shown in white.

In particular, the images shown in FIGS. 5A-5H demonstrate the results of immunohistochemical (IHC) staining analysis for detection of the dTomato reporter expressed in brain sections following systemic in vivo injection of the pAAV-ENHANCER ELEMENT-dTomato vectors harboring the sequences of the isolated enhancer elements identified and described herein. Each vector was systemically injected into an animal (mouse) allowing for detection of specific cells transduced by the vector. For example, a pAAV-ME1-dTomato vector was used to obtain the images shown in FIG. 5A; a pAAV-ME2-dTomato vector was used to obtain the images shown in FIG. 5B; a pAAV-ME3-dTomato vector was used to obtain the images shown in FIG. 5C; a pAAV-ME4-dTomato vector was used to obtain the images shown in FIG. 5D; a pAAV-ME5-dTomato vector was used to obtain the images shown in FIG. 5E; a pAAV-ME6-dTomato vector was used to obtain the images shown in FIG. 5F; a pAAV-ME7-dTomato vector was used to obtain the images shown in FIG. 5G; and a pAAV-ME8-dTomato vector was used to obtain the images shown in FIG. 5H. The results presented in FIGS. 5A-5H demonstrate that the enhancers in the expression vectors specifically targeted and restricted expression of the dTomato reporter gene in specific interneuron cell types. FIG. 5A presents images generated following injection of the pAAV-ME1-dTomato vector containing the ME1 enhancer element of SEQ ID NO: 1, which showed reporter gene expression in PV-expressing basket interneurons. FIG. 5B presents images generated following injection of the pAAV-ME2-dTomato vector containing the ME2 enhancer element of SEQ ID NO: 2, which showed reporter gene expression in PV-expressing chandelier interneurons. FIG. 5C presents images generated following injection of the pAAV-ME3-dTomato vector containing the ME3 enhancer element of SEQ ID NO: 3, which showed reporter gene expression in SST-expressing chandelier interneurons. FIG. 5D presents images generated following injection of the pAAV-ME4-dTomato vector containing the ME4 enhancer element of SEQ ID NO: 4, which showed reporter gene expression in Lamp5-expressing interneurons. FIG. 5E presents images generated following injection of the pAAV-ME5-dTomato vector containing the ME5 enhancer element of SEQ ID NO: 5, which showed reporter gene expression in Lamp5-expressing interneurons. FIG. 5F presents images generated following injection of the pAAV-ME6-dTomato vector containing the ME6 enhancer element of SEQ ID NO: 6, which showed reporter gene expression in Lamp5-expressing interneurons. FIG. 5G presents images generated following injection of the pAAV-ME7-dTomato vector containing the ME7 enhancer element of SEQ ID NO: 7, which showed reporter gene expression in Lamp5-expressing interneurons. FIG. 5H presents images generated following injection of the pAAV-ME8-dTomato vector containing the ME8 enhancer element of SEQ ID NO: 84, which showed reporter gene expression in VIP-expressing interneurons. The results shown in FIGS. 5A-5G reflect systemic injection of 2×10¹¹ viral particles per adult mouse. The results shown in FIG. 5H reflect the delivery of 5×10¹⁰ viral particles per mouse by intracerebroventricular injection in postnatal day 1 mice. The images illustrate the specificity of expression for the target populations as shown by immunohistochemistry for the viral reporter and the indicated marker gene for enhancers with specificity above 75%. All images show representative expression of the viral reporter encoded by the vector containing the enhancer sequence via staining in the somatosensory cortex S1.

The isolated enhancer elements, which serve as regulatory elements for gene expression, account for largely non-overlapping expression in populations of interneurons and neurons with distinct functions and developmental origins. The viral tools developed as described herein provide a means for elucidating and understanding neuronal subtypes and can be advantageously used to study normal cell function, as well as abnormalities in diseased cortex. The enhancer element sequences as described herein can be used to restrict, modulate, or regulate expression of a gene of interest in particular neuronal and interneuronal cell types, subtypes, and/or cell populations. In embodiments, viral vectors are molecularly created to contain the enhancer elements and genes of interest, among other functional sequences, for expression in the particular cell types.

### Example 3—Viral Targeting PV-Expressing Interneurons in Mice

The enhancer elements that target PV-expressing interneurons (e.g., mouse and human enhancers of SEQ ID NOs: 1, 9, 2, 10) provide agents for use in targeting fast-spiking neurons (e.g., basket and chandelier cells), which collectively constitute 40% of all cortical (GABA-ergic) interneurons. These neurons exert a strong level of inhibition over local networks, and their dysfunction has been directly implicated in neurological and neuropsychiatric disorders, such as neuropsychiatric disorders and the symptoms thereof, e.g., seizures, schizophrenia, or bipolar disorders. As described herein, other isolated and cloned enhancer elements are provided that specifically target other types of neurons and interneurons.

Adult mice systemically-injected with rAAV-Enhancer Element-dTomato vectors showed detectable expression of the viral reporter after one week and reached a high and stable level of expression after 3 weeks.

In particular, the ME1 enhancer element of SEQ ID NO: 1 was shown to have specificity for targeting PV-expressing basket interneurons (FIG. 5A). The ME2 enhancer element of SEQ ID NO: 2 was shown to have specificity for targeting PV-expressing chandelier interneurons (FIG. 5B). In some cases, optogenetic and chemogenetic studies may be carried out using constructs containing the isolated SST-expressing neuronal cell enhancer ME3 of SEQ ID NO: 3 or its human counterpart HuE3 of SEQ ID NO: 11. Such optogenetic studies can use the ChR2 gene in the viral construct, while chemogenetic studies can use a Gq-DREADD gene in the viral construct. The enhancer element sequences described herein may be advantageous for clinical intervention to reduce seizures and associated neuropsychiatric disorders, e.g., schizophrenia. In addition, targeting of SST interneurons in the cortex by enhancer ME3 of SEQ ID NO: 3 or its human counterpart HuE3 of SEQ ID NO: 11 and targeting PV-expressing chandelier interneurons by enhancer ME1 of SEQ ID NO: 1, ME2 of SEQ ID NO: 2 or their human counterparts, HuE1 of SEQ ID NO: 9 and HuE2 of SEQ ID NO: 10, respectively, by way of nonlimiting example, may provide beneficial uses for clinical intervention to reduce seizures and associated neuropsychiatric disorders, as well as to be therapeutically effective in cognitive disorders and cognition.

### Example 4—Materials and Methods Associated with the Above-Described Examples

Methodology overview. Briefly, to identify and isolate cell-type specific enhancers, cells isolated from the mouse cerebral cortex were subjected to the method described in Example 1 involving single cell assays and multiomics to identify candidate enhancer element sequences in regulatory regions of genes expressed in neuronal cells, or specific types of neuronal cells, e.g., interneurons. The various isolated enhancer element candidates were then individually cloned into an adeno-associated virus vector and were screened to identify those isolated enhancer sequences that were specifically expressed in certain types of neuronal cells, e.g., interneuronal cells.

rAAV cloning and viral production. All viral constructs were generated using standard cloning methods and protocols in molecular biology. The plasmid pAAV-S5E2-dTom-nlsdTom (Addgene #135630; Addgene, Watertown, MA), (Vormstein-Schneider, D. et al., December 2020, Nat. Neurosci., 23 (12): 1629-1636) was used to create a standard backbone containing the elements necessary for the production of AAVs (internal terminal repeats, minimal promoter, woodchuck posttranscriptional response element).

The enhancer sequences (necessary for restricting expression to intended neuronal cell types) were synthesized as gene fragments by GenScript (Piscataway, NJ), and the reporters and effectors were amplified by PCR. The enhancers, reporters and effectors were cloned using the NEBUILDER® HiFi DNA Assembly Kit (NEB E2621) following standard and manufacturer's procedures.

Final plasmids were assembled using the NEBUILDER® HiFi DNA Assembly Kit (NEB E2621), (New England BioLabs, Ipswich, MA), following the manufacturer's instructions and standard protocol. The rAAVs were produced using standard production methods. Polyethylenimine (PEI) was used for transfection (see, e.g., Longo, P. A. et al., 2013, Methods Enzymol., 529:227-240) and OPTIPREP™ density gradient (Sigma-Aldrich, St. Louis, MO) was used for viral particle purification and isolation. Serotype I was used to produce the AAVs for local injections in mice. Serotype PHPeB was used for systemic injections in mice. Viral titer was estimated by ddPCR with primers and probes annealing to the WPRE sequence that is common to all constructs. All batches produced were in the range of 2×10¹² to 4×10¹³ viral genomes per ml. In particular, Woodchuck Hepatitis Virus (WHP) Posttranscriptional Regulatory Element (WPRE) is a DNA sequence, which, when transcribed, creates a tertiary structure enhancing expression. WPRE, a tripartite regulatory element with gamma, alpha, and beta components, is commonly used in molecular biology to increase expression of genes delivered by viral vectors, e.g., rAAV-dTomato. (see, e.g., Choi, J.-H. et al., 2014, Mol. Brain, 7:17). All rAAV batches produced were in the range of 2×10¹² to 4×10¹³ viral genomes per ml.

Animals. Mice: Female C57BL/6J mice (Mus musculus; 10 weeks old) were obtained from Jackson Labs (Bar Harbor, ME-stock #000664). All animals were maintained in a 12 light/12 dark cycle with a maximum of five animals per cage for mice and one animal per cage for rats. All animal (mouse) maintenance and experimental procedures were performed according to the guidelines established by the Institutional Animal Care and Use Committee at the Broad Institute of MIT and Harvard.

Mouse systemic injection. For systemic injection in adult mice, approximately 10¹¹ viral particles were injected in the retro-orbital sinus per animal. Post-operative monitoring was performed for five days post injection.

Surgery. For retro-orbital vein injection, animals were anesthetized under isoflurane (1-3% in oxygen) and placed on a temperature-controlled heating pad. Intravenous (IV) injections were performed in the retro-orbital plexus. In particular, the animal (mouse) was placed in a funnel-shaped nose cone connected to a non-rebreathing apparatus (Surgivet, Dublin, OH) and the needle was inserted, bevel down, at the medial canthus, into the retroorbital sinus. Up to 150 μL of supernatant containing replication-defective rAAV vectors were injected into the tail vein or retro-orbital plexus. Following injection, the eye was held shut for a minimum of 30 seconds to ensure homeostasis.

Immunohistochemistry (IHC). Animals injected with virus vectors were euthanized with Euthasol (Virbac, USA) and transcardially perfused with 4% paraformaldehyde (PFA). The brains were placed in 4% PFA overnight, and then were sectioned at 50-60 μm (in particular, 50 μm) using a Leica VTS1000 vibrosector. Floating brain sections were permeabilized with 0.1% Triton X-100 and phosphate buffered saline (PBS) for 30 minutes, washed three times with PBS, and incubated in blocking buffer (5% normal donkey serum in PBS) for 30 minutes. The sections were then incubated overnight in blocking buffer with the indicated combinations of the following primary antibodies at 4 °C: chicken anti-GFP at 1:1,000 (Abcam USA, ab13970); rabbit anti-DsRed at 1:1000 (Clontech U.S. Pat. No. 632,496); goat anti-PV at 1:1,000 (Swant USA, PVG-213); guinea-pig anti-PV at 1:1,000 (Swant USA, GP-72); rabbit anti-SST at 1:2000 (Peninsula USA, T-4103.0050). The sections were then washed three times with PBS incubated with Alexa Fluor-conjugated secondary antibodies at 1:1000 (Invitrogen, USA), counterstained with DAPI (Sigma, USA) and mounted on glass slides using Fluoromount-G (Sigma, USA). Images of brain regions were acquired using a Zeiss LSM800 confocal microscope or a Zeiss Axioimager A1 epifluorescence microscope.

Quantification and statistics. For strength of expression, fluorescence images were taken at a standardized magnification and exposure time. The average pixel intensity of the cell bodies of each cell expressing the viral reporter was recorded and reported as an average over all cells per enhancer. For quantification of co-localization, cells expressing the indicated reporter were counted using only the corresponding color channel, and then, among these cells, the number of cells co-expressing the marker of interest was counted. A cell was considered to be positive for a given marker if the corresponding signal was above background fluorescence. The ratio of cells co-expressing both markers over the total number of cells expressing only the reporter was then calculated, reported herein as mean±standard error of the mean (s.e.m), (represented as bar plots in figures herein, for example). Quantifications were performed using a minimum of two independent biological replicates. Several sections from the same animal were used when indicated. Data collection and analysis were not performed blind to the conditions of the experiments, but experimenters from different research groups performed the quantifications. No statistical methods were used to predetermine sample sizes, but the sample sizes described were similar to those reported in previous publications.

### Other Embodiments

From the foregoing description, it will be apparent that variations and modifications may be made to the embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof, such as described in one or more sections herein.

All patents and publications mentioned in this specification are herein incorporated by reference in their entireties to the same extent as if each independent patent and publication was specifically and individually indicated to be incorporated by reference.

What is claimed is:

1. An isolated, cloned enhancer element comprising a polynucleotide sequence comprising, consisting essentially of, or having at least 75%, 85%, 90%, 95%, or 98% identity to a polynucleotide sequence set forth in any one of SEQ ID NOs: 1-16, wherein the enhancer element targets expression of a gene or polynucleotide of interest in a neuronal or interneuronal cell type, subtype, or population.

2. The enhancer element of claim 1, wherein the neuronal cell type or population comprises inhibitory GABA-ergic interneurons.

3. The enhancer element of claim 2, wherein the inhibitory GABA-ergic neurons are selected from parvalbumin (PV)-expressing basket interneurons, parvalbumin (PV)-expressing chandelier interneurons, Somatostatin (SST)-expressing interneurons, Vasoactive Intestinal Peptide (VIP)-expressing interneurons, or Lamp5-expressing interneurons.

4. The enhancer element of claim 1, wherein the enhancer element comprises
SEQ ID NO: 1 or 9 having specificity for parvalbumin (PV)-expressing basket interneurons;
SEQ ID NO: 2 or 10 having specificity for parvalbumin (PV)-expressing chandelier interneurons;
SEQ ID NO: 3 or 11 having specificity for somatostatin (SST)-expressing interneurons;
SEQ ID NO: 8 or 16 having specificity for vasoactive intestinal peptide (VIP)-expressing interneurons; and
any one of SEQ ID NOs: 4-7 or 12-15 having specificity for Lamp5-expressing interneurons.

5. An isolated, cloned enhancer element comprising
a polynucleotide sequence of SEQ ID NO: 1 or 9 having specificity for parvalbumin (PV)-expressing basket interneurons in the brain;
a polynucleotide sequence of SEQ ID NO: 2 or 10 having specificity for parvalbumin (PV)-expressing chandelier interneurons in the brain;
a polynucleotide sequence of SEQ ID NO: 3 or 11 having specificity for somatostatin (SST)-expressing interneurons in the brain;
a polynucleotide sequence of SEQ ID NO: 8 or 16 having specificity for vasoactive intestinal peptide (VIP)-expressing interneurons in the brain;
a polynucleotide sequence of any one of SEQ ID NOs: 4-7 or 12-15 having specificity for Lamp5-expressing interneurons in the brain.

6. A viral vector comprising the enhancer element of claim 1.

7. The viral vector of claim 6, further comprising a gene or polynucleotide of interest.

8. A viral vector comprising an enhancer element comprising the sequence of any one of SEQ ID NOs: 1-16.

9. A viral vector comprising
an isolated, cloned enhancer polynucleotide sequence selected from any one of SEQ ID NOs: 1-16, or a functional portion thereof, and a gene or polynucleotide of interest for expression in a GABA-ergic interneuron cell, cell type or subtype of the brain cortex;
an isolated enhancer polynucleotide sequence of SEQ ID NO: 1 or 9, or a functional portion thereof, and a gene or polynucleotide of interest to be expressed in parvalbumin (PV)-expressing basket cells of the brain cortex;
an isolated enhancer polynucleotide sequence of SEQ ID NO: 2 or 10, or a functional portion thereof, and a gene or polynucleotide of interest to be expressed in parvalbumin (PV)-expressing chandelier cells of the brain cortex;
an isolated enhancer polynucleotide sequence of SEQ ID NO: 3 or 11, or a functional portion thereof, and a gene or polynucleotide of interest to be expressed in somatostatin (SST)-expressing cells of the brain cortex;
an isolated enhancer polynucleotide sequence of any one of SEQ ID NOs: 4-7 or SEQ ID NOs: 12-15, or a functional portion thereof, and a gene or polynucleotide of interest to be expressed in Lamp5-expressing basket cells of the brain cortex;
an isolated enhancer polynucleotide sequence of SEQ ID NO: 8 or 16, or a functional portion thereof, and a gene or polynucleotide of interest to be expressed in vasoactive intestinal peptide (VIP)-expressing cells of the brain cortex.

10. A viral particle or virus-like particle comprising the viral vector of claim 9.

11. A pharmaceutical composition comprising a viral vector, viral particle, virus-like particle or cell comprising a polynucleotide of claim 1, and a pharmaceutically acceptable vehicle, carrier, or diluent.

12. A method of restoring normal levels of target gene expression in GABA-ergic neuronal cells in which expression levels of the gene are deficient or defective, the method comprising contacting the cells with an effective amount of the viral vector of claim 9 to restore normal levels of expression of the target gene in the GABA-ergic neuronal cells.

13. A method of delivering a gene or polynucleotide of interest for restricted expression in a GABA-ergic neuronal cell, the method comprising: contacting the GABA-ergic neuronal cell with a recombinant adeno-associated virus (rAAV) vector, a viral particle, a virus-like particle, or a pharmaceutical composition thereof, comprising a polynucleotide sequence of the gene or polynucleotide of interest, or a functional portion thereof, and an isolated, cloned enhancer element polynucleotide sequence selected from one or more of SEQ ID NO: 1-16 that restricts expression of the gene or polynucleotide of interest in target GABA-ergic interneuron cells of the cerebral cortex of the subject.

14. A method of treating, abating, or ameliorating a neurological, neurodevelopmental, neurodegenerative, neuropsychiatric, neurogenetic, or neuromuscular, disease, disorder, or pathology, and/or the symptoms thereof, in a subject, the method comprising administering to a subject in need thereof an effective amount of a polynucleotide of claim 1 or a viral vector, viral particle, or other delivery vehicle comprising the polynucleotide, or a pharmaceutically acceptable carrier, excipient, or vehicle thereof.

15. An isolated, cloned enhancer element comprising the polynucleotide sequence of SEQ ID NO: 3 or 11, or a viral vector comprising the enhancer element, or a functional portion thereof, wherein the enhancer element targets somatostatin (SST)-expressing interneurons expressing one or more of Satb1, Ccna1, Calb1, or Smc2.

16. An isolated, cloned enhancer element comprising the polynucleotide sequence of any one of SEQ ID NOs: 1, 2, 9, or 10, or a viral vector comprising the enhancer element, or a functional portion thereof, wherein the enhancer element targets parvalbumin (PV)-expressing interneurons expressing one or more of Lpl, Prss23, Cntnap5b, Plcxd3, or Elf5.

17. An isolated, cloned enhancer element comprising the polynucleotide sequence of SEQ ID NO: 8 or 16, or a viral vector comprising the enhancer element, or a functional portion thereof, wherein the enhancer element targets Vaso-active Intestinal Peptide (VIP) interneurons expressing one or more of Proxl, Vip, Npy5r, or Grpr.

18. A computer-implemented method for cell type-specific enhancer identification, the method comprising the following steps:
a) quantifying peaks by analyzing single cell Assay of Transposase Accessible Chromatin sequencing (scATAC-seq) data to call peaks present in cell subtypes;
b) analyzing the quantified peaks present in the scATAC-seq data of a) to identify cell type specific enhancers;
c) assigning cell type identity comprising integrating the scATAC-seq data of b) and single-cell RNA-seq (scRNA-seq) data, wherein marker expression patterns in the scRNA-seq data provide for the assignment of cell type identity for cells in the scATAC-seq data; and
d) validating cell type specific enhancers comprising identifying regions of conservation across species in cell type-specific accessibility patterns present in the cell type specific enhancers.

19. A cell type-specific enhancer sequence identified by the method of claim 18.

20. The cell type-specific enhancer sequence of claim 19, which comprises a polynucleotide sequence selected from any one of SEQ ID NOs: 1-16.


SEQUENCE LISTING (17 sequences)

SEQ ID NO: 1 - DNA, 664 bp, Mus musculus
  source 1..664: mol_type = genomic DNA
agaccgctgttgcagtatttcagggaacaagatcagaatggtatagcctacctgaaaaataggcaatcctttcaaaattaaatgcactttctcttgggtaggagacaagataatctatgataagggaagagagatgtctccagcttttattttttttcttgtaatttgcatttaagccctggtaacagtttaatatgaaggtgactatggcagctccatttattcaatctatcaatactctgttgtcacaggaggagccatgaggacttgagtgcagaggaggaatccccacaaagcagtcttcactcttatcatagcttccatgtgcccttcttattcagcagctctcattaaaggaacactatctttagcacagggattctacttgacattgacaaggacaggctcagtagcaaatgggccaaaactatgaataggcgattcacagcagagtaaaatccaatggcagcagacataaaaagaagctcaaaatgacaaacaagtcagacaaacacaaattcaagtaacaataaaaaatggagcactggtttgactcgtacacaagcaaacagacacaaacaaaacaaaagagtcattacacctggggctaaagacttctcttatgtattgcaaagtggaaataagacctccaggcaaaatgtga

SEQ ID NO: 2 - DNA, 525 bp, Mus musculus
  source 1..525: mol_type = genomic DNA
ggtgtatttcctgtgtgggctaatgtagctaactttgtttggactccaggtttctgaggcagtttcaaattcttgatttatgggttcatttttggatcatggcagcctccctgctgaaacccccatagtttcccttcaataaggaaggctggatgagatcacagctgaggaatcctggtggtgtgtactgtgttatttctctcctagtgctttgcctacacccttccatcatgcttacatttggacctgaaaataattggttgaagctgctcctcacttttagccttttatccgagtttgacagtacatcaccccaaataacacggactataaatggcttgaacctaatgagtgctctttgtggacaagccgtccattctccttttcttgagagggaaattaccggctcctaagcttctatttccagttctatagaatgtgggcgctaatttgaaagcagcctcataaaatggaagcaaacaggctaattagaaatatttatgggcctgttagtgaatatatgcc

SEQ ID NO: 3 - DNA, 645 bp, Mus musculus
  source 1..645: mol_type = genomic DNA
tttcagcataagcaagatggaatggacaatctcttatttcatcctaagttttacaggaggtccttgttttatgtaaatttccaaccatttcaaaagctctttatgtgtgtattttttttttaaccatttttgacagtttcaactctttcttctcccatgatttctttttgctctcaactgttatgggagagacaccaggttctgtatgcatttctgctttgggttgaaaattttatgcacttcaaatcaagcttcattcacatagctaaattcactgaggatacttcttccctttgaaaatttgacaagattcttaacaacccttccccgttctcctcatatttgttgagtttcctagtgcatgtccttctccctgcccccagaataactagcaggtgtgacacaggcatgcattttgcattttccaagtctgtgatgctaactttaggcctagaaacattagcagatttcctctttttatcatgcatacatttgctgcttctgttatccaatctagtcactttaaagaattttcttatttcttttgggacctgtgcaatctgttgcaagctagaaatgtccatggctagaccaataaattcactagataaatttacctgtggccacattatttaaaagtctgtg

SEQ ID NO: 4 - DNA, 629 bp, Mus musculus
  source 1..629: mol_type = genomic DNA
aaaggatcttggccctgtacgcctaagcatcccttcggtttggtagcaatggatgattgtatgcaccattctggttcacatgagttttctttctttcttttgacagcatctgattatattagtgtgtattcatcaggaacaaaatggttccatcattattcaagtgacttggattcagaatcactagacctatcgaatatgcagatgcaaaggaaagttggtctattcagcctatcccgcaacaagccaagaaaaccctggcaccaatcaggaacaacaaaaatggaagaaagtatgtgcttaataatcggtatcattctctgaagtaaataaaagtgctcagattagtggcaaagctcattggttcttttaatatcaattgtaatgccatgaatttttcccttagctactgtttcttattgaaatgaactgaaataactctaaaagaaacatctattatatgactgcaaatgttggagcagatataaatctgaataggcttatgagtagagtttctatctttgccactggcttttaattattttcctaaatcacatataactttgatgaaagtgaatattgctattttctgtcagccatggttgaatgacatcaacacaggagccatt

SEQ ID NO: 5 - DNA, 784 bp, Mus musculus
  source 1..784: mol_type = genomic DNA
gctgtaagatgcccgagaggcagaatctgaagtcatcgtctcttctctcttttttgttgttttcttgagatagagtcttgctctacagctcaggatagccttaaacctgggatatcctcctgcttcctccttctgaatgctgagatttaagggtgcatctaccacagcagtctttgtctttagaaaacttctgaacatatttgaaaagtagaaacatggaaaatactttgtatctgtgcttccaaagaaagatctatatttgtgttctgtgctctgcttgccatattggccctgtgcattgatgaacagtgaagttaccttgttaagaagcatggaagggtcagaactagccactgtccctgatggccttctgctatggtaggaagaggtttgtttccaagggccgatcttatttttatgactgaattgtgtgacagcttgaagccctaaatcaacacttctccttcattgggaatgagaaagaagccaggcagggaagagaatggcagatgaaactgggattattgtcagatggaccagccctggtaaacagtgtgtttctgacacacccagcacttcttctcagatctagctggatgccagactgtgcctgccagagacatgggctataattttgttgtttgaagcagaaaaatgtgaagatatttaaaacttagttactcattccaaaatcagatggatccccaaataactgcctaaagaacagagtgtgaagagcaacctgaaagacatgggaaagttctagccaaggtgaggtacaggt

SEQ ID NO: 6 - DNA, 776 bp, Mus musculus
  source 1..776: mol_type = genomic DNA
gttatgtttcagaggctccccaaccctgctcacctgaactcatccgttattcagtgggtcacaaactctagaacatcccagatagctcccgattcagctgctctgagtggggcctgagaatttacatgtttaacaagttcccagataaagctgataatgctgctagtttggggacatttctgtgaaaaccactggttaaagcctacccatgcattttccatgttaaaatggcccgtttgctctgccgaaggctacaggttggatgtcagagtgtgagcagagaggaaaaagaccaatttgactgcgtcttctcccaagccgcaatgtgatctgaataccttcgttcatgaaggctaaaaatgtctctttagttattttaataactatatttttgtgcctggatttcttttcatttcctaattccattgaaaatggagattaaaaaaagacactatgaaatctgttccaatgaatatttctataaagcttttgacttccaaaagcaatctgattagcatgctcttccaagccacacagtcatgtgagtgggcccgctacatttctgagcagagaaaccagacttcatccctgagttgccatcgatgtgtagcatgagttcattgtctccttttctgtgggaccagaggtagcatttcattttgtttatgtgaagtagttctttaatgaactatcagcaagaattctcaagactggccatttctgtctagctggcaaacttggttttcctaattatcaccgtttaggatgccagcatt

SEQ ID NO: 7 - DNA, 592 bp, Mus musculus
  source 1..592: mol_type = genomic DNA
ctcctctgctgggcagaaatgaggctgctacagtttgacagaagtttcccttgagttacattgtggtctgtctttattaaaagcaaaataaaagtcttatatatggggaactgccaactactccatattatgccaagttttcaatgtcttgctgttttgtgggagaagcagagaggaaggaggaagttatttgtaaattcgatacaaatagatctagccacgattatcttatcactgtcagtttaaatatagagactcttttaactaaaaatcacaattagtctattaaaaatttgaaacatttcagaaagaaccatgagcatttttgatagagacaaaccgtaagtgcaaaagaaaccaagcttcaatagctctggctctttacactgaagtcagtaactacaacagggatgccagaattttactttatgcatacatcatcatttaactcagcagctgatcatcagattcctagatggatggtggtggggcaggaaagcagtataatttatgtctgctatctgaggtaagtgggtgagagtgattctgaccagtatgaaattcataggattatgtcaaaggacttgtcatg

SEQ ID NO: 8 - DNA, 399 bp, Mus musculus
  source 1..399: mol_type = genomic DNA
tattttaaatcctcattggtcaggaatgtttacattcatagtgtgaagcagagcctgaaatgccaacttagtacttcagtgtgctgctaaattgactaagcgtgctgaacagtaatagcttggtactagaagcccaatggcagggctcagtggccaaatgtttttattattacgaagtaataaggctaataagcagtgttgtgtgatggaaggcaggcttacttctatttaaagatggaatgaacagctttgatggggacaagcaaagaacacaagaacattagctgtgttccgatattcagtgcacataggccagagcttggcaaacatttctgtaatggccagatgataaacatttgaggattggaagactatagggtctccaatgcaaccactaaa

SEQ ID NO: 9 - DNA, 648 bp, Homo sapiens
  source 1..648: mol_type = genomic DNA
aggcaagtgttactatcttccaaggactaagatcagggtgatgtgaactgaggaataggcattcctcttgaaattaaagtgtgtacattttcctaggtgctattatacaagaggatctattgagtagagaagggaaaatgccataatatttatttttgtatagtttttatttaagctctactgagaatttagtgtaaaaatgaccatggccacatcatgtggtcagtatgccaagatactggtgtcacgggattcgtgaggaattcctaatgaagagtaagaattccacaatgtagtcatcattttattgcggttcgcatgtgccctgcttattcattagctctcacaaaaaggacaatacccataatgcacaaagtctccttacaactaacaaggaacaggcccaataacaaaggtgccaaagatatgaacaggcaactcccagaagagtaaaatccaaatgctgacagacatataaaaagtagctcaaaattacaaacaagtcagagaaatgcaaattaaagtaacaatgagaaagtgtgcattggtttgacaaaaaaatatgaagagccattataccttaggcaaaagagtattcctgtgcattgcaagtagaaatgagaactgttacaggtaagatgcaggcaa

SEQ ID NO: 10 - DNA, 485 bp, Homo sapiens
  source 1..485: mol_type = genomic DNA
gtttgcttccattttatgagactgctttcaaattagcacccacattctatagaaatggaaagagaatcctggaagattgtaatttccctctcaagaaaggaagaaggacaacagatcacatgtccataaagagtacttattaggttcaagccatttatagtacatgttattcggcacaacatacttgtcaaatttggataaaaggctaaaagcgagtagcagttccaatcaattattttcaggtcctcaagcaaacatgatagaagagtgtaggcaaagcatgagaagagaaatagcatggtgcccaacaccatgatttttcaggccatgaccttggctggccttccgtattgaagaaaaaaaacaaagaagctctaccgggaccagagaatggttccagaaatcaaggattcaaaagtgcatcatagcctctaaagttaggcaaagttaacattagctatgttagaccacagagaaaatatgcc

SEQ ID NO: 11 - DNA, 556 bp, Homo sapiens
  source 1..556: mol_type = genomic DNA
aacaatgtgacaaatgggaaatgtgcctaatgaattcattggtctagctaaggaaatttccagacagaatgtagaaactgccagctgacaacaaattgaagataaatttctaacagtcttaaaagaaatgaacaaaattcttagagtgacagtttgtaagcgaataatgaaagcaacaaagaaccctaggatgaaaaagataaactctgatgaaatatctgggcttaaagatatcatcagaggacttgaaattacaaaatacatgctagtgtaaaatctttatgaaaatattacaaaattttataaagaattttgtcatatttttaaagggaaaaggcatcccatatgaagctactttaaaaatggtgatgttcagtttaaaatgcataaatgttttcaaccaaaagcagagagagatagtgaaatgaatgactcttttgcagttgattatggcagcatgaaaattatgagaggagttagagttgaaactgtcagatgtagttaaaatatgcacatacaggttacgaaattgttaaacgttttcataaaataagga

SEQ ID NO: 12 - DNA, 726 bp, Homo sapiens
  source 1..726: mol_type = genomic DNA
aatggcttctgttaagatctcacttaactgcggttgatagaaaatggcaatattcaatcaaagttatatataaattaggaaaagaattagaagccggggtagcttgacacagatataaattctcactgagcttacccaaactctcatctgctccaaggcttacccaaactctcatctgcttacccaaactctcatctttgagcttacccaaactctcatctgctccaagctgtatagaagtttctttcagagttatttcagttcatttcaatgaaaaaattatcaaatataataaaacatttttggaaagacaggagggatttgggacattacaattggtattaaaagaactaatgagctttgctactgataaagtatttcttgcattcttttttggttcaaagagtgatacccaattattaattcttaagcacataaatcttttccatttttgtttttcctgctcggtgcctgaattttcttggctactactgaaatgtgttgaataggctgattctactttttatctgcatattctatggatctagtggctctgaatccaagccactataataatgatggaaacaaaatatttctgatgaacatataaccagatattgctgacaaatagaaaaaagaacaggctgatgggggccagactgtgcatatgatctgtattcctgccaaactgcatgtatgcacagccacacaaagacaaaatccttt

SEQ ID NO: 13 - DNA, 722 bp, Homo sapiens
  source 1..722: mol_type = genomic DNA
cttgtgagttgtagatgcccttaaatgccttctctatgaaaggacacagtccatacccatcagcatttttaattaggaaaactcccagaaaattatggtatggacaaaagttgaggctttaaaaaaaaattcttggaaacacggaaaatatttcacatctgaacttcagaagaaagatctattttcatattctggttttggctcaccagactggtcctgcgctttgataaatggcaaagttacctcgttaatgagcactgaacagatccggccactgtccctgcttgggctctgtgctgggcaggaagcggttttctcccccgaggccagatctcattttcatgactgaatcttgtgagatcttgaagcactaaatcaataactcttattcagtcagccctgaaaggaaggcagttatcaaaaggaacgcaggcaaagcaggtgatagcagatgaaactcggcgattattttcaaaagaaccagcaggctctgaagtctgcggatgcttctctggcacaccccaaatcttcccctcacatccagtttggtgccaagctgtgcctgtcgtcagtatagcgtgtacaatttagttgcttgaagcagaaaaaatgtgaaagtggccaatcttatgtcttacttacctagtcaaaatgcatatggaaatgcaaagtgcttatgtctatattaaaaagaaaaaacgcatccagcacagaaagacatg

SEQ ID NO: 14 - DNA, 1775 bp, Homo sapiens
  source 1..1775: mol_type = genomic DNA
gtcttagtgaggaatgctggggcctatggttagataacatttcattcaccttgttatacttcagaagttctcatatgtgtcttaagttatttattagctcagtggttagattctagtcttgtccatgtttttgaggtggtttttttccccctggaatttggactgatttctaaattctttctgagcaacaagtacccaaactaacattttcatatttgtcttccattttttctattaagctcattaaaattactactaccttttttcctgaggccctgcaagctaaagcttattccttgcaatataggcaagaaaaatgagtcagattgccactgcattactcttctgtaactaaagatgctttaagtctaacatctggatagattttgcccaaaattaacctttgtttttattctgtttccacaaaaacgcctcttattgaaaatctgtttggctttgtatttcagacaaaagggtactggttttccagcctattcacttagatttcaaatggcattgatctcttcctataagcattgttaaactggggttaagcattttattaattatcactaggtgctatttcatttaaaaaattatttgttatattcaacaggtattcagatgggtaaagtttatatcttccaggctaccaaagttccagacaaacatgataattgactcaagggttccagcctgtgccactgcaggaaggactgagtccctataaatcattagataaggtagtgagagatttctatgcctccagatcagatagagatgacagctcctgttcaggatgaagcagttacagaagatgaaacctctgtccctctacaaccttttaggattaaggaagagtgcacagtctctgaggagagatgaaaaaggaattcagcaggacttgtttcacaagatacagaccacaaagactgctgataaaacaggatgtggtaaagaagtctgccaaaacctgccaaaaccaagatggctatgaaaacaacctctggtcattctcagtgctcattatacactaattataatatattagcataccaatggaaactccaccaacatcataacagtttacaattacaattacagttacaaatgccgtggcattgtcctgaaactaccctatatgatctaaacgggagaggatccctcaattctgggaactctcagctgctttcccagaaaactcatgaataatctactccttgtttagcacataatcaaaaaataaccataagtatagtcagtcaagcagcccatgctgctatctgcctatggggtagccacccttttattcctttagttaataaagttgctttcactttactctgtcactttactgctggctttcactttactctatcactttatactttattgctggctcttgaattctttcctgcacaagccaagaacccatgtgacctcccaagctgagttccaattttgaggtttgccctgtgacatctttggcttatgaaagcaatctgattaagatccagcttaaaattataatcatgtaagtcaactctactacatagttttgagcacagaaatcagaattcatgctatgaattgccctaggcttgtagtctgaacttgttctctccctatttgcaggggtcagtggtaggatttaatctgtttatacggattaactgctgtatagattacctacagcaattcttaagacaggctgtttcctttcatttagtaaaactttgttttcttaatttgtcaatgcttagtgtgccaacaat

SEQ ID NO: 15 - DNA, 577 bp, Homo sapiens
  source 1..577: mol_type = genomic DNA
ctcctactgggaggaaatgaggctgctattggtctgactgaagttccccttaggctatatgtggtctgtctttattaaaagcaaaataaaggcctcacatggggacttggcaactattacaagggttgtgctgggtttttcgattcttgcttttttgttgggggtgtatgaaaggaatcaacagatccatccaagactattttatcattgtcaacttacatgcagaccaccctgacacccccactcactattagtctgttgaaaatgggaagaatttgagagggaaacactaacatttttaatagaaacaattcttatagccaaataagaactaagctacaaaggttctaattatacattaggtattcaataaacataatggtgattccagaattgtattttgtatgccatcatctaatttataagttggcatcatcagattcaaacactgagtggaaaaaaactcagcaatataaattatataagcaatcatcacgtgagtgggtggaaatgattctgaaacagataattctgaaatcaatcctgaaataggactatttgaaaggattaacttctg

SEQ ID NO: 16 - DNA, 354 bp, Homo sapiens
  source 1..354: mol_type = genomic DNA
gattttaaaccctcaatggccgggaactattatatccatggtgccaagcagagcctgagatgccaaattagtacttaaatatgctgttagactaagcatcctgagcgatgagtgcttagtactggaagaccaacgacaggggtcagtggcctattgtcttcattatcacaaattaataagtctaacagacggtgctgtgtgagatggaaaatgagcttacctctgtttaaagaaggaaccaacagctttggtgtggacaggcagagaacaacagaaacggtaaaacttttggcaaacattttctgcagcatattaggctttgtggaccatacagtctctattgcaactgctcaa

SEQ ID NO: 17 - protein, 590 aa, synthetic construct
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL